(19) United States
(12) Patent Application Publication
(10) Pub. No.: US 2025/0046436 A1
(43) Pub. Date: Feb. 6, 2025

(54) HUMAN RESOURCE MANAGEMENT AI-OPTIMIZATION SYSTEMS AND METHODS

(71) Applicant: RRSP INDUSTRIES, BROOKLYN, NY (US)

(72) Inventors: Aaron S. ZAK, Forest Hills, NY (US); Alexander Christian GRZESKA, Remagen (DE); Hakan YOZTYURK, Arnstadt (DE); Edward Ronald GRIFFOR, Gross Pointe Park, MI (US)

(21) Appl. No.: 18/717,342

(22) PCT Filed: Dec. 8, 2022
(86) PCT No.: PCT/US22/52212

§ 371 Date: Jun. 6, 2024

Related U.S. Application Data
(60) Provisional application No. 63/287,601, filed on Dec. 9, 2021.
(60) Provisional application No. 63/288,935, filed on Dec. 13, 2021.
(60) Provisional application No. 63/430,782, filed on Dec. 7, 2022.

(51) Int. Cl. G16H 40/20; G06N 20/20

(52) U.S. Cl. CPC G16H 40/20; G06N 20/20

(57) ABSTRACT

A method to optimize human resource management within emergency medical services (EMS) according to one approach may have the steps of: receiving inputs from at least one or more the data sources such as traffic conditions, weather, incident location of emergency or non emergency call, call type, dispatch type, latitude and longitude of incident location, age, sex, chief complaint, incident date and time, holiday, day of the week, call classification, emergency department population status, incoming EMS service requests, and the like and combinations thereof; providing a scheduling module for building one or more predictive assessment values; the scheduling module outputting automatically a suggested scheduling template; employing one or more machine learning models to generate the one or more predictive assessment values that relate to the comparison of the inputs of the evolution of data over a time interval using machine learning; employing a global positioning systems (GPS) device to provide geo-location information of EMS equipment.

### CROSS-REFERENCE TO RELATED APPLICATIONS

This application is a PCT application and claims priority from U.S. provisional application 63/287,601 filed Dec. 9, 2021; U.S. provisional application 63/288,935 filed Dec. 13, 2021; and U.S. provisional application 63/430,782 filed Dec. 7, 2022, which are hereby incorporated herein by reference in their entirety for all purposes.

### FIELD

The present embodiments relate generally to information and communication technology adapted for the health care industry related to emergency medical services (EMS), and specifically to information and communication systems and methods adapted for the health care industry related to EMS using machine learning.

### BACKGROUND

The field of healthcare currently faces numerous challenges in the management of its resources such as staffing, supplies, breakdowns in communication and the like. These challenges are particularly acute when large-scale mass casualty incidents occur. Rising health care costs and staff shortages have also contributed to these challenges.

Attempts are known in the art to address these challenges using machine learning. For example, systems sold under the tradename Avantas (for scheduling) and Hospital IQ (which combines machine learning based-AI, communications, and automated workflows to optimize work environments). Machine learning is also known for ‘cleaning’ diagnostic imaging, and assistance in differential diagnoses.

Other applications using artificial intelligence in the health care arts may include natural language processing (NLP). NLP gives computers the ability to understand text and spoken words in the way humans can. In other words, NLP can be used to interpret computer code, written text and spoken speech and then process the information, make comparisons and analytics, or finish written code. NLP may be applied in many applications outside of health care in the development through open AI for example, providing the ability to complete websites from basic instruction. In the health care arts, NLP can be used in cross analytics of lab diagnostics, guided surgeries and to extract clinical concepts from a patient's medical records, discharge summaries, lab reports and the like. One system sold under the tradename FORESEEMED uses machine learning and NLP to improve documentation to increase reimbursement rates for healthcare facilities. Another system sold under the tradename IQVIA focuses NLP to compare and analyze documents to ensure medical institutions are compliant with current regulatory standards. Another system sold by 3M under the tradename CODERYTE CODEASSIST can recognize statements about diseases and treatments within a physician's report. The software then labels the report with International Classification of Diseases (ICD) and Current Procedural Terminology (CPT) codes so that expenses can be automatically reimbursed by a patient's insurer.

Despite these advances in the art, there is a desire and need to expand machine learning into other areas of health care such as emergency medical services.

### SUMMARY

Methods and Systems are provided for the health care field related to the emergency medical services to be fully self-sustaining and operate at maximal efficiency, with a minimal number of resources, within a closed system, with the added ability to operate separately from external supply chains and resources. AI-assisted resource management methods and systems is the central infrastructure of a self-sustainable smart hospital and health care system, which can operate separated from outside resources and supply chains. The system and embodiment of systems is a supportive tool for health care professionals, facilities and EMS organizations to allocate resources appropriately, meeting demand with the resources only available to them, determined by ML machining learning predictions, models and forecasts. An extension and rearrangement of data supports many use-cases, such as but not limited to, epidemiological forecasting, supply and waste management, general research, and human resource management, with in and out of the hospital setting.

The present embodiments provide AI-assisted resource management methods and systems. The emergency medical arts lack the capability to accurately and efficiently distribute its resources to areas of most need. The present embodiments enable Emergency Medical Service agencies to adjust staffing needs to provide optimal coverage amounts without increasing the burden on staff and payroll. The present embodiments automate scheduling suggestions based on predicted future call volume, incident location, and call type utilizing machine learning. As a result, EMS agencies can more effectively meet optimal ambulance call ratios, provide adequate reimbursement and revenue, increase profits, and decrease 911 call response times to help save lives.

A method to optimize human resource management within emergency medical services (EMS) according to one approach may have the steps of: receiving inputs from at least one or more the data sources selected from the list comprising traffic conditions, weather, incident location of emergency or non-emergency call, call type, dispatch type, latitude and longitude of incident location, age, sex, chief complaint, incident date and time, holiday, day of the week, call classification, emergency department population status, incoming EMS service requests, available medical consumables, available medical non consumables, available staff, Cellular triangulation of staff, Cellular triangulation of ambulance or other mobile EMS equipment, Cellular triangulation of service base sites, GPS location of staff, GPS location of the service base sites, GPS location of ambulance or other mobile EMS equipment, identified location of needed services, dispatch requests, time of dispatch requests, latitude and longitude of dispatch requests, hospital census counts, duration of patient admittance in hospital, admitting diagnosis in hospital, discharge diagnosis in hospital, unit transition patterns of patients, unit transition date and time, unit admitting date and time, admitting unit in hospital, on-site manufacturing capabilities; providing a scheduling module for building one or more predictive assessment values; the scheduling module outputting automatically a suggested scheduling template which matches EMS call volume appropriately to provide the maximum ambulance per call ratio per shift and is configured to ensure maximum revenue for an EMS agency, and the like and combinations thereof; and employing one or more machine learning models to generate the one or more predictive assessment values that relate to the comparison of the inputs of the evolution of data over a time interval using machine learning; employing a global positioning systems (GPS) device to provide geo-location information of EMS equipment and personal communication devices in order to assess availability; and displaying to a user the results of the comparison, including the one or more comparison outcomes, and one or more predicted assessment values that relate to the comparison of inputs or to the predicted evolution of data over a time interval using machine learning.

According to one approach, the scheduling module uses incident time, location, and type to predict and forecast future call volume type and location in real-time. In another approach the scheduling model outputs one or more of automatic scheduling, tracking of epidemiological data for research, and resource/supply management. In another approach the scheduling model outputs predicted medical consumable needs by agency. In another approach the machine learning machine learning models utilize Ensemble learning+neural networks, Decision tree and deep reinforcement learning via call simulations to create Model-free algorithms (Soft Actor-Critic, TD3, PPO). The system of present embodiments relates to EMS and Healthcare Systems and provides a method to improve quality of care, resource provisioning and preparedness to variation in patient demand. The term patient trajectory refers to the assembling, scheduling, monitoring, and coordinating of all steps necessary to complete the work of patient care. The system of the present embodiments, starting with the emergency medical services, allows the healthcare system to operate as a federated entity and accomplish three main tasks to improve quality patient care: i) allocate resources at the patient side to meet patient demand, ii) optimize the patient trajectory relative to quality of patient care, and duration of patient care incl. time and steps involving institutionalization iii) provide decision making support for care providers to improve quality of care for the patient.

The system of the present embodiments provides continuous analysis of three profiles to provide the optimal patient trajectory 3100 and quality of care throughout the care track. (see FIG. 56 and FIG. 64) The three profiles are the EMS Unit Profile 5603 on FIG. 61, the Patient Condition Profile (PCP) 5601 on FIG. 58, and the Receiving Entities Profile 6300 on FIG. 63. The system analyzes the EMS Unit Profile 5603 on FIG. 61, ensuring the adequate level of care is available near the location of the emergency call. The EMS Unit Profiles may include Advanced Life Support (ALS) or Basic Life Support (BLS) unit types. The system learns and analyzes the PCP 5601 on, for example, FIG. 58, to determine the patient trajectory based on patient outcome and time to treatment. The system can analyze the receiving entities profile 6300 on FIG. 63 for characteristics such as bed availability, staff availability and supply availability, along with predicted patient load by patient condition for that facility. These profiles are encoded to enable storage and processing using a digital computing device. The system analyzes these profiles to determine the optimal outcome and coordinates EMS unit response and patient trajectory 3100 based on current and forecasted conditions. The analysis of these three profiles together results in improved distribution of resources, decreased burden on the healthcare system, and improved quality of care.

One method of the system has two components: i) the Patient condition Profile (PCP) engine 5801 and ii) the PCP Database 5802, which receives the information gathered from a 911-caller by the dispatcher and generates, stores and updates the Patient Condition Profile. The Patient Condition Profile is generated 5806 at the time of the dispatch center receives the 911 call from the 911 caller 5701. The data of the Patient Condition Profile is derived from the caller's answers to the 911 call taker's questions according to the conventional scripts. The Patient Condition Profile then assists the system in determining the response based on characteristics such as optimal outcome. This method performs continuous intelligent analytics to adapt to changes in the EMS unit 5603 and receiving entity profile 6300 with relation to the changing patient condition throughout the patient trajectory 3100. Some inputs to this method may include patient assessment findings, complaints, point of care diagnostics, and the like.

One method of the system (Intelligent Dispatch System) (see 5600 on FIG. 56 and FIG. 57) performs analytics of Patient Condition Profile 5601, Triage 5602 (incl. patient criticality assessment), and Response 5603 to coordinate the outcome of the response. The patient care needs are based on the Patient Condition Profile and requirements of quality care, optimal outcome and supply and care provisioning around the changing patient condition. Triage 5602 inputs include Patient Condition Profile, to determine the optimal and appropriate EMS unit to respond and is performed by analyzing all the factors already discussed to determine optimal outcome, and provisioning of resources. The system applies its analysis of these components for the EMS response to design and coordinate EMS and Hospital daily operations and to prepare and optimize patient care treatment and outcome.

One method of the system (see 5603 on FIG. 61) continuously analyzes and updates the Patient Condition Profile to provide intelligent EMS response to the patient's condition. The system adapts to the changing patient condition. Optimal choice of EMS unit response is determined by analyzing the patient condition 5601, EMS unit 5603, and receiving entity profiles 6300 for the most optimal patient outcome. This information sharing method of the system improves preparedness for changes in patient condition throughout the entire time the patient is in EMS care. In one implementation the system provides treatment and care coordination, for example air evacuations, cardiac arrests, hospital diversions and the like. The exchange of information occurs throughout the entire care of the patient in the EMS unit, until the patient is handed off to a higher level of care at the receiving entity.

One method of the system 4800 aligns healthcare resources, incl. supply and personnel distribution, to better meet patient demand. The information provided by the system aligns acquisition and provisioning to ensure the supplies needed to respond to the patient condition are present on the responding EMS unit. This method enables coordination from the highest level including the manufacturer to the supply base, ensuring proper supply distribution of supplies are on the responding unit for the call. Supplies encompass all materials needed for treating the patient. This can include but is not limited to medications, equipment, and supplies, including consumable and non-consumable. The system's predictive capability improves the provisioning of healthcare professionals for EMS response, such as EMTs and paramedics, by aligning availability with predicted characteristics of future calls.

One method of the system provides coordinated and Intelligent Predictive Analytics 4200 with visual outputs from those analytics (a Visualization Function Map) (see 4301 on FIGS. 43 and 44). This method displays forecasting of events for patient demand and supports quantitative reasoning. These events can be filtered, by date, time, type of call, disease, diagnosis, and the like. Analytics provides localization based on the Patient Condition Profile 5601 for a coordinated effort to plan and prepare for all care providers along the patient trajectory. One Example includes support for scheduling, which helps coordinate and automate scheduling changes based on predicted patient demand. Another example uses the analytics' visualization to perform epidemiological studies, by providing the ability to study the differences in Patient Condition Profile within regions of the nation, state, population, specific demographics and the like.

Applications of the method of the present embodiments (which arise from the use of the overarching data set) include Emergency Medical Preparedness service that coordinates, from the Federal Level to Local Level (see 6700 on FIG. 67). Applications may also include the ability to simulate emergency responses to support decision making at all levels, including municipalities (see 4100 on FIG. 41). Imitating real-life operations to specific events with reference to forecasted daily operations, and resource availability such as supply provisioning and care provisioning, and the like, is another potential application (see 4100 on FIG. 41). The continuous monitoring of the patient condition through the trajectory 3100, and the coordination of IoT with medical devices, allows one to determine effectiveness of medical devices against patient outcome and cost, hence applications to treatment effectivity or validation studies (see 7100 on FIG. 71). Decreasing the patient trajectory decreases the cost of care, by decreasing the amount of insurance claims against a patient case, and potentially the computation of premiums, while allowing for dedicated patient care supervision. This provides the ability to analyze the effectiveness of operations, medical devices, pharmaceuticals, for patient outcome and cost benefit as well as tools for Quality Assurance/Quality Improvement (see 7100 on FIG. 71). Another application is Market Surveillance that provides the ability to simulate real-life outcomes of cost benefit, patient outcome, and burden on the healthcare system against the forecasted Patient Profiles, side effects and the potential Patient Profile and their trajectories (see 7100 on FIG. 71). The interoperability with other intelligent cyber physical systems offers opportunity to automate and coordinate provisioning of resources, supplies, traffic patterns, warehouse organizations and the like, from the highest to the lowest level (see 6800 on FIG. 68).

The system results in improved performance as measured by a performance index for EMS created and updated continuously by the system.

The system handles patient sensitive data storage and intersystem sharing with care in accordance with the Health Insurance Portability and Accountability Act of 1996 (HIPAA) and the General Data Protection Regulation of the European Union.

According to one approach, the system machine learning models learns and analyzes a patient condition profile to determine a patient trajectory based on patient outcome and time to treatment.

According to one approach, the system may analyze a receiving entities profile for characteristics including at least one of bed availability, staff availability, supply availability and predicted patient load by patient condition for that facility; wherein the profiles are encoded to enable storage and processing using a digital computing device; wherein the system analyzes the encoded profiles to determine optimal outcome and coordinates EMS unit response and patient trajectory based on current and forecasted conditions.

According to one approach, the system may have components for a Patient Condition Profile engine and a Patient Condition Profile Database, which receives information gathered from an emergency dispatch caller and generates, stores and updates the Patient Condition Profile in real time; wherein data of the Patient Condition Profile is derived from a caller's answers to a 911 call taker's questions according to predetermined scripts; wherein the Patient Condition Profile output assists the system in determining a response based on predetermined characteristics, including optimal outcome.

According to one approach, the system may have an EMS Response Module that continuously analyzes and updates a Patient Condition Profile to provide intelligent EMS response to the patient's condition; wherein the system adapts to input of a changing patient condition; wherein an optimal choice of EMS unit response is determined by analyzing the patient, EMS unit, and receiving entity profiles for the most optimal patient outcome.

According to one approach, the system may have an EMS Unit Provisioning Module that aligns healthcare resources, including supply and personnel distribution; wherein the system aligns acquisition and provisioning to ensure the supplies needed to respond to the patient condition are present on the responding EMS unit; and wherein supplies may comprise at least one of medications, equipment, and consumable and non-consumable supplies.

According to one approach, the system may have coordinated and Intelligent Predictive Analytics with visual outputs from analytics of a Visualization Function Map which includes the step of displaying forecasting of events for patient demand and supports quantitative reasoning; wherein the events can be filtered by at least one of date, time, type of call, disease, and diagnosis.

According to one approach, the system may have the step of simulating emergency responses to support decision making at all levels, including municipalities.

### BRIEF DESCRIPTION OF THE DRAWINGS

The present embodiments, as well as a non-limiting exemplary mode of use, further objectives and advantages thereof, will best be understood by reference to the following detailed description of an illustrative embodiment when read in conjunction with the accompanying drawings, wherein:

FIG. 1 illustrates an exemplary visual representation 100 of future single call type, time and location.

FIG. 2 illustrates an exemplary visual representation 202 of future multiple call types, time and location.

FIG. 3 illustrates an exemplary visual representation 300 of tracking past, present and future patient admittance in the hospital in all departments.

FIG. 4 illustrates an exemplary visual representation 400 of comparing patient admittance in two departments.

FIG. 5 illustrates an exemplary visual representation 500 of comparing patient admittance in multiple departments.

FIG. 6 illustrates an exemplary visual representation 600 how the system automatically sorts the busiest and the slowest departments in real-time and future time.

FIG. 7 illustrates an exemplary system architecture and data stream 700.

FIG. 8 illustrates an exemplary non-relational database 800 of the present embodiments.

FIG. 9 illustrates an exemplary diagram 900 for the end user.

FIG. 10 illustrates an exemplary Core module architecture and data stream 1000 of the present embodiments.

FIG. 11 illustrates an exemplary Core module—Call-prediction 1100 of the present embodiments for location and time.

FIG. 12 illustrates an exemplary Core module—Call-prediction 1200 of the present embodiments for number of call predictions.

FIG. 13 illustrates an exemplary Scheduling module 1300 of the present embodiments.

FIG. 14 illustrates an exemplary Resource & Supply Management Module 1400 of the present embodiments.

FIG. 15 illustrates an exemplary Epidemiology module 1500 of the present embodiments.

FIG. 16 illustrates an exemplary format 1600 to store and present non-sensitive medical information from the emergency medical services.

FIG. 17 illustrates an exemplary format 1700 to store and present non-sensitive medical information from the hospital.

FIG. 18 illustrates an exemplary model 1800 of how the system decreases idle time and increases revenue.

FIG. 19 illustrates an exemplary model 1900 of how the system decreases idle time.

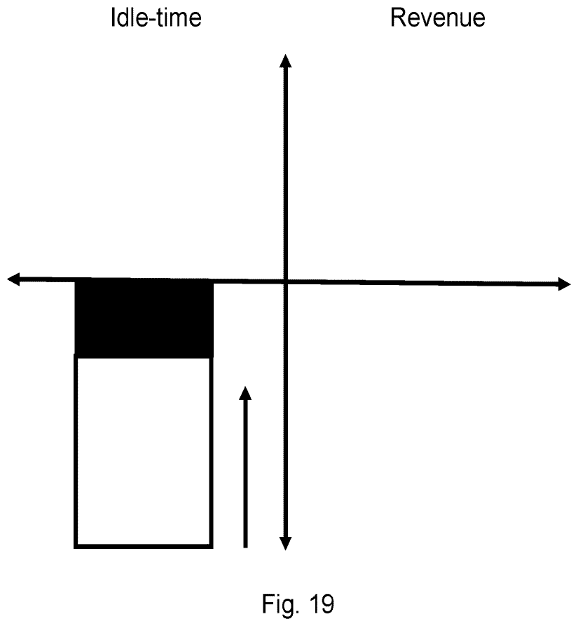

FIG. 20 illustrates an exemplary graph 2000, of the linear loss in USD of the no. of ambulances for idle-time in one hour.

FIG. 21 illustrates an exemplary table of the number of ambulances on-duty per hour, and potential loss per hour and year, with the corresponding theoretical maximum ambulances needed per hour for zero loss.

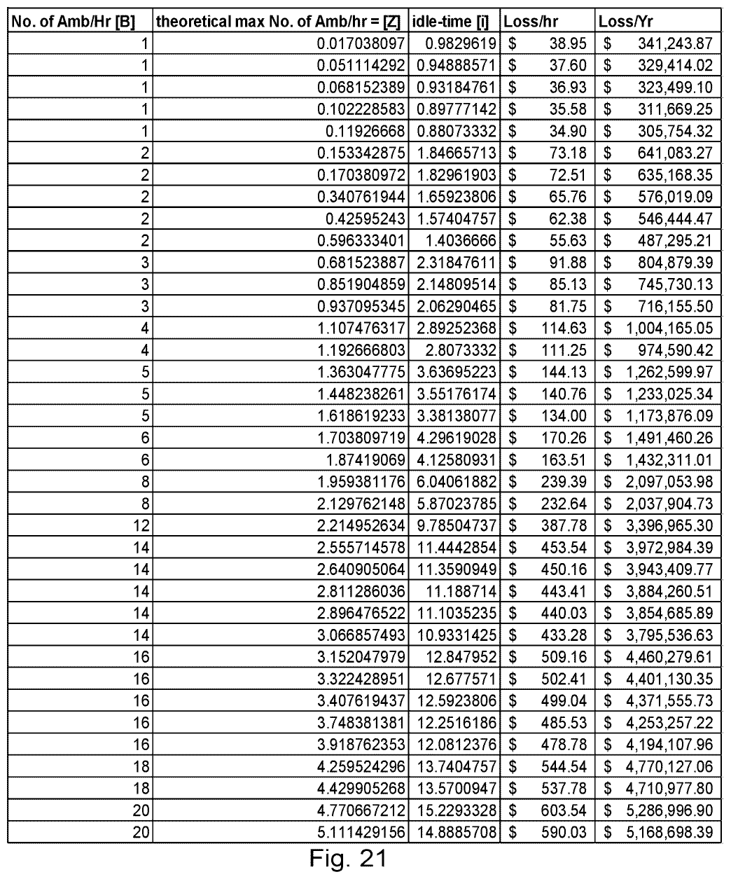

FIG. 22 illustrates an exemplary model 2200 of how the system increases revenue.

FIG. 23 illustrates an exemplary breakdown 2300 of parts associated with a virtual electrocardiograph.

FIG. 24 illustrates an exemplary breakdown of parts 2400 associated with a prehospital vascular (and tissue) imaging device.

FIG. 25 is an exemplary general system for use in implementing methods, techniques, devices, apparatuses, systems, servers, sources, and the like, in accordance with some of the embodiments.

FIG. 26 illustrates an exemplary diagram 2600 of the primitive neural networks.

FIG. 27. illustrates an exemplary diagram 2700 of the deep neural network.

FIG. 28 is a high-level overview of the system.

FIG. 29 is an overview of any Module ‘n’ of the system.

FIG. 30 is an overview of the information flows between the IoT Platform(s) and the service platform(s) of the system.

FIG. 31 is a general overview of the patients' trajectory though the healthcare field, or in other words an overview of one possible way how the patient moves through the healthcare field.

FIG. 32 is an overview of how the patient condition profile (PCP) is created.

FIG. 33 shows how the patient condition profile is plotted against the field and destination key performance indicators (KPIs).

FIG. 34 shows the patient trajectory of a patient who meets STEMI criteria or a suspected non-ST elevation myocardial infarction (NSTEMI).

FIG. 35 illustrates the system obtaining a new key performance indicator (KPI) using values between +1 and −1.

FIG. 36 shows the system adding two values to determine a patient condition value for each stage of the patient trajectory.

FIG. 37 illustrates the calculated average of all patient condition profile values at the end of the patient trajectory.

FIG. 38 shows an optimal patient trajectory with its corresponding field and destinations key performance indicators.

FIG. 39 is an illustration of the backend architecture of the system.

FIG. 40 is an overview of Module 1 (Simulation Environment) and the data it uses.

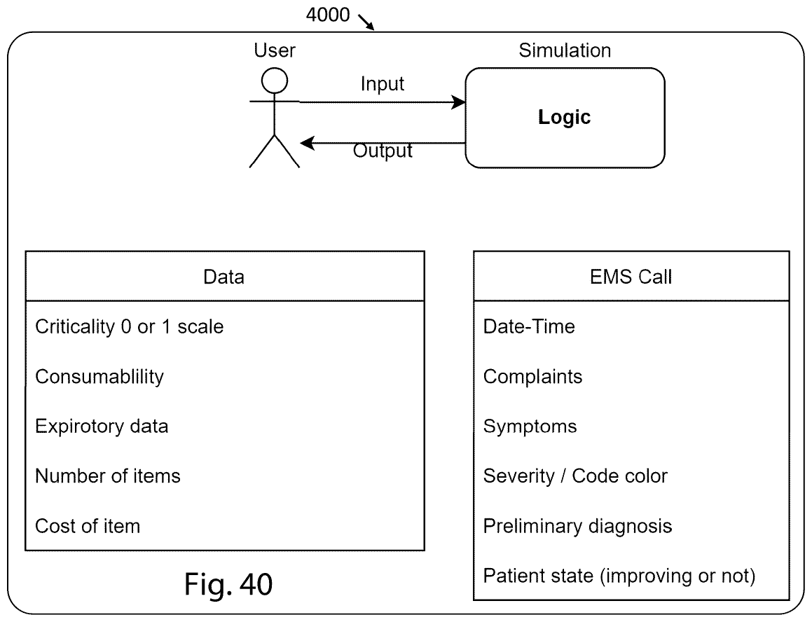

FIG. 41 shows customers and end points for Module 1 (Simulation Environment).

FIG. 42 demonstrates how a response request is processed, and new response records are added to the EMS response database.

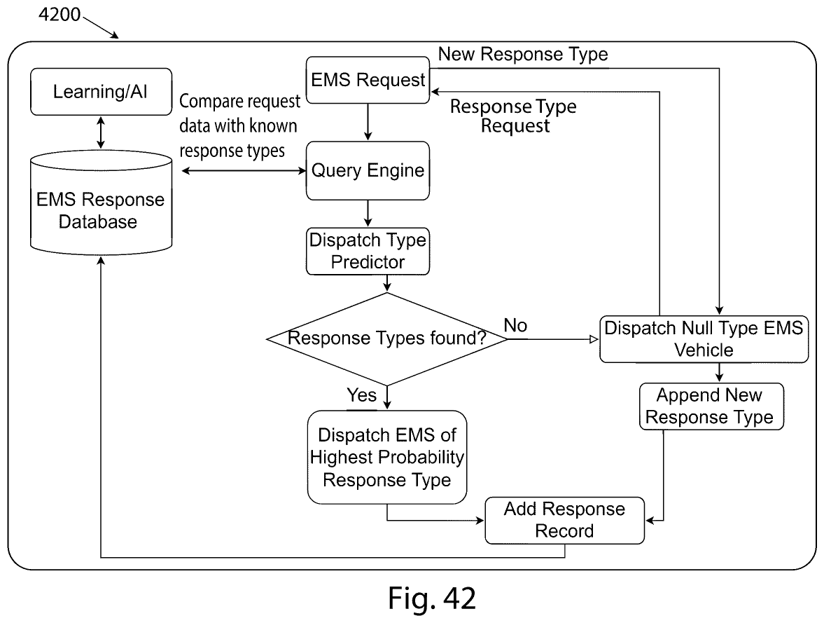

FIG. 43 shows how new EMS response profiles are added to the EMS response database and how the results are visualized via a Heat Map.

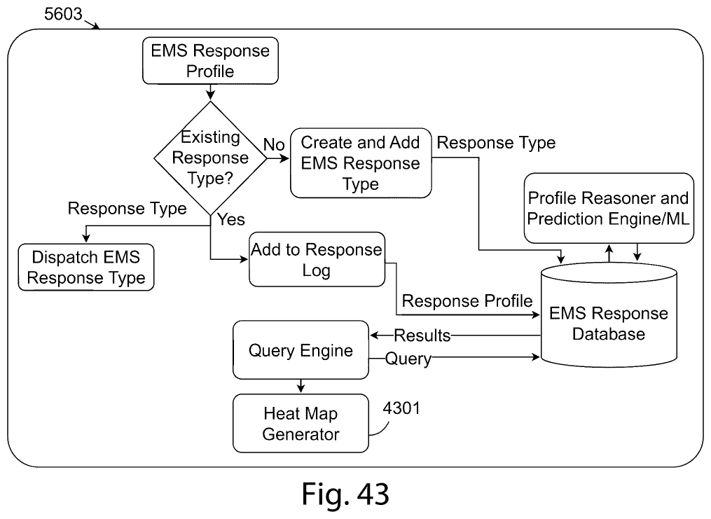

FIG. 44 illustrates how EMS trip requests (input) are processed and how reports are generated (output).

FIG. 45 illustrates the component of the EMS management services that are built on top of the systems platform and its related data sources.

FIG. 46 illustrates how patient data analytics aids in the management of resource planning in Emergency rooms (ER).

FIG. 47 show how an EMS dispatch request is processed in accordance with the EMS Unit profile.

FIG. 48 illustrates how patient data analytics aids in the management of resource planning in the emergency medical services (EMS).

FIG. 49 illustrates how EMS information is managed and how new trip records and designation in trip cases are added.

FIG. 50 shows how the system aids in the planning of EMS vehicle development.

FIG. 51 shows the problems that lead to delay in patient care and how they are related.

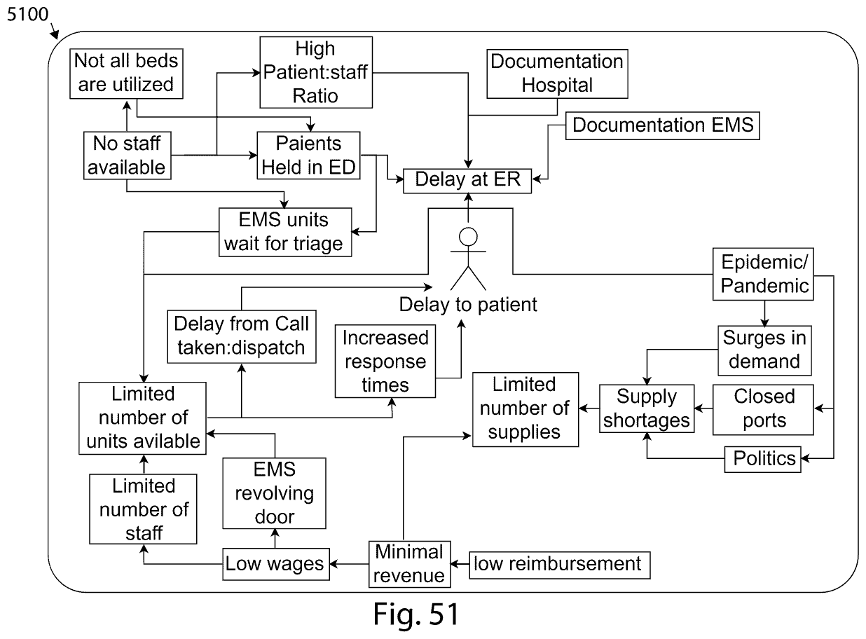

FIG. 52 illustrates the problems in the Emergency medical services and hospitals and how they lead to overcrowding in the Emergency department (ED).

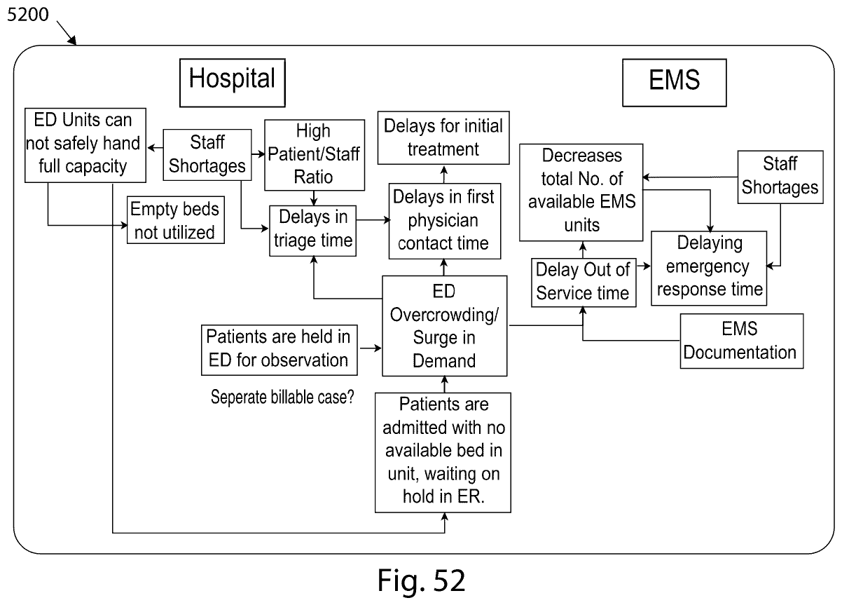

FIG. 53 illustrates a telemedicine platforms' decision-making tree for doctors in the emergency department in a 911 scenario.

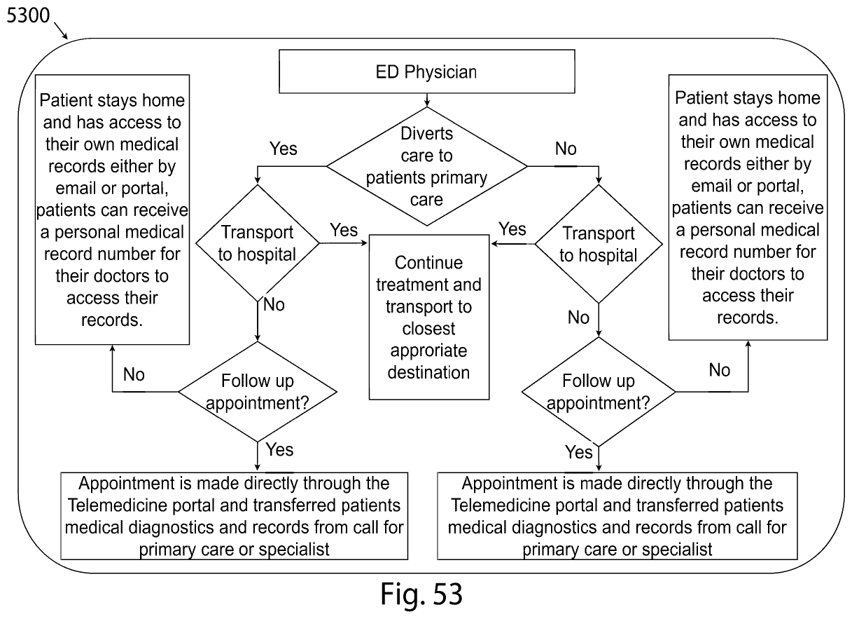

FIG. 54 illustrates a telemedicine platforms' decision-making tree for primary care physicians in a 911 scenario.

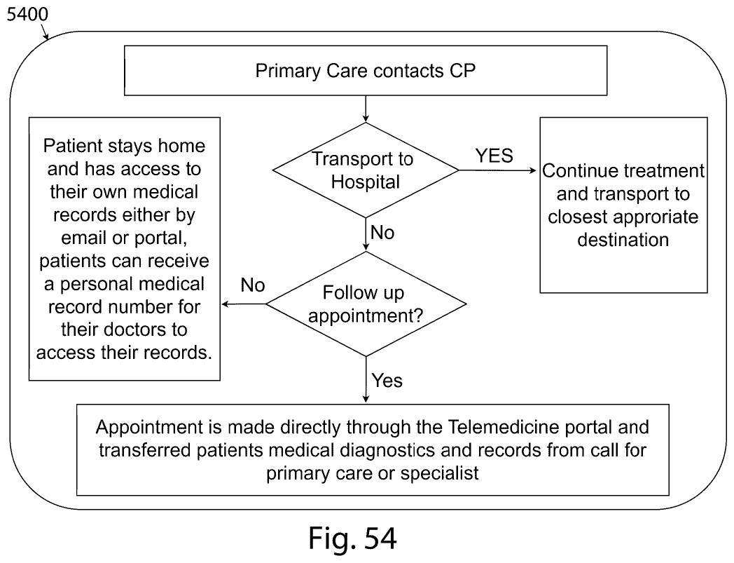

FIG. 55 is an illustration of the changing patient state space throughout the patient's trajectory in the healthcare field.

FIG. 56 is a functional block diagram of an intelligent dispatch system and relation between its different components.

FIG. 57 is a flow diagram of the intelligent dispatch system and its multiple analyses to provide proper EMS unit provisioning from the generation of the patient condition profile.

FIG. 58 is a general overview of how patient condition profiles are created, updated and stored.

FIG. 59 shows how a location specific patient condition profile is searched in the database and displayed.

FIG. 60 is a general overview of how the system analyzes and determines the appropriate triage level.

FIG. 61 is an overview EMS response analysis which involves the interpretation of the patient condition profile analytics method and ongoing triage analytics method.

FIG. 62 illustrates the workflow of an EMS unit at the site of incidence and how the patient condition profile is updated.

FIG. 63 illustrates the workflow at the destination site of the EMS unit (e.g. a hospital) and how the patient condition profile is updated.

FIG. 64 illustrates a functional block diagram of the relationship between the intelligent trajectory system with the provisioning of supplies and resources to the patient state space.

FIG. 65 illustrates the flow of information in the intelligent trajectory system between three analytics of the patient condition profile, triage and trajectory.

FIG. 66 illustrates the flow of information for trajectory analytics.

FIG. 67 illustrates the intelligent exchange of information between all stakeholders from the IoT devices and field sensors.

FIG. 68 illustrates the flow of information for supply and material provisioning based on ML predictions of the system to each respective stakeholder.

FIG. 69 illustrates the flow of information between the system and on-site manufacturing production, for supply and material automated and regulated production based on the systems ML forecasting of patient demand.

FIG. 70 illustrates the flow of information for the system to forecast optimal EMS and hospital unit distribution.

FIG. 71 illustrates the flow of information for how the system learns cost efficiency and patient outcome based on treatment plans, and sensors in the field from medical devices and the like.

### DETAILED DESCRIPTION

Inappropriate staff utilization causes increased stress on health care systems as hospitals and emergency departments often face difficulties meeting effective patient-to-staff ratios, which decreases the quality patient care and satisfaction.¹ According to a Winter et al. one of the most substantial causes of low patient satisfaction is a result of a high patient-to-staff ratio.¹ Their results indicate that fighting staff shortages is not only important to secure the quality of care but also the satisfaction of patients, which is increasingly important in the context of attracting enough patients for a sustainable and viable service¹. Moreover, volume and staffing levels indicate an agency's ability to relieve an emergency¹. Since most agencies suffer shortages, a huge strain is placed on the available staff who need to consistently and reliably fulfill demands for emergency medical services (EMS). A survey conducted by the New York State Department of Health Bureau of Emergency Medical Services and Trauma Systems survey showed that 40% of the EMS responder workforce is considered distressed². Additional recruitment of certified EMS responders however, is not considered a viable possibility by most agencies. Private EMS on the other hand seems to solve unprofitability through transports, which are guaranteed to be reimbursed by insurance. However, there is still a high bankruptcy rate among ambulance service providers³.

The present embodiments relate to systems and methods to enable improved information and communication for the health care industry related to EMS using machine learning. The present embodiments provide many new advantages for the health care industry to forecast models, make predictions of future events, to identify trends and to generate advanced analyses and recommendations within, for example, the EMS setting.

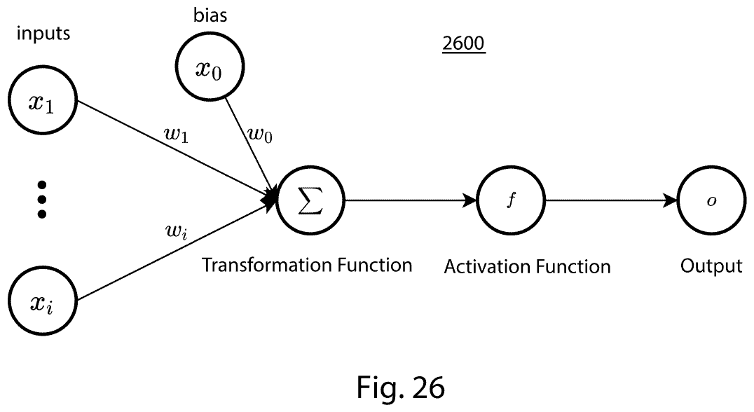

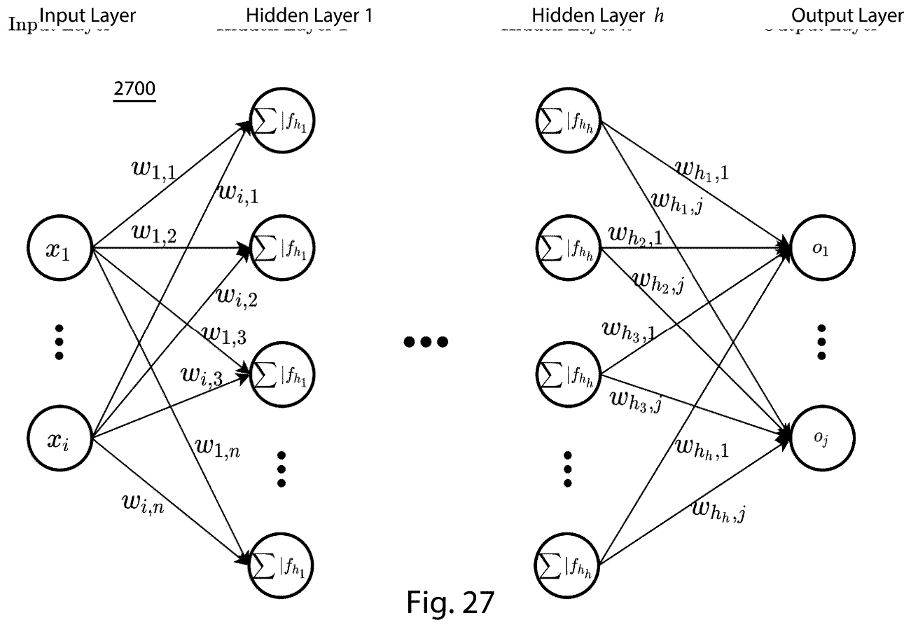

As used herein, the terms “machine learning” or “ML”, “machine learning module” or “ML module” refer to machine learning models or modules that may be arranged for scoring or evaluating model objects (e.g., documents) (see e.g., FIGS. 26 and 27). The particular type of ML model and the questions it is designed to answer may depend on the application the ML model targets including a user providing or inputting further information. ML models may include models arranged to use different training techniques or statistical techniques, including, linear regression, lasso regression, ridge regression, decision tree, random forest, logistic regression, or the like, or combination thereof. Further, in some embodiments, various heuristic methods or processes may be associated with a ML model including a user providing or inputting further information. In some embodiments, configuration information may be employed to configure or select one or more ML model for a particular embodiment or application, or the like, or combination thereof.

The present embodiments have the potential to decrease the burden of commonly high patient-to-staff ratios and other collateral problems which healthcare systems face from patient overflow. The present embodiments allow EMS and hospitals to collaborate, plan, and prepare accordingly based on future epidemiological predictions by providing a platform for doctors, scientists, and researchers to pull and automatically generate customizable reports, reducing the need for lengthy and expensive epidemiological surveys and systematic reviews. The present embodiments' machine learning algorithms, (see e.g., FIGS. 26 and 27) have the ability to re-train themselves with the real-time stream of data entering into the system on a daily basis, to provide more accurate predictions. The more users and time of use elapsed of the present embodiments, the more accurate its predictions become. In other words, over time the present systems just become increasingly more optimized in its predictions translating to increase effectiveness, efficiency and economic viability of the applied services, such as an EMS agencies operation.

The present embodiment's platforms synchronizes and inter-links all available health care systems within and out of a health care systems network. The platform optimizes health care systems' resources and allow a direct communication line for EMS and hospitals to expand and potentially provide treatment in the patients' homes instead of flooding hospitals. As an example, for EMS, the present embodiments' machine learning algorithms have the ability to re-train themselves with the real-time stream of data entering into the system on a daily basis, to provide more accurate predictions.

Although the present embodiments are shown within the EMS setting, it is noted that the present embodiments may be applied not only to other health care industries but also many other industries where variation in service demands strain resource management from time to time in seemingly unpredictable ways. The present embodiments add a level of prediction in such situations that is unknown in the art. In short, the present embodiments are not limited to human resource management. Other applications include supply chain management tools, interhospital and EMS-to-hospital communication platforms, predictive epidemiological modeling systems and the like.

Coupled with the predictive capabilities, the present embodiments becomes a centralized hub of integrated health care networks, by-passing the hurdles which currently inhibit the co-operation, and communication of EMS and hospitals. This concept is attractive to not only the private health care model within the US, but also to governmental-based healthcare models, such as in Europe, and other parts of the world. Regardless of the healthcare system, present embodiments are designed to offer solutions for every model.

The present embodiments are configured to be a centralized hub for management of healthcare, public services, and the like. It allows a health care system the ability to function at its maximal efficiency, even when unexpected circumstances create a situation where a minimal number of resources are available. For example, the present embodiments provide the ability for health care systems in even the most underdeveloped regions of the world to profit from all the advances made in health care. This allows technologies for hospitals to have the capabilities to not only provide a higher level of care, but the hospital also serve as a central hub of refuge in times of any kind of ecological or epidemiological crises.

Again, healthcare is not the only field subject to the challenges of intertwined large-scale operations that can vary over time. None of the public's services, including fire, and police departments, are immune to the effects of ineffective resource management. The machine learning based forecasting systems of the present embodiments, which leads to optimal resource utilization, provides countless benefit in many service industries outside of health care sector, such as construction, restaurants and the like.

To understand the present embodiments, an exemplary embodiment of a system to keep track of employees of a large organization is described within EMS services, where shifts need to be staffed sufficiently at any given time. Building on the predictive capabilities of the present embodiments, the scheduling module takes dispatching one step further by automatically adjusting the schedule of an agency based on expected call volumes in the region. The scheduling module automatically generates a suggested scheduling template which matches call volume appropriately to provide the maximum ambulance per call ratio per shift and is configured to configured to ensure maximum revenue for an EMS agency. The schedule suggestions become more accurate the more the system is trained. The system continuously re-trains itself in real-time. The scheduling module using cellular triangulation, GPS technology, and the like, and combinations thereof also makes suggestions to which stations an agency should staff, to decrease response times. During the day a dispatcher or EMS crew can check the system and gravitate towards calls, to decrease response time. The system also ‘fills in the gaps’ or ‘adjust’ itself automatically when EMS providers ‘call in sick’; or are planning to ‘take off’ and rearrange the shifts as needed based on future predictions.

Current scheduling software rely on deductive software only to produce schedules for their customers. The field of Emergency medical services, however, is very dynamic where agencies need to optimally account for variations in demand. The present scheduling module automates the process of scheduling staff and creates a schedule to maximize the ambulance:call ratio to maximize EMS agency reimbursement. The present embodiments learn from incident time, location, and type to predict and forecast future call volume type and location in real-time. Then adjusts a schedule template based on average call durations, time, location, and appropriate response unit to ensure a maximum ambulance:call ratio. The present embodiments re-trains itself in real-time and creates more accurate predictions, adjusting the schedule and forecasting models in real-time. Using machine-learning modeling, the longer it is used in an agency the more accurate it becomes.

Currently, EMS agencies rely on past statistics, and ‘gut’ feeling to distribute their staff accordingly. EMS agencies determine a pre-set number of ambulances on a given day which is limited based on what the agency can afford. Many days this number may be too low and on others too high, leaving EMS agencies over staffed on many days (losing money) and understaffed on other days (causing delays in patient care and losing calls to mutual aid). What the present embodiments offer is a tool that applies machine learning to predict future call type, volume, and location allowing EMS agencies to allocate their resources with far greater efficiency than previously known.

The present embodiments may apply calculations to its own continuously improved predictions, which then offer suggestions to the user to allocate ambulances accordingly. This results in an ambulance being able to respond to more calls and to cut down the time ambulances are spending on-duty but not responding to any calls. Thereby, EMS agencies' revenue is increased.

The present embodiments may apply primitive neural networks (see e.g., FIG. 26), and deep neural networks (see e.g., FIG. 27). Inputs from at least one or more data sources such as traffic conditions, weather, incident location of emergency or non-emergency call, call type, dispatch type, latitude and longitude of incident location, age, sex, chief complaint, incident date and time, holiday, day of the week, call classification, emergency department population status, incoming EMS service requests, and the like, and combinations thereof, are processed in real time to train the ML models, which provide predictive outputs of future events, supply usage and the like, for the user, and suggestions for the user to optimize performance based on the systems predictions.

The present embodiments are designed as a single tool, offering many use-cases for EMS administrators, Dispatchers, on-duty staff and can easily expand with in-hospital staff including physicians, administrators, and all healthcare providers.

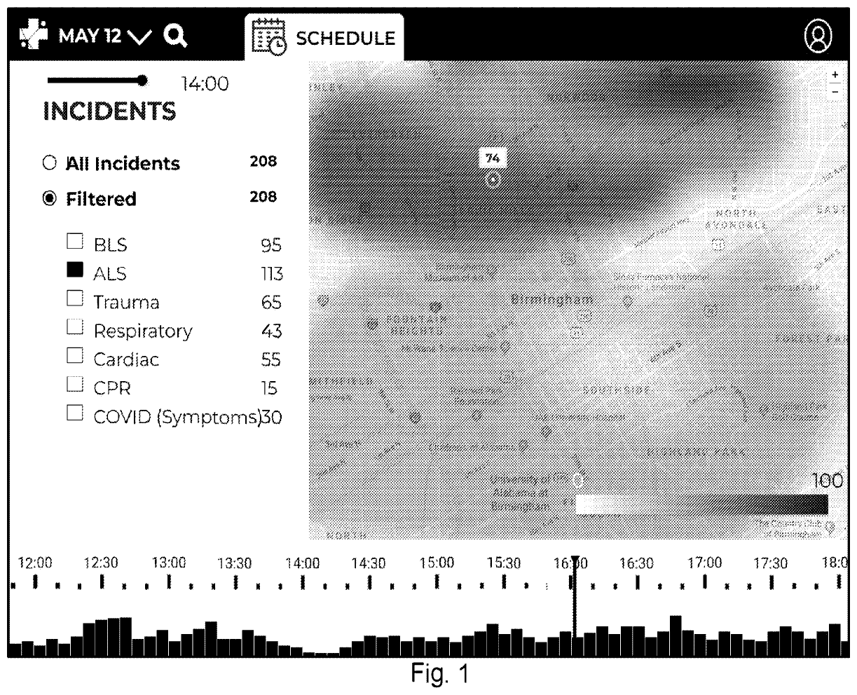

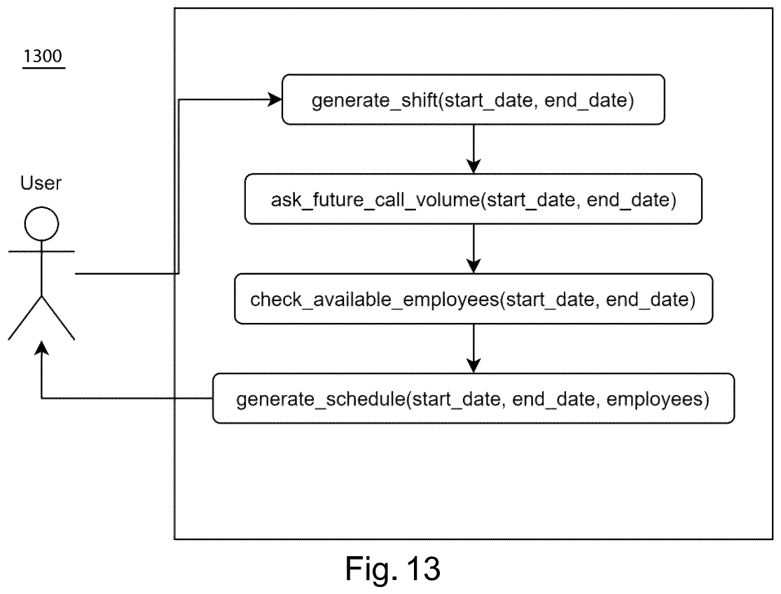

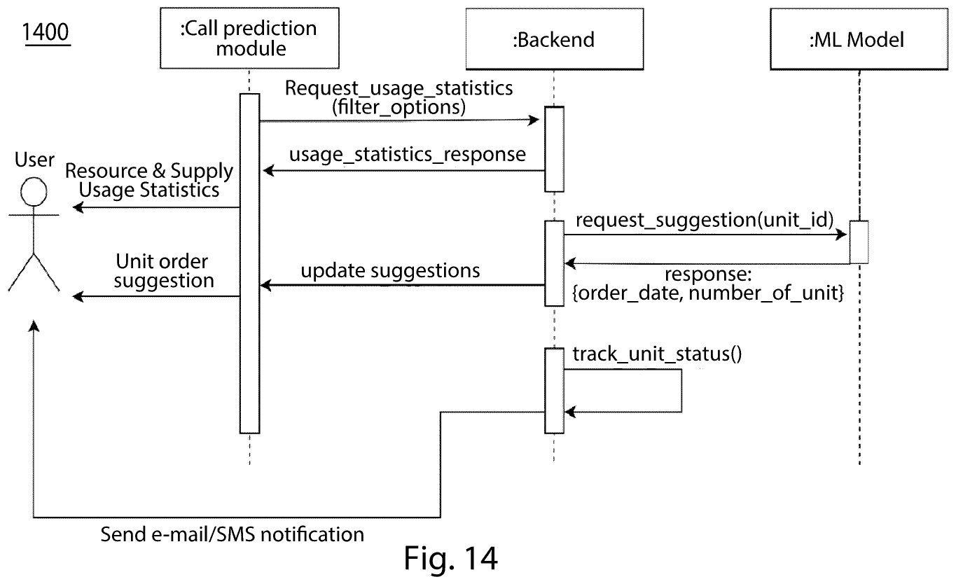

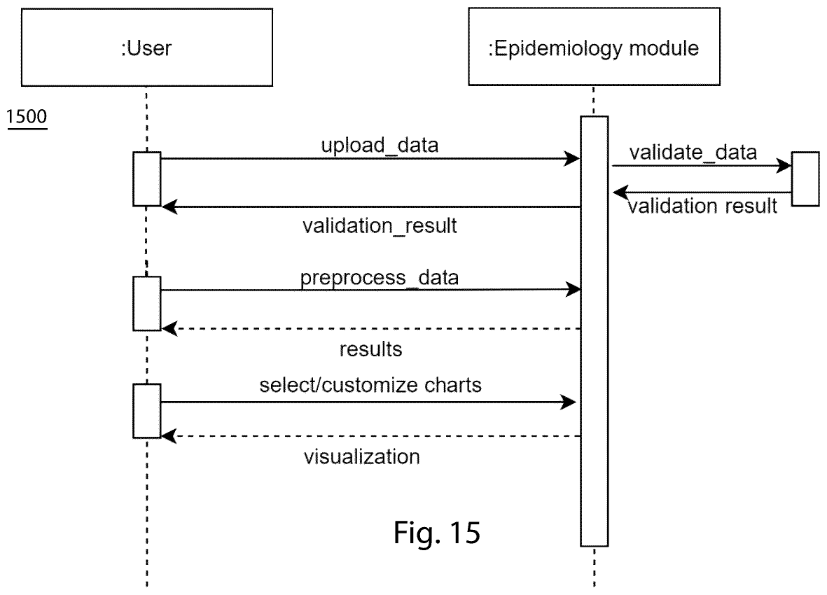

One key feature of the present embodiments is to predict future medical emergency demands using field-data around an institution and machine learning models (see e.g., FIGS. 26 and 27) to provide suggestions for given institution to operate more efficiently with the amount of resources it has at its disposal. Using data visualization techniques, the present embodiments may display large amounts of data on a single page in a user-friendly fashion see FIG. 1. By changing the focus on the input/output it provides a number of different applications, and use-cases. These include but are not limited to automatic scheduling see FIG. 13, tracking of epidemiological data see FIG. 15 for research, and resource/supply management see FIG. 14.

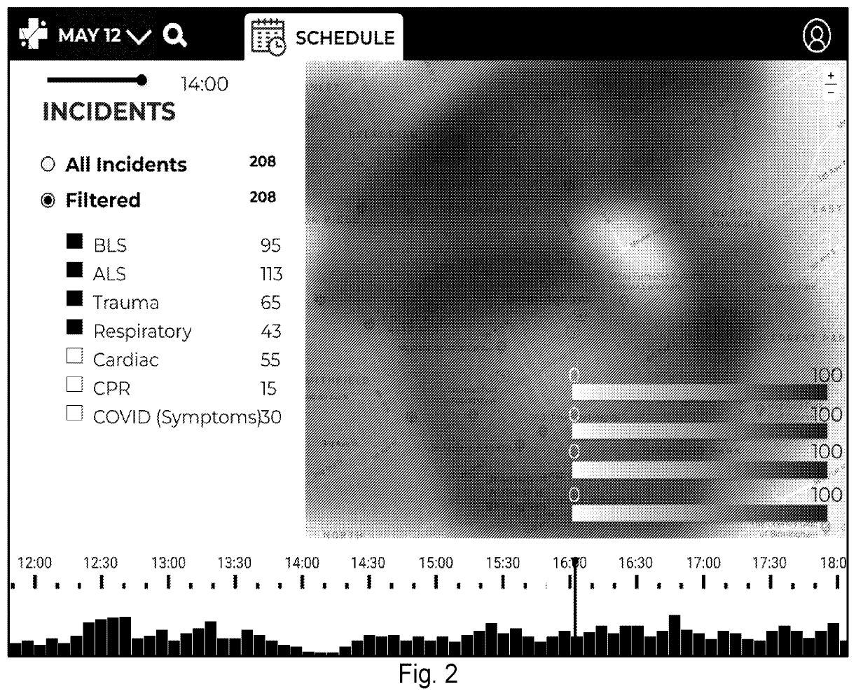

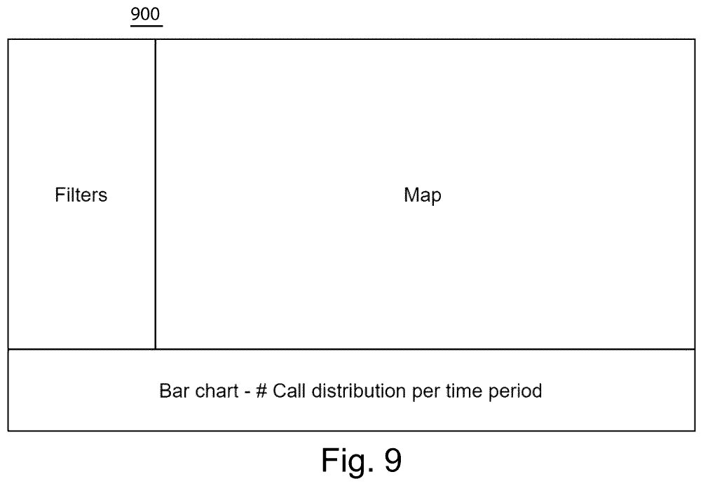

The present embodiments may be a cloud-based, software as a service application SaaS, meaning it can be accessed from any stationary or mobile device with an Internet connection. Key features of the embodiments include a maps view of the area EMS agencies operate in see FIG. 9 displaying predicted incident location and filter options to select for different incident types such as basic life support, advanced life support, trauma, and the like. See FIG. 2 Date and time may be chosen with a time bar on the bottom of the page. See FIG. 1. For example, see also below:

See FIG. 9. The map in the center depicts the region an agency operates in, or the surrounding area of the facility. The column on the left, shows different filter options for each incident, or call type. The time bar on the bottom displays the number of expected cases for the selected incident type. A cursor indicates the time which the incident predictions are shown.

See FIG. 2 Multiple filters can be selected, viewed, and compared simultaneously based on ML models, provided by the system. Scheduling adjustments may be made manually, based on the information provided. Suggestions are given from ML models, EMS units may be posted near projected emergency locations in real time, based on the predictions from the ML models. The system updates in real time based on the time and date the user has selected.

With these features EMS agencies have the ability to properly distribute their dispatch units across their sector to minimize 911 response times. Furthermore, agencies have the possibility to pull staff from times and locations where staff are needed less and redistribute staff to areas where staff are needed most. As a result, greater coverage is achieved with the same number of staff as door-to-ER transport times is kept at a minimum. Moreover, patient safety and work environment for EMS providers is improved.

Hospital staffing has been a key issue for many years. Changing dynamics in patient admittance combined with inappropriate staff utilization leads to excessive patient traffic in emergency departments. Hospitals often struggle to meet safe patient-to-staff ratios, which, if not met, can lead to a decrease in the quality of patient care. Patient satisfaction is heavily influenced by long waiting times during admission in the ER as well as discharges form the hospital. Fighting staff shortages is therefore a necessity to secure the quality of patient care and satisfaction, as well as a comfortable work environment for providers.

With the present embodiments' forecasting capabilities ER rooms can prepare for the expected patient influx, decreasing waiting time and increase work efficiency. The system also ‘fills in the gaps’ when nurses call in sick or are planning to take off.

Current scheduling software rely on deductive software only to produce schedules for their customers. Emergency medicine, however, it is a very dynamic field and hospitals need to consider the variations in patient influx, staffing nurses in the ER at all times. With the scheduling module of the present embodiments, hospitals have to possibility to move their staff to times of higher demand, taking advantage of the present embodiments' predictive capabilities, applying the same concept from the EMS applications to the hospital applications.

Hospitals struggle with high patient to staff ratios, overcrowding, and staff shortages. The present embodiments offer an all-in-one solution to optimize staff utilization suggestions, and auto-generate a schedule based on machine learning forecasting models in real-time. The present embodiments are a tool which apply machine learning, see FIGS. 26 and 27 to predict patient admittance duration in each department, and complaint type in the ER allowing hospitals to properly allocate their resources, in real-time and becomes more accurate the longer it is in use in an institution.

The present embodiments apply calculations to its own continuous predictions, which then offer suggestions to the user so to allocate staff accordingly. The busier a hospital is, the more patients, the more data means that the present embodiments retrains itself with more accurate predictions. The present embodiments is designed as a single tool, offering many use-cases including physicians, administrators, and all healthcare providers.

A key feature of the present embodiments' is to predict future medical emergencies using field-data around an institution and machine learning models (see e.g., FIGS. 26 and 27) to provide suggestions for a given institution to operate more efficiently with the amount of resources it has at its disposal. Using data visualization techniques, the present embodiments display large amounts of data on a single page in a user-friendly fashion. By changing the focus on the input/output it provides a number of different applications, and use-cases. These include, but are not limited to automatic scheduling, tracking of epidemiological data for research, and resource/supply management.

The present embodiments are a cloud-based application, meaning it can be accessed from any stationary or mobile device with an internet connection. Key features of the present embodiments include a maps view of the local area around a hospital. Quick and easy to understand charts of patient attendance in each department and type of complaints in the hospital. Department attendance can be seen in real-time and in the future. Quick comparison of busiest-to-slowest departments, allow for reassigning staff in different departments in present and future time if needed. For example, see below:

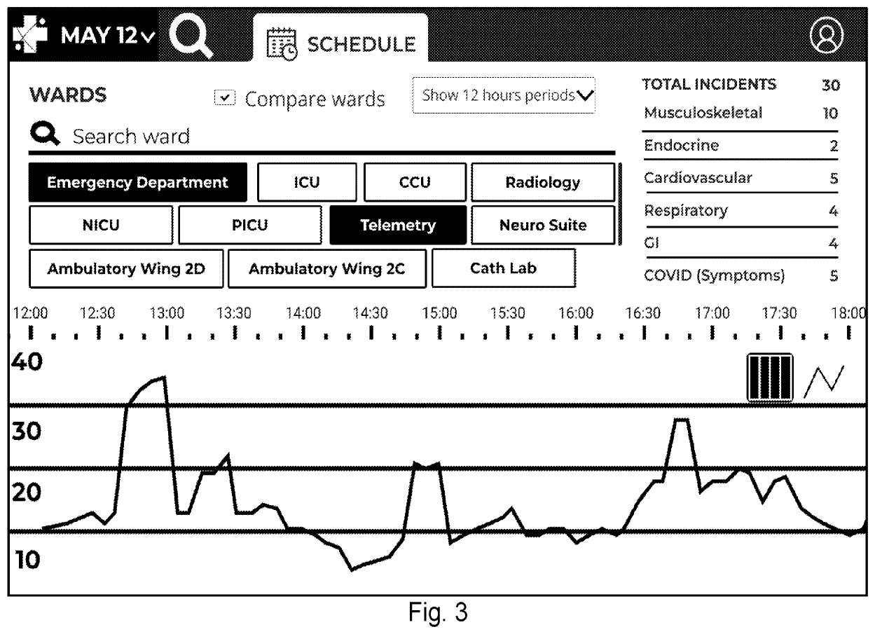

Single department ML forecasting. See FIG. 3. Departments can easily be selected, with graphic visualization of patient admittance and type at a specific time determined by the systems ML machine learning models.

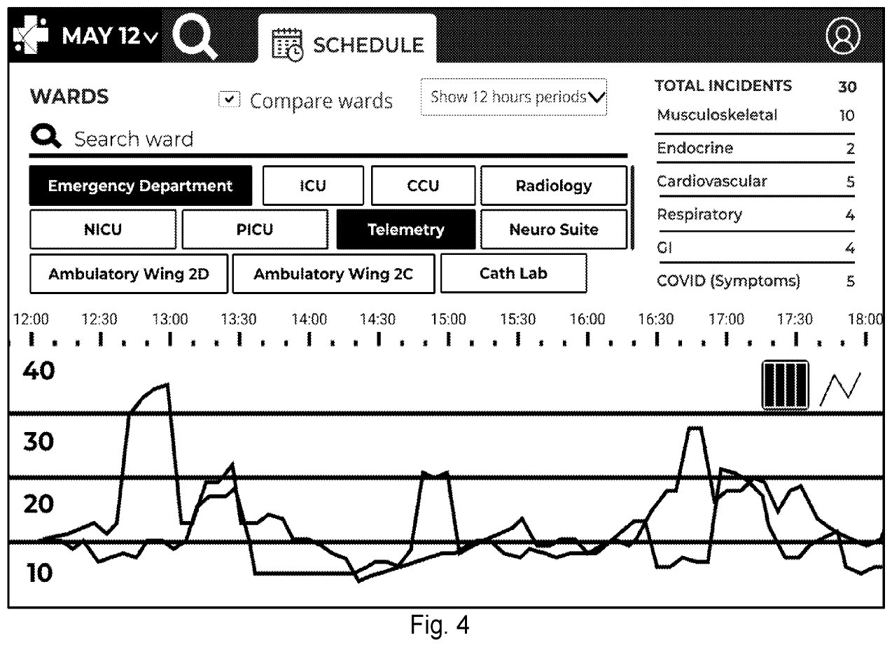

Two department ML forecasting. See FIG. 4. Two-departments can be selected and compare patient admittance and type simultaneously based on the systems ML models, for the added ability to move staff around different departments as needed. Organizations can introduce their own policies in maneuvering staff around different departments to meet safe patient:staff ratios.

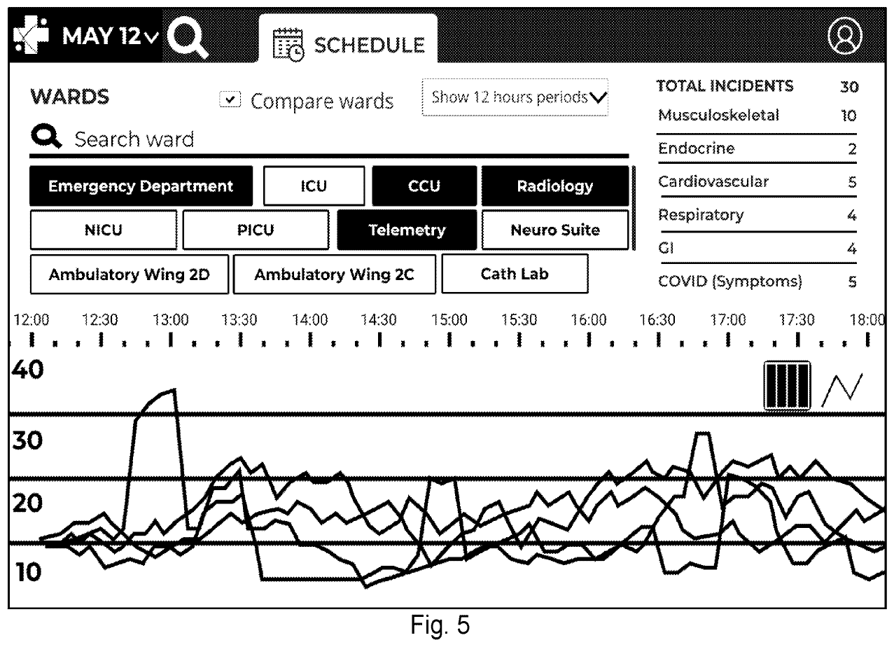

Multiple department ML forecasting, see FIG. 5. Multiple departments may be simultaneously selected and compare patient admittance and type in real-time and in the future based on the systems ML models. Suggestions may be made by the system or are manually decided to move staff around different departments as needed. Organizations can introduce their own policies in maneuvering staff to meet safe patient:staff ratios. In some embodiments, machine learning may be applied to track patterns of policies, manual inputs, decisions and the like to enhance and modify its suggestions in the future.

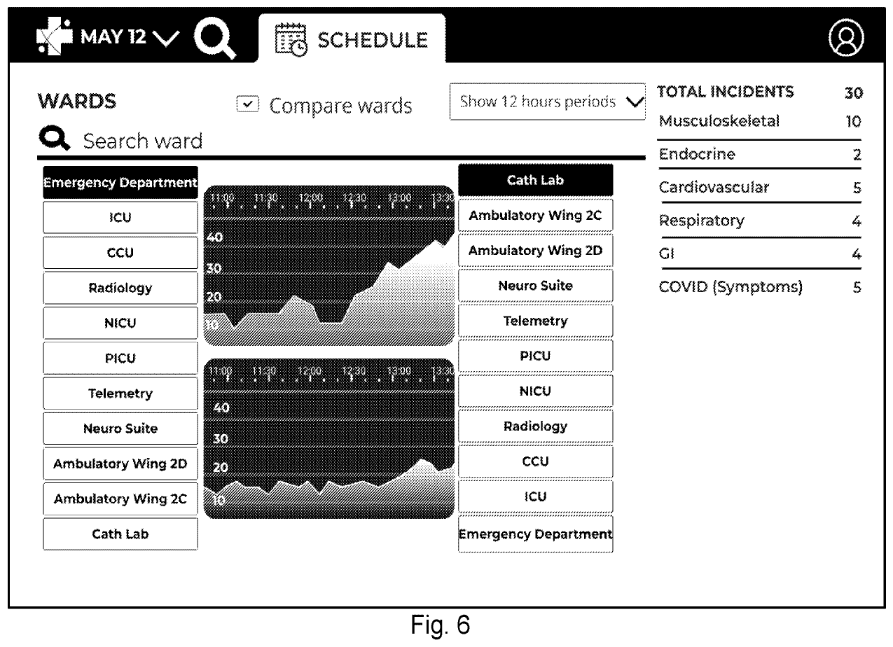

Rapid department assessment. See FIG. 6. The system automatically compares the busiest with slowest departments parallel to each other for quick rapid movement within the hospital as needed spontaneously. The user can move the time forward, and the system compares the departments based on the predicted census from the ML model predictions, to help prepare for future events.

In regard to resource and supply management, the present embodiments provide the ability to track and predict future supply usage, allowing health care systems to minimize waste, save on costs, and always be prepared.

For example, a lot of materials and medical consumables such as medication are wasted in EMS. Agencies a required to keep a full stock of medication at all times in order not to run out. Medication stock in particular is prone to waste as shelf life across various medication can vary, which required extensive monitoring.

The present embodiments track, predict, and suggest the appropriate number of units of medical consumables to order for a given supply and at the optimal time in advance. Over- and understocking are addressed by predicting future supply usage and making suggestions on the proper amount of how much to order. Minimizing waste while maximizing the utilization of existing resources helps reduce the financial burden of EMS agencies and result in improved patient outcomes.

In regard to hospitals, the category of inappropriate supply storage can be broken down to two categories: medical waste and supply shortages. Medical waste can be broken down into two categories: overstock and single-use items. Currently, hospitals are required to keep a full inventory at any time with often leads to overstocking and even wasting of used supplies and medications.

As with the EMS embodiments, the present embodiments track, predict, and suggest the appropriate number of units of an item to order for a given supply and at the optimal time in advance. Over- and understocking may be addressed by predicting future supply usage and making suggestions on the proper amount of how much to order. Optimal resource utilization helps reduce the massive cost of healthcare provision.

In regards to on-site manufacturing, supply chain logistics of materials and equipment are a critical component of the healthcare field and may be subject to failure in crisis situations. In the face of the covid-19 pandemic, many countries have experienced significant weaknesses in their supply chains as hospitals are struggling to meet their daily demands for medical consumables such as oxygen⁴. This led to many delays in patient care and drastically decreased patient safety. Moreover, medical supplies are often composed of un-recyclable plastics attributing to environmental waste. The amount of waste from medical supplies attributes to the rising cost in healthcare and health technologies. Medical products and consumables need to be shipped which leads to higher emissions a greater carbon footprint.

The present embodiments also envisions the production and recycling of medical equipment and devices on-site at the facility, utilizing 3D printing and medical equipment with interchangeable parts. Moreover, on-site manufacturing (OSM) is designed to produce mechanical or electronics devices with interchangeable components, which may be used for multiple purposed adding redundance into the system. Recycling used and unused components or materials on-site closes the supply loop, allowing for greater sustainability. On-site manufacturing (OSM) has the potential to alleviate the risk of dangerous supply shortages, while at the same time help improve disaster relief, if necessary. All these factors reduce the logistical burden of the healthcare sector and allow for faster relief in medical crises. Connecting OSM manufacturing with the predictive capabilities of the present embodiments allow for intelligent automated production of medical supplies at local facilities.

Accordingly, medical supplies can be broken down into two major categories: consumables (e.g., oxygen, medication) and non-consumables (e.g., single use masks, gloves, syringes, and the like). Both of these categories require a different approach. The production of consumables which are most often needed such as oxygen and saline drips may be anticipated with various existing solutions on the market today. For example, oxygen can be produced by either direct extraction from the atmosphere or by electrolysis of water. First generation portable oxygen concentrators (POC) have already been demonstrated, which have yielded a similar effectiveness in supplying oxygen to patients with respiratory diseases when compared to conventional oxygen cylinders⁵. However, there are challenges of scalability and performance such as electricity usage which may cause potential drawbacks. The production of medication and non-consumables on the other hand requires more sophisticated technologies and methods to be implementable in a hospital environment.

Additive manufacturing offers the potential to quickly produce devices and products that are most needed, such as syringes, injectors, parts for respirators and other medical technologies. Fabrication of tools and simple mechanical and electronic parts such as ultracapacitors have already been demonstrated successfully in extreme remote environments, immensely reducing the cost through the sole requirement of raw materials and components⁶. Complementing this on-site manufacturing approach with recycling of used or unused materials closes the loop, allowing for greater sustainability. A combination of a 3D printer and plastic recycler has been tested by NASA, the so-called ‘Refabricator’ which unlike to conventional recycling methods on earth does not require to grind its materials, thereby producing stronger filaments of higher quality from old, recycled parts⁶. Applying these technologies to the medical field could reduce the risk of supply shortages, thereby extending the scope of possibilities in providing medical care and disaster relief.

The fabrication of complex electronic parts and equipment on the other hand requires a different approach. A special resin which is used for example in additive manufacturing, that depending on its chemical manipulation during the manufacturing process, may act as conductor or insulator. This drastically reduces the steps and materials needed to produce electronic parts, simplifying implementation, and reducing cost of production.

Lowering the cost of printable, recyclable, environmental, friendly high-performance materials, have been achieved by using hydrogen bonds instead of, primary bonds (i.e., metallic, covalent, ionic).⁷ An example is a graphite-NFC based materials which exhibits a high tensile strength (up to 1.0 GPa) and toughness (up to 30.0 MJ/m3), greater than most materials such as steel, aluminum and the like.⁷ The development of printing metals, have been demonstrated to prove effective with magnetic liquid metal (Fe-Egaln) based materials.⁸ These metals offer flexibility, self-healing, and are at low cost.⁸ The sustainable production of graphene ink for wireless connectivity and IoT (Internet of Things) applications (i.e., mobile phones, laptops and the like) have proven successful with a non-toxic solvent Dihydrolevoglucosenone (Cyrene) based solution.⁹ Polymerization of pyrolle, and polymerization onto the surface of cellulose crystals have demonstrated to be effective in the development of homogenous composite materials composed of electrically conductive fillers and an insulating matrix.¹⁰

The material used in the embodiment of systems of OSM, is a high-performance compound, non-toxic, bio-degradable, low-cost material which has the properties to be printed, recycled, and bio-degradable. The material is used as the components for electrical based technological devices, and non-electrical devices via additive manufacturing and, or 3D printing techniques.

The material is a homogeneous substance, with electrically conductive fillers, and an insulating matrix. The material exists in solid state and liquid state. The material in solid state when made into a device can safely with stand temperatures of approximately ±60° C. The material exists in solid state at approximately between −50° C.-80° C.

In the event it is necessary to have a non-homogenous material, the same properties and requirements follow, except a single source material is a conductor and a separate single source material is an insulator. The two non-homogenous materials, separate through a natural process, or through a mechanical process.

In the event of consumables, separate source materials exist to ensure the safety and efficacy of the medications printed and produced through the OSM system.

This is a holistic approach meant to redesign medical equipment used in the hospital to be constructed out of interchangeable parts. This includes any mechanical device ranging from simple hospital stretcher or beds to treatment tools such as tubes and plastic valves used for draining wounds, fluid administration, mechanical respiration etc. For example, a saline flush may also act as a hinge joint for a hospital bed, a stretcher etc. This approach is crucial as it reduces the amount of part needed to construct, thereby decreasing complexity and adding redundancy in the supply chain.

Most medications are integrations or synthetic copies of existing enzymes, hormones, amino acids, etc. In the human organism, a single amino acid is the backbone of several hormones, following a chain of enzymatic reactions, and depending on which cell is being described, another enzyme may alter the amino acid in such a way to either create a hormone, and give the amino acid a completely different function or similar function with different affinity. For example, tyrosine is an amino acid which is modified to melatonin, dopamine, epinephrine, and norepinephrine.

Enzymes are activated or deactivated either naturally by the direction the reaction is pushed or by artificial means. For example, structural changes may occur within alternating a magnetic field or something less complicated like, temperature, acidity, etc. The body uses these constraints such as temperature and acidity already for enzyme activation or affinity.

Insulin is an example, which shows how science has already taken advantage of the ability to insert gene sequences into bacteria, to produce a certain protein, for example in this case, insulin. 3D printing and new additive manufacturing techniques are developing new methods to impact the pharmaceutical sector¹¹. The introduction of such methods and research are leading to the development of on-site medication manufacturing, which could be implemented using modeling of the present embodiments.

Developing a dispensary which is stocked by liquid bags filled with bacteria encoded with specific genetic sequences for the appropriate enzymes and prefilled with substrates that naturally produce a variety of the desired product. For example, tyrosine, having the bag pre-filled with L-tyrosine, and bacteria encoded with the enzymes in the tyrosine chain reaction can produce melatonin, dopamine, epinephrine, and norepinephrine. The on-site manufacturing of prefilling syringes with each medication allows for the development and administration of said medication. Enzymes is controlled artificially to ensure the production of only one product, or the products are sorted naturally by size, shape and affinity and charge.

The methodology in a step-by-step process is configurable to produce, the largest diversity of medications on-site regulated by the present embodiments. Starting with amino acids or any substrate which has a wide product range is the most effective way to start and open many use-cases.

The production of medications is regulated with the predictions made by the present embodiments through its forecasting models, predicting future spikes in diseases, spreading patterns of diseases etc. The enzymes also need to be regulated, to regulate the rate of production. The dispensary senses the level of the product in molar concentration and determine if it is appropriate to shut of the enzyme based on dose reached, or if production has already met the demand for the given day.

As healthcare services continue to increase in cost, reducing complexity and the amount of part in medical equipment is crucial to ensure the sustainability of the system. On-site manufacturing offers a new approach to the problem of supply chain logistics which the production of medical consumables and the development and maintenance of medical equipment.

In regard to EMS-to-Hospital communication, EMS often struggles to find proper communication with the local receiving hospitals to offer quicker, lifesaving, definitive treatment. A lack of appropriate communication modes hinders emergency departments from preparing accordingly to receive critical patients requiring immediate treatment. For example, in a situation where multiple systems trauma patients requiring trauma surgery, catheterization for STEMIs and TPA or thrombectomy for stroke patients, ER nurses and staff are forced to prioritize, which leads to ‘waiting lines’ in the ER. Communication is also typically one way from EMS to ED (Emergency Department). Communication should be two-way between EMS and ED, so EMS may appropriately distribute patients to more appropriate hospital destinations based on ED overcrowding.

The present embodiments' communication module provides a two-way communication route, allowing EMS crews to see ED population status, and offering EDs to plan and triage patients before their arrival. This module also provides early activation for trauma, stroke, STEMI, and MCIs, providing EDs time to prepare earlier than the current solutions currently offer.

The present embodiments provide an autonomous mode of transmission of patient diagnostics, 12-leads for the hospital to triage patients and prepare for either specialty treatment or create an open bed. This decreases the amount of ‘waiting time’ at the emergency room for EMS crew, providing multiple benefits to EMS-ER communication, as well as inter-facility and EMS communication.

In regard to hospital-to-hospital communication, hospitals, physician offices, and other patient caregivers face many struggles finding solutions to share patient information, such as diagnostics, to provide optimal and instant patient care. Currently, facilities use different documentation platforms and hold patient records in competing documentation platforms. There is not a HIPPA-secured method to share information between two out-of-hospital networks. Different hospital networks are continuously competing against one another, developing new methods for performing clinical procedures and protecting their methods under proprietary ownership, therefore discouraging the sharing of patient medical records of specific procedures performed to an out-of-hospital network.

The present embodiments' communication module incorporates all communication forms and share diagnostic information regardless of the physician or hospital network-providing a single communication network between facilities and streamline patient incorporation into new healthcare systems. The system offers a seamless and synchronous link between all users on its platform, harmonizing the ability to send diagnostic patient information, and offer quicker collaboration assessment and collaborative treatment. Patients benefit from the improved treatment regime as physicians have access to the full patient records, which reduces the risk of common treatment challenges such as drug-drug interactions with unknown or missed or over-prescription. Incorporation of patient into the naïve system is simplified.

In regard to Communication for Home Healthcare, including Community Paramedicine and other home health care servants, Community Paramedicine is a growing field that is in its infancy stage of development. When faced with the opportunity to nurture a new field in an optimal direction, the present embodiments provide the tools to reach the fields full untapped potential. Community Paramedicine is defined as specially trained paramedics who visit patients' homes to follow up a list of patients from a physician, recent discharges from a hospital or contracted by a medical alarm company to assist patients up from the floor if the patient fell and cannot get up. A community paramedic may be employed with hospice programs to visit hospice patients who require evaluation and comfort care. The role of a community paramedic is still in the infancy stages, which can branch into many critical roles that the healthcare system needs today, decreasing the burden of a healthcare system that is pending collapse.

Moreover, not every 911 call today is an emergency, and not every patient who goes to the hospital requires a hospital. Community paramedicine and other home healthcare programs offer the ability to assess patients at home, determining if there is a need for transportation or not. After a thorough assessment, the patient may be treated at home with IV fluids, antibiotics, or pain management. There are several goals of the community paramedic, each depending on its current role for the given patient. However, the primary benefit of a community paramedic is to provide first-hand treatment to patients at home instead of transporting them to hospitals or doctors' offices which decreases the overall burden of the healthcare system and can decrease re-admittance rates for hospitals. (This dramatically lowers Medicare/Medicaid reimbursement for hospitals).

The present embodiments aim to provide a direct communication line and share real-time diagnostic results, such as lab values, x-rays, or other imaging specified tests, between the patient's physician, specialist, hospital, and whoever is most appropriately determined. Whether there needs to be a collaborative discussion between the patient's physician and specialist, there is no limitation based on the ability to share diagnostic tests because of an out-of-network or time delays due to needing to wait to receive the results. Assessment, diagnostics, diagnosis, discussion, and treatment is performed instantly through the present embodiments' network.

The current limitation of community paramedicine is the inability for a wide range of pre-hospital diagnostics. Differential diagnoses are limited based on the provider's knowledge and diagnostic tests available to the provider. By increasing the ability for different pre-hospital diagnostics and imaging devices, in conjunction with a centralized communication platform, the present embodiments expand the capability and role of the community paramedic and other home health care providers to provide accurate field diagnosis creating a more beneficial impact, while further decreasing the need of transport to the hospital or doctor visits.

In regard to research and epidemiology, the present embodiments have the ability for the non-academic, epidemiologist, scientist, doctor, researcher to produce unique, customizable, user-friendly reports, charts, and predictions.

Epidemiological studies are essential in identifying the frequency of some instances or diseases in a localized region because it allows physicians to become more familiar with cases in their community. The more frequently the physician sees a particular case, the easier it is to recall this differential diagnosis in the future. Genetic mutations are an excellent example. These diseases are often misdiagnosed and mistreated in the field, which causes the patient to be sent back and forth to the hospital. The ability to compute the percentage and breakdown of the top 5 genetic mutations per region of each country would allow physicians to properly prepare and be familiar with performing physical exams and differential diagnoses for these genetic mutations, which have significant clinical findings. Every community has specialized needs but the same core problems. Providing a tool for users of all academic backgrounds to create individualized solutions for the information which is needed at that moment becomes crucial in developing a robust health care system that functions regardless of the challenges it faces.

The present embodiments' Research and Epidemiology module gives healthcare systems with the ability of epidemiological forecasting. This module enables non-academics, epidemiologists, scientists, doctors, and researchers enable to produce unique, customizable, user-friendly reports, charts, and predictions, including the ability to type the request in the search bar. Vital information is obtained by automatic report generation which allows providers to properly plan and prepare, improving their organization's workflow. For example, an emergency physician who would like to see the breakdown of cardiac myopathies or congenital heart defects in his city or state can quickly search for it while at work. Suppose an epidemiologist in a university hospital is tasked with tracking the different strains of flu. In that case, the user can do so quickly, simply producing the results in data visualization and friendly charts. The present embodiments provide the ability for each community to generate the information needed which has a positive significant of the healthcare systems. Using data visualization and color theory the module provides a user-friendly experience.

Currently, performing large-scale studies requires significant time and resources. Some epidemiological studies are performed based on systematic reviews, some based on extensive surveys requiring grant money. Sometimes, it is limited based on convincing a superior who does not share the same language or understanding its importance. This means a crucial idea may never come to fruition because of the lack of the ability to convey such information to someone who does not understand the field or problem. Because time is a scarce resource, only priority items get focused on. Even though there are items that may not meet the priority of the current time, it does not mean that the benefits and its rippling effect become less significant and beneficial for the impact on that health care system. Providing a simple platform that eliminates the need for grant applications, convincing a superior, and time-consuming, tedious systematic reviews and analysis gives the full benefits of a field study. This allows scientists and physicians to bypass common hurdles which are encountered in providing impactful studies.

Data visualization, such as those generated by the present embodiments, on the other hand, allows users from all educational backgrounds to analyze and understand large amounts of data instantly. The world now recognizes the importance of identifying epidemics and the potential spread of pandemics. The ability to track, monitor, and predict sudden changes becomes vital for the proper preparation of any health care system.

In regards to the present system architecture, the present embodiments AI utilize the following elements: Ensemble learning+neural networks, Decision tree and deep reinforcement learning via call simulations to create Model-free algorithms (Soft Actor-Critic, TD3, PPO).

Starting with simple classification and clustering algorithms such as k-nearest neighbor and k-means clustering implementation for the EMS call location prediction. While implementing these algorithms, literature review on algorithms for location prediction is performed.

Model-based algorithms are widely used in predictive uncertainty estimates for classification and regression problems. Ensemble methods are techniques that combines multiple models to improve accuracy. Ensemble methods yield the best results in number of machine learning competitions. Decision-trees are widely used in ensemble methods; however, any other predictive (classification/regression) models are used in ensemble methods. When neural network is used as a model in ensemble methods, it is called deep ensemble methods. The system implements ensemble and deep ensemble methods to predict number of calls and call types.

As described in herein the present embodiments use classification (supervised) or clustering (unsupervised) algorithms to predict call locations in the following hours. Deep ensemble methods are used for predictive uncertainty estimations of call types and number of calls. Each prediction happens in its own predictive model. Model estimates are combined to give the final predictive results for the following hours.

Present simulations to top of the base environment of OpenAI Gym. This simulation allows the system to run model-free Deep Reinforcement Learning algorithms. Model-free algorithms require more data and time to train, however, are prominently more robust to changes in the environment.

On the backend: Go is an open-source programming language which may be used to build the backend of the present embodiments. Go's concurrency mechanisms make it easy to write programs that get the most out of multicore and networked machines, while its novel type of system enables flexible and modular program construction. Go compiles quickly to machine code yet has the convenience of garbage collection and the power of run-time reflection. It's a fast, statically typed compiled language that is simple, reliable, and efficient to use.

### Orchestration Tool: Kubernetes (GCP)

On the frontend: Data visualization is programmed in Angular using D3.js. This allows for a better representation of data compared to regular table formats. D3.js is reactive, which means that instead of generating a static image on the server and presenting it to the client, it uses JavaScript to “draw” HTML elements onto the systems webpage. This makes D3 more powerful, but also a little harder to use than some other charting libraries. Angular on the other hand, is maintained by Google and is one of the most popular open-source front end web frame works used today. Exemplary modules are shown:

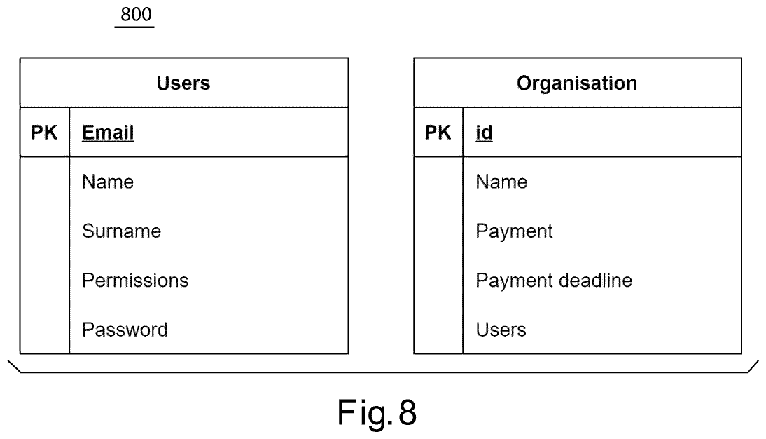

The present embodiments are a non-relational data base, representing the user and organization information needed for account creation see FIG. 8.

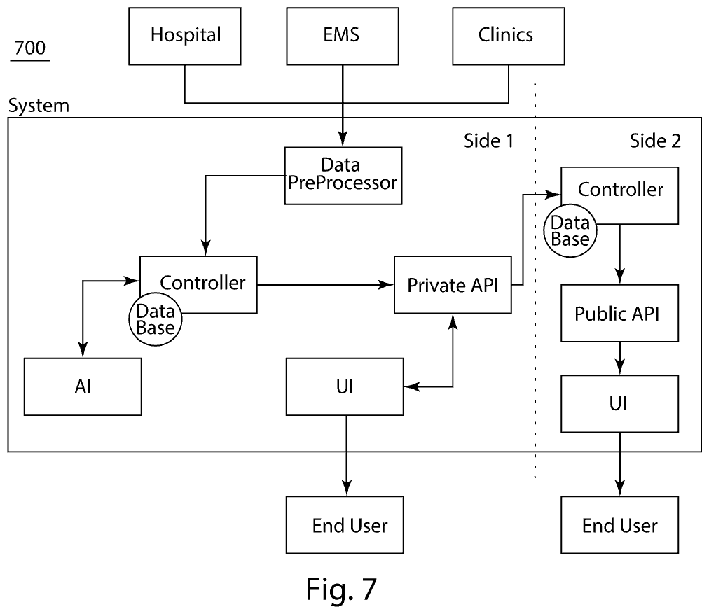

Main system data stream from input to ML machine learning to the end user. See FIG. 7. Side 1 represents the data stream if the facility chooses not to share the data for further research purposes as described in the research module. The data is “read” by the system and not collected. Facilities always have access to their own data to create research and epidemiological models based on their own data which the system receives from the local area. If the facility agrees to contribute to a larger data bank, for more accurate epidemiological and research models, the information the data flows through side 2, and the data is collected and stored meeting compliance regulatory standards.

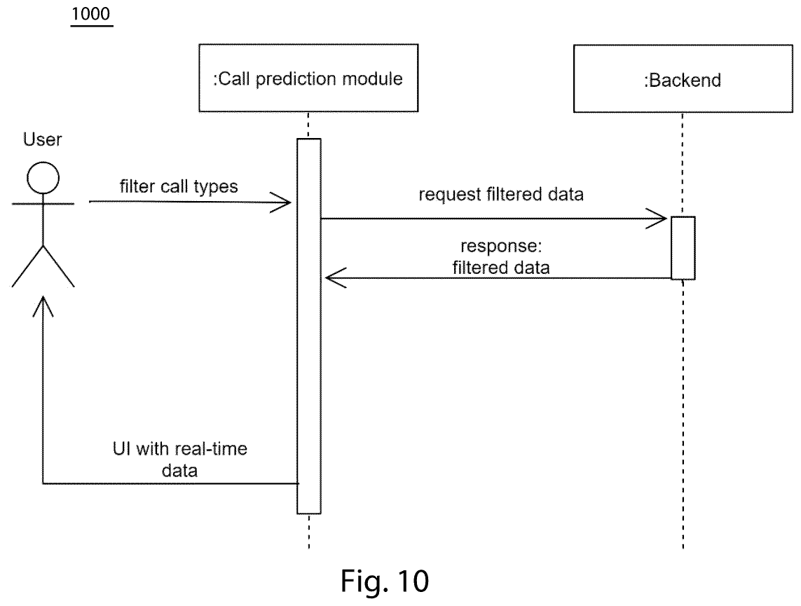

Core Module/Call-Prediction Module Architecture and Data Stream. See FIG. 10.

The call prediction module allows future call predictions (number of calls and locations for each time period—e.g., 13:00, 14:00, . . . ). Based on its predictions for a given date-time, a user visualizes call distribution on the map and draw a bar chart to visualize the number of calls day, while able to view in real-time past, present and future calls up to approximately 3 months at a time. The Call-Prediction module consists of two trained AI models, the one predicts the number of calls and other predicts the location of calls. Holiday input is a Boolean type (True or False).

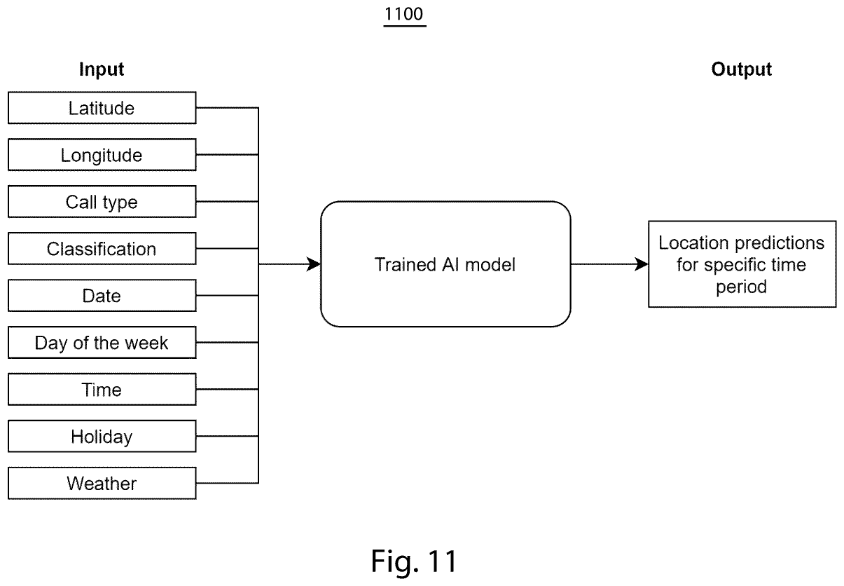

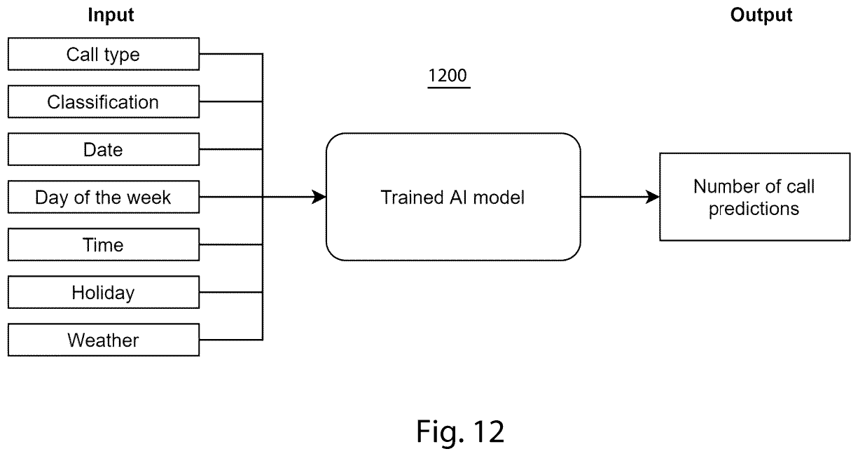

The core module presents the inputs for EMS call data in a user-friendly way in a form of a weather “type” map see FIG. 2 and FIG. 9. The inputs see FIG. 11 and FIG. 12 is processed in real-time by ML machine learning algorithms, and filtered by the user. The user is able to filter the call types which are desired, in real time and the probability of the specific selection chosen appears denser for the option selected represented as a color on the weather map. The less of a probable chance, the lighter the color appears.

The user is able to scroll in future time and the map changes in real time accordingly to meet the probably of likelihood for the selected filter types the user chooses. The user can change the filters ‘selected’ at any moment and predications are made and presented in real-time. Suggestions are presented in the map such as optimal ambulance location, specific locations of higher priority call types, and the like.

The field data inputs processed by ML machine learning is used to predict and make suggestions for ED (emergency department) admittances to the local area. Hospitals benefit from viewing call predictions around their facility to help better prepare for future events, and specialty patient types such as AMI's (active myocardial infarctions) CVA's (cerebral vascular accidents) and the like, in advance.

Scheduling Module. See FIG. 13.

The scheduling module takes the predictions from the ML machine learning algorithm and applies additional information to automatically generate scheduling templates to meet the optimal ambulance:call ratio when applied in EMS, and patient:staff ratio when applied in hospitals and facilities.

The scheduling module is designed to take ML predictions and rearrange the staff within an organization to meet patient demand. The scheduling module also automatically makes adjustments or suggestions in cases of “call-outs”, “vacation”, “sick-time”, and the like, based on the need of staff as determined by the predications made from the ML machine learning algorithms.

Resource and Supply Management Module. See FIG. 14.

Provides the ability to track and predict future supply usage, allowing health care systems to minimize waste, save on costs, and always be prepared. Backend tracks resource/supply status (number of units available) and sends notifications to the user when the number of units is below a pre-defined number. The user can access usage statistics for a specific unit or date period. Unit order suggestions are provided by a trained ML Model or a simple reinforcement learning agent.

When combined with on-site manufacturing, orders are sent to the on-site manufacturing device to produce the supplies needed in accordance to the demand, predicted from the ML Model.

Epidemiology Module. See FIG. 15.

The epidemiology module allows users to create custom data visualization diagrams an charts for research purposes. Users can upload their own data or also access the system's data collection, which is compiled from public APIs and sources (Kaggle, Data.gov, EUODP, . . . ). Users can generate different custom charts (bar, pie, line, bubble, box, . . . ).

Users can auto generate models and charts, based on the data output from the ML Models. Charts and diagrams can be generated by quick word searches and descriptions utilizing NLP to auto-generate a diagram to the users request. Templates of charts also be available and can easily modified for all user types. All user types of all backgrounds to be able to generate their own models, develop their own research and make organizational adjustments, and the like.

As regards to data: the core functionality of the present embodiments is its predictive algorithm which requires three datapoint categories initially: number of calls, locations, and type of calls. An exemplary data set below contains 5 columns: latitude, longitude, title, timestamp, and ALS/BLS type. Also, weather and traffic information is added. Increasing the dataset improves the present models' predictive accuracy.

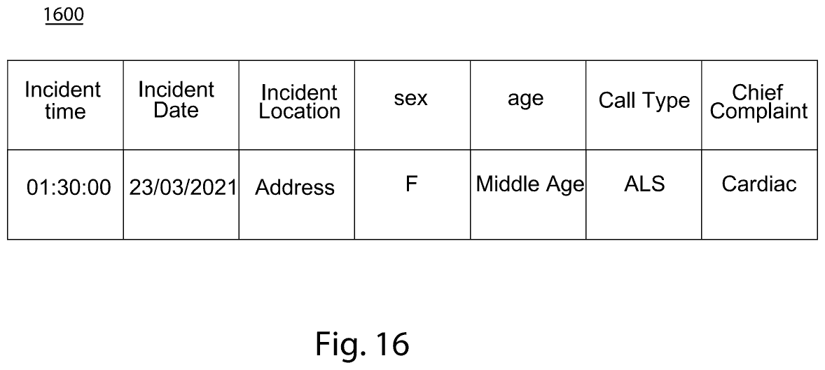

### End-User Data: EMS See FIG. 16.

Incident location: GPS coordinates are presented in the form of a ‘heatmap’. Depending on the amount of calls the intensity of the color changes. Variations in call types may be represented with different color codes.

Age: Age is stored as Newborn, Infant, Pediatric, Teen, Adult, Middle Aged, Geriatric The system takes the age and display it according to its corresponding value.

1-0 mo. Newborn
1 mo.-1 yr. Infant
1 yr.-12 yrs. Pediatric
13 yrs.-19 yrs. Teen
20 yrs.-44 yrs. Adult
45 yrs.-69 yrs. Middle Age
≥70 yrs. Geriatric

Call Type: Call Type is displayed in a generic form, for example: ALS, BLS or Pediatric.

Chief Complaint: Chief complaint is categorized as:

Trauma, Cardiac, Respiratory, Other Medical or CPR
For example, Chest Pain, is converted to and stored as Cardiac.
Difficulty breathing as “Respiratory”.
Fall as “trauma”
Unresponsive, diabetic as “other medical.

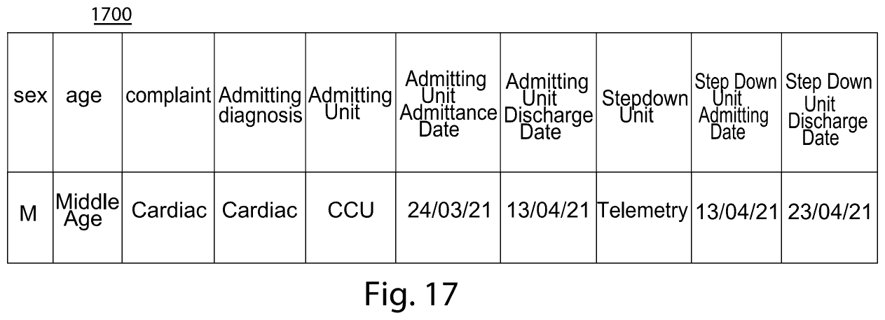

### End User: Hospital/Clinic See FIG. 17

Age: Age is stored as Newborn, Infant, Pediatric, Teen, Adult, Middle Aged, Geriatric. The system displays age of the patients according to its corresponding value:

1-0 month Newborn
1 mo.-1 yr. Infant
1 yr.-12 yrs. Pediatric
13 yrs.-19 yrs. Teen
20-44 Adult
45-69 Middle Age
≥70 geriatric

Admitting Diagnosis and Discharge Diagnosis: Admitting/Discharge Diagnosis is categorized as:

Trauma, Cardiac, Respiratory, Other Medical or CPR
For example, Chest Pain, is converted to and stored as Cardiac.
Difficulty breathing as “Respiratory”.
Fall as “trauma”
Unresponsive, diabetic as “other medical”

Data security and compliance: Following the European General Data Protection Regulation as well as the Health Insurance Portability and Accountability Act (HIPAA), the end user receives no permission to retrieve original datasets from the present embodiments. Furthermore, each call is categorized according to age and type of incident (shown above). The present embodiments infrastructure meets a high standard of software security and data protection.

Software implementation: The present embodiments are designed to run on existing client servers as well as on secure cloud servers. The present embodiments are containerized and separated from the customers main server system.

Each institution sends their data via the present embodiments' application programming interface (API). The user decides how to send their data (daily, weekly, monthly). This process can be autonomized if customers wish to do so. To allow for a pleasant user experience training options included to simplify the process of data transfer is provided.

The present embodiments (Controller container) is trained using received datasets, which are stored optionally in the systems database (DB) on a secure cloud server. End user interface is hosted on public cloud servers. After initial data processing, the End user receives continuous updates on call volume.

Pricing model: In today's market software products in health care are priced individually to the organization which is purchasing said product. This involves contacting the customer, contacting the seller, stating the organization size, number of employees, licenses needed, etc. The cost is based on predetermined calculations by the seller to determine the highest price for the software based on these parameters, which are derived from an estimate from predetermined calculations. This process also takes time and limits the scalability of the product and increases the cost on the seller's end, by requiring more customer service/sales employees.

The present embodiments and all of its applications and use cases goal, is to increase efficiency and scalability, while cutting down cost to the customer, and in turn the total expenditure on health care. Such system is incomplete without developing a pricing model which ensures and accomplishes such a goal.

The inspiration comes from how electricity is charged. A power plant harvests the product of electricity and then the consumer, turns a light switch on or off, and only pays for the amount of energy used, measured in kwh. Software uses energy, which can be measured. The present embodiments work in a similar way. Forecasting any future event such as a disease, medical case, 911 call, supplies, etc. requires computing power. The amount of computation changes based on the amount of data entering into the system. In other words, the more data, the more energy. Just like a light switch, if the light switch is on for longer, more energy. Turning more light switches on more energy.

However, data is always derived from a patient (or in other words) a customer of the user. To every patient there is linked reimbursement, in other words for data processing in, there is revenue made by the customer. The present embodiments forecasts future events, allocate facilities resources plan ahead and save money by utilizing its own resources, responding to more calls, decreasing admittance stays, therefore receiving more patients, decreasing waste from unused supplies which expire, and the like.

Therefore, it becomes possible to derive a formula, or multitude of formulas to create a universal pricing model for the software, which is based on the data being processed by our the systems, and user usage, which also ensures the consumer to save more than the cost of the product itself.

The system increases and measures the efficiency of the organization based on the ML Machine Learning predictions. The systems pricing calculator is directly proportional to the organizations improved total efficiency, accomplished by the systems ML Machine Learning predications. The better the ML models perform, the higher of an increase of efficiency for an organization, the more the system charges.

### EMS User-Case Pricing Model:

The pricing model of the present embodiments is based on charging a percentage of the increase of the average amount of calls one 1 ambulance responds to in hour. By lowering the average idle-time and increasing the call-volume for an EMS agency, an EMS agencies revenue is increased. The present embodiments charge based on the percentage of increased revenue for each EMS agency.

The present embodiments are a solution which saves the consumer more than the cost of the product itself. In order to understand how such a model is derived, the constraints, and variables, as realistic to the market as possible need to be understood.

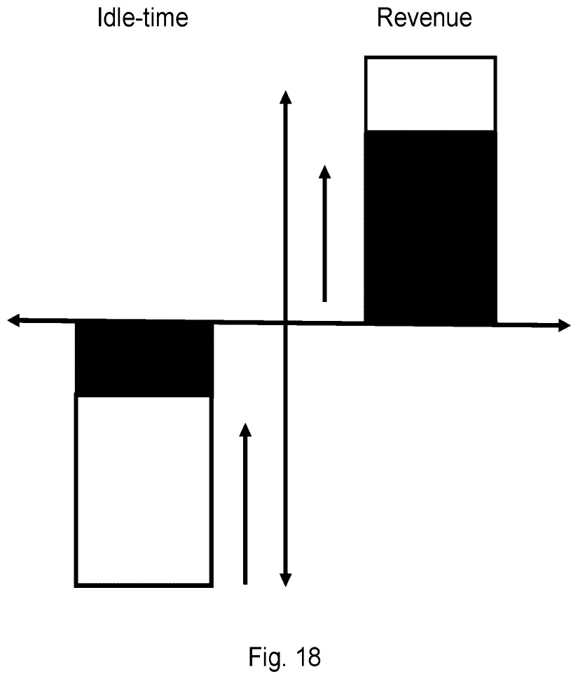

The following is a break down step-by-step approach, reviewing each variable form the pricing model to its goal. The graphic on the right dissects the two ways in which EMS agencies save on their own costs by using the present embodiments. (i) Decreasing the amount of time ambulances spend not doing calls (idle-time), and increasing the amount of calls an ambulance company can do, without hiring more staff (Revenue) see FIG. 18. The aim of the present embodiments is to decrease idle time to as close to theoretical 0 as possible, and to increase revenue as much as possible. First let's take a look at idle time.

Idle time is defined as:

$$i = b - z$$

i=idle time
b=true no. of ambulances/hr/agency
z=theoretical maximum no. of ambulances required/hr/agency

The present embodiments aim to bring the value i to as close to zero as possible, by decreasing b (the number of ambulances on duty at any given time).

Idle-time is understood, as the amount of time in one-hour an ambulance is not on a call. If an ambulance is not on a call, the company is spending money, on fuel, wear-and-tear, and salaries.

Essentially when an ambulance crew is not on a call, the agency is losing money. Revenue is only made from insurance reimbursement from calls, or if patients pay the bill, from patients themselves. This example does not factor in, maintenance, wear-and-tear, fuel and other costs businesses may face, such as utilities, etc. This example only looks at the salary of the ambulance crew. Other factors are not considered, such as private insurance reimbursement, and patients covering the remainder of the bill, federalgrants, tax money, and other sources of revenue these factors cancel each other out.

In order to understand these values, and the cost reflecting these values, lets dissect the cost of an ambulance shift in the steps that follow.

Step 1: Define a base salary: In order to create a formula, first the average salary for 1 ALS (advanced life support) ambulance consisting of 1 EMT, and Paramedic needs to be determined. This may change depending on the location in, e.g., NY state, and the kind of system. Sometimes an ALS ambulance may consist of 2 paramedics, and sometimes only 1 paramedic, which in EMS is referred to as a fly-car. But for simplicity, as it is most common to find 1 EMT and 1 Paramedic on an ALS unit, refer to the following example.

Average NY State Salary (which is on the Higher End of the Nation).

| | |
|---|---|
| AVG EMT Salary in NY | $32,512/year 15.63/hr |
| AVG Paramedic Salary in NY | $50,000/year $24/hr |

Taking the average salary, and comparing it to an average shift in EMS-12 hours, the cost of an average 12-hour shift for an ALS ambulance (1 paramedic and 1 EMT) can be determined

| | |
|---|---|
| EMT Salary | $187.56 |
| Paramedic Salary | +$288.00 |
| Cost for just salaries | $475.56 |
| in a 12-hour shift | |

Step 2 Define revenue: In EMS revenue is made based on billing insurance, private and Medicaid/Medicare, grants, and tax. It is important too not that this is not uniform. Not all EMS agencies are eligible for tax money, not all counties pay money for their local EMS service. Grants are not a reliable or consistent flow of revenue and are more geared if an agency needs to purchase a new item or ambulance. Medicare/Medicaid sets the standard for the reimbursement rate for private health insurance. Depending on the demographic area the ratio of patients with private insurance and public insurance varies greatly. Most patients have Medicaid/Medicare. Medicaid/Medicare reimbursement rates vary based on area, weather the call is ALS or BLS, and other factors, however for simplicity, the NY state of average of $275 is used.

“Under Part B of the federal Medicare program, only a portion of the cost of care is paid, leaving the remainder to be paid through beneficiary cost-sharing in the form of deductibles and co-payments. Generally, Medicare pays 80% of the approved amount for covered services. Currently. Medicaid pays remaining 20% for the deductibles and co-payments for “dual-eligibles”, those beneficiaries eligible for both Medicare and Medicaid. Currently ambulance service is a covered service for the Medicaid “crossover” reimbursement payments for dual-eligibles, low-income and elderly New Yorkers. The Executive has proposed elimination of the crossover payments for ambulance providers.”

“Eleven upstate and twelve downstate commercial ambulance providers worked with The Moran Company to study Medicaid Rate Adequacy and service costs. The upstate study found the average operating cost to provide ambulance service in urban areas is $304, in rural areas it is $543. The Medicaid reimbursements rates range from $105 to S190 depending on county. The downstate report showed the average cost per transport to be $281-$308 with Medicaid reimbursing only $155 for BLS and $200 for ALS calls. The results are clear, Medicaid rates do not come close to covering the cost of providing ambulance service.”

“The Department of Health released the Medicaid Ambulance Rate Adequacy Report in March 2017. It estimated the mean cost of ambulance trips to be $304 upstate and $247 downstate, for a statewide mean of $275.50. DOH recognized that the cost of providing services is higher upstate ($304) than downstate (5247) this is primarily due to the cost of readiness. It is more expensive to be ready 24/7/365 for 911 emergency medical service versus scheduled non-emergency interfacility transports. Yet this statewide average does not accurately represent the variance in cost structure between predominately 911 emergency providers and non-emergency inter-facility providers. The average creates a situation where Medicaid reimburses 911 providers below the mean and reimburse non-emergency providers above the mean.” (United New York Ambulance Network Budget Memorandum Executive Budget Proposal 2019-20).

In EMS revenue is made, only when a call is responded to, insurance reimburses, and patients are able to afford the remaining costs. Not all emergencies are billable calls as well. Some are call cancels, refusals of transports, and sometimes the patient doesn't have any insurance. However, these factors cancel themselves out based on private insurance reimbursements, which were not factored. In other words, a formula for revenue can defined as: Medicaid Reimbursement×No. of Calls/year=Revenue/year.

Step 3: Maximal No. of Calls/12-hour period. Breaking down a 12-hour shift. In EMS, there is no control of when people call 911, there are periods where no one calls, and periods where too many people call. It is also important to understand that one ambulance can only respond to one call at a given moment. This being said, the maximum number of calls one ambulance can respond to in one shift, and the minimum number of calls one ambulance can respond to in one shift needs to be determined.

An EMS call on average can last a duration of 1-2 hours. Splitting the difference and assume the average EMS 911 call lasts 1.5 hours.

| | |
|---|---|
| Hours in an avg. shift | 12 |
| Avg. duration of emergency call form | ÷1.5 |
| dispatch to back in service | |
| Maximal No. of calls in a 12-hour shift | 8 |

Under these constraints, in a 12-hour shift the theoretical maximal amount of calls an ambulance crew can respond to is 8.

Step 4: So far, Revenue, Cost/per 12 hour shift, and theoretical maximal number of calls one ambulance can respond to in one hour are defined. The next step is to combine these factors.

For the final formula, everything is measured in 1 hr. This makes more sense later. The theoretical maximum also needs to be determined for the number of calls 1 ambulance can respond to in 1 hour, the theoretical maximum is defined as X. In order to determine X in hours, divide 1 ambulance by the number of hours it takes for 1 ambulance to do 1 call. For this example, the average of 1.5 hours is used.

$$x = 1 / t$$

### Maximum Number of Calls Per Hour:

X=theoretical maximum number of calls 1 ambulance can respond to in 1 hour
1=1 ambulance
t=mean of total call duration/hour

Looking at this formula, for revenue one limitation of revenue is how many calls an ambulance can respond to in a given shift. In other words, if this number increases, revenue increases. By decreasing t, X can increase. Increasing X, increases the number of calls an agency can respond to, resulting in greater total Revenue.

### Example

$$\begin{matrix}X = 1 / 1.5 \\ X = 0. 6667\end{matrix}$$

X is the first constant, representing a theoretical maximal number of calls one ambulance can respond to in one hour.

### Step 5: Understanding ‘Z’, the Agencies Theoretical Maximum Number of Ambulances Required/hr

Z=Theoretical maximum no. of ambulances required/agency/Hr
A=Actual number of calls/Hr
X=Theoretical maximum number of calls 1 ambulance can respond to in 1-hour

$$z = \frac{a}{x}$$

Understanding ‘Z’ the agencies theoretical maximum number of ambulances required/hr, the maximum number of ambulances an EMS agency is required to have on shift, can be determined, in order to obtain a perfect 1 ambulance/1 call/1 hour ratio. This maximizes revenue to a theoretical maximum, and minimizing idle-time to a theoretical zero.

Step 6 Maximal Gross Income: By using the formula for revenue, it is possible to calculate the maximal amount of revenue.

| | |
|---|---|
| Example: Medicaid | $275 |
| Reimbursement × No. of calls = | |
| Revenue | ×8 |
| | $2,200.00 |

### Maximal Gross Revenue/12-Hour Shift

Maximal Gross Income: Now, by taking the value of maximal gross revenue, assume the net profit for a 12-hour day under the following assumption:

| | |
|---|---|
| Maximal Gross Revenue | $2,200.00 |
| Cost of a 12-hour shift | −$475.56 |
| Maximal Net | $1,724.44 |
| Profit/12-hour shift | |

### Example

In a 12-hour shift with 0 calls, the
EMS agency loses at least $475.56.

| | |
|---|---|
| Revenue | $0 |
| Cost of 12-hour shift | −$475.56 |
| Maximal Net | −$475.56 |
| Profit/12-hour shift | |

### Values Per Hour.

Having established maximal and minimal values for a 12-hour period, let's take a look at this value per hour.

### Maximal Net Profit/Hour.

| | |
|---|---|
| Maximal Net Profit/12-hours | $1,724.00 |
| Hours | ÷12 |
| Maximal Net Profit/hour | $143.70 |

### Minimal Net Profit/Hour.

| | |
|---|---|
| Minimal Net Profit/12-hours | −$475.56 |
| Hours | ÷12 |
| Minimal Net Profit/hour | −$39.63 |

Maximal No. of Emergency calls 1 ambulance can respond to per year. Earlier, it was determined that the maximal number of emergency calls 1 ambulance can respond to in a 12-hour period is 8, under the assumption that each call takes 1.5 hours. Under this assumption, one ambulance can respond to a maximum 5,840 calls/year.

| | |
|---|---|
| No. of calls in a 24 hour period | 16 |
| Days/Year | ×365 |
| Maximal No. of calls/year | 5,840 |
| Revenue | ×$275 |
| Maximal Revenue/year/1 ALS | $1,606,000 |
| Ambulance | |

It can now be assumed that, if an ambulance responds to 0.6667 calls/h the agency made a maximum net profit of $143.70/h. In other words, X=theoretical maximal revenue/hr.
0.6667 calls/h=$143.70/hr
0.182 calls/h=$0
0 Calls/h=−$39.63/hr

Applying the present embodiments. The theoretical maximal amount of calls a single ambulance can respond to is determined, given the average call time is 1.5 hours is 0.667 calls an hour. Unfortunately, in real-life calls vary in time, and in frequency. For example, 3 or 4 emergencies can come at once, and periods where there are no calls. There are two factors that the present embodiments are designed to increase efficiency. (i) Decreasing the amount on ‘down-time’ ambulances have to lose money in pay roll, and (ii) increase the amount of calls an agency is able to respond to.

To breakdown point (i) Decreasing the amount on ‘down-time’ ambulances have to lose money in pay roll. On average a company which may cover 4,500 calls a year have 2 ambulances on 24/7 (if an agency is able to fill all the shifts, which often is not the case). 4,5000 calls/year means the agency covers 0.51 calls/hr, and requires 0.76 ambulances per hour to reach is theoretical maximum. This agency is 1.24 ambulances/hr over the recommended 0.76.

2
−0.76
1.24

Assuming the cost from the estimated salaries from the beginning in a 12-hour period $475.56. The avg. cost of salaries per hour is $39.63.

$475.56
÷12
$39.63

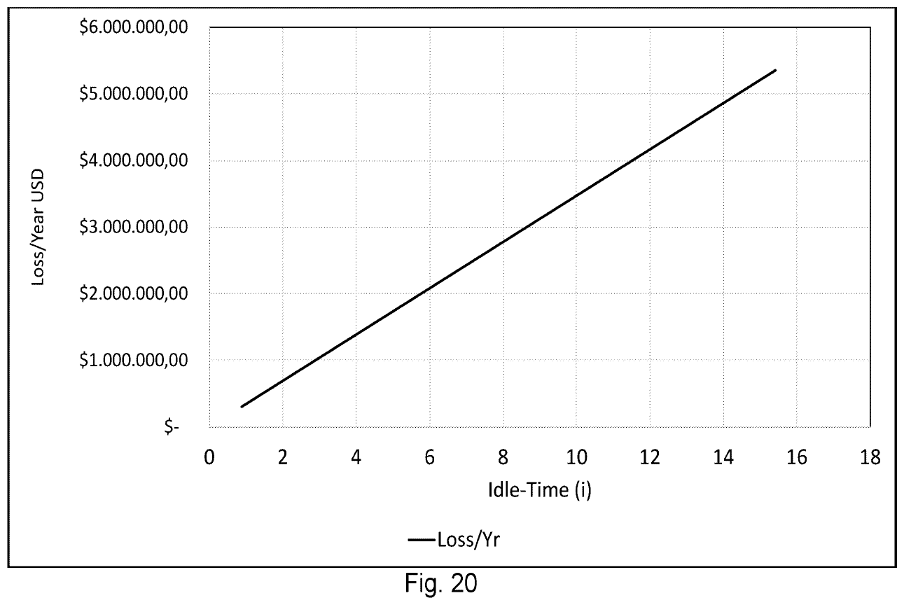

See FIG. 20. & 21. This agency loses a theoretical value of $49.14 per hour.

| | |
|---|---|
| | $39.63 |
| | ×1.24 |
| | $49.14 |
| Totaling an annual loss | |
| of $430,466.40 | |
| Loss per hour | $49.14 |
| Hours per year | ×8760 |

### Total Annual Loss

Step 7 (final) Returning to original formula for idle-time.

$$i = b - z$$

i=idle-time
b=true number of ambulances/hr/agency
z=theoretical maximum no. of ambulances required/hr/agency
   By predicting future call location, and time, the present embodiments can allocate the most appropriate number of ambulance units, to decrease the amount of ‘idle-time’ to as close to a theoretical zero as possible.

The present embodiments can measure the agencies i(initial) at the beginning of the month and determine the i at the end of the month which is called iα. The ratio between i:iα determines the amount of improvement as a percentage. This percentage represents the increase efficiency for idle-time which is called alpha a. If there is a 40% increase of efficiency in idle-time, this is written as α40%.

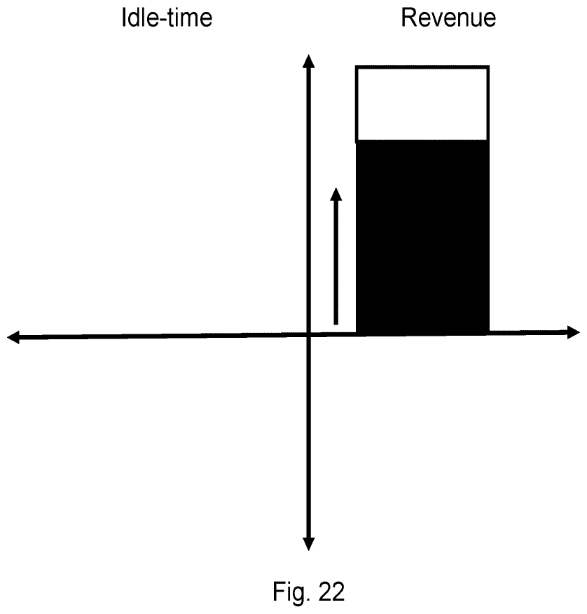

Revenue. See FIG. 22

The original formula for Revenue, stays as follows:

Medicaid Reimbursement×No. of Calls/year=Revenue/year

So logically, if the number of calls an ambulance agency is able to respond to per year is increased, agencies revenue can also increase. This can also be measured as a percentage, in a form of increase efficiency.

For example, if an agency responds to 5,000 calls/year, and with the present embodiments, the agency were able to respond to 6,000. That is 1,000 more calls in one year, a 20% increase in efficiency. Which is written as Ω 20%.

In a testing phase, the number of calls an agency responds to at the start of the month C is compared to the number of the calls at the end of the month CΩ, to determine the percentage of calls, an agency was able to respond more too in one month, C:CΩ.

Tying it all together. By adding the improved efficiency for idle-time and revenue, the agencies total improved efficiency is defined, which is represented as Rζ.

$$α + Ω = {R}_{ζ}$$

$$α = total increase of efficiency of idle - time$$

$$Ω = total increase of efficiency of Revenue$$

$$R ζ = total increase of efficiency$$

$$α 40 \% + Ω 20 \% = R ζ 60 \% total efficiency$$

Defining an agencies total improved efficiency, opens the avenue for a new pricing model. For example, if an EMS agencies total efficiency improves by 20% and a commission of 20% can be taken of their win. For example, an EMS agency which does 4,500 EMS calls/year and has 2 ambulances on 24/7, if it has a total improved efficiency of 20%, the present embodiments take a commission of 20% (which is 4% of the total number of calls/year×avg. Medicaid reimbursement). 4,500×275=$1,237,500×0.04=$49,500/year.

There are on average 21,500 EMS agencies in the United States. That is a very small market size, by creating a new pricing model based on number of EMS calls/year increases market size to about 100 million, which includes, 911 calls+private emergencies+interfacility transports.

The present embodiments were to develop a solution, which turns around a failing industry, and develop a universal pricing model which takes a percentage of the profits. This ensures that the system is charging based on a company's success, and in doing so took a global market size of $19.38 billion and turned it into a market size of 422.07 billion (7.674 billion*0.2*275). The total number of EMS calls relative to the US population is about 30%, if there is about 20% of EMS calls relative to the global population assumed, the global market size to be 422.07 billion charging a 6% commission.

Hospital User-Case pricing model. Applying the same concept from EMS, for in-hospital use, the present embodiments apply a logarithmic formula which charges the customer based on the amount of data processed ensuring a universal equation for any size hospital. A pricing calculator is connected to each customer, allowing a live cost and predictive future cost for the present embodiments program. Expansion to other modules requires more data being processed, and more usage which therefore increases the price, of the program, however, assists the hospital in other uses to save more money, or work more efficiently.

Diagnostic equipment user-case pricing model. The present embodiments also allow for other diagnostic equipment for the health care field (see diagnostic equipment), with the goal of making the devices portable, while increasing the use case of each device while introducing the ability to provide in-hospital treatment in the pre-hospital environment, with the goal of decreasing patient admittances and admittance stays decreasing health care expenditure.

However, the present embodiments offer a new model of charging for each product. Some companies have moved to a subscription-based system for example, butterfly network with the use of USG. The present embodiments take this one step further and develop a formula to charge based on usage of the device, hardware and software usage is measured, in computing power, or kwH, etc. A pricing calculator based on the time of usage of diagnostic devices.

The pricing calculator of the present embodiments ensures the customer is saving more than the cost of system. The present embodiments pricing calculator is dependent on the predictive algorithms, to ensure proper usage of resources, supplies, staff, etc which ensures the how the customer saves on expenses. If a new product is released, and shows how it saves the customer money, the way this is calculated is by a clinical trial, prior to the purchase of said item. This is not a universal savings model because how much a facility saves is dependent by the size, number of patients, operations etc. The present embodiment's pricing calculator shows the customer real-time cost and usage, and by changing the price to data input, this ensures that it is the same proportion for any size facility, EMS agency or customer. Smaller facilities see less patients than larger facilities. Urban facilities see more patients than rural facilities, etc. With a real-time window showing the customer how much they are spending, the present embodiments also shows predictions of cost based on the systems machine learning forecasting. Linking this concept to medical devices, creates a single source payment for a customer, along with ensuring the customer is only making expenses when itis using the product and receiving reimbursement for the product. Creating a win-win scenario for the customer. This method also removes the need for customer A to contact seller A to receive an estimate, based on company size, number of user accounts, number of licenses, etc, for then seller A to recontact customer A with a quote which either is favorable to the customer and the seller loses on profit or non-favorable to the customer and the customer is overpaying. This ensures the savings for the customer and maximizes the profit for the seller, while still lowering the overall cost of health care expenditure, and assists turning a failing industry EMS around which is the backbone of a health care system and is the corner stone to drive in-hospital care to the home, which is the direction the medical industry is currently recognizing is needed to go to sustain itself.

Bringing the present embodiments into the physical world. Today the health care setting often experiences many solutions which are containerized separately, creating a delay in patient care, documentation errors, and exploits the ability for institutions to spend more to create “solutions” to these problems. Every health care system is a spider web of many moving complexities. The present embodiments tends to string together such complexities and automate processes, streamline communication, and information, while automating documentation for health care professionals.

Due to rising costs in health care, there has been a push forward to decrease hospital admittances and decrease hospital admittance stays. Different programs, such as community paramedicine, telemedicine and visiting home nurse services have been moving in this direction, limited to the technological resources which allow these fields to flourish to their maximal potential. Before the topic of how to connect a multitude of devices from prehospital diagnostics, in-hospital diagnostics, and documentation, from different hospital networks, the information needs to be processed and sorted.

The solution of the present embodiments is a “sorting” and “packaging-center”, for all devices, ranging from prehospital diagnostics, in-hospital diagnostics, and documentation, with-in different hospital networks. The present embodiments have the ability to connect with any device regardless of if it is in the system family or not. The present embodiments utilize a smart system which reads another systems syntax and automatically connect it to the system's API. This makes the process of connecting APIs autonomous. Often when problems exist in connecting API's it needs to be manually fixed.

The present embodiments can read, interpret, and sort the data to automate documentation, billing, compliance, and patient reports at patient hand-offs. The following show how the present embodiments work when dealing with its different components.

Pre-hospital diagnostics. The advancements of diagnostics have led the ability for software to be incorporated into diagnostic imaging devices, a tool to assist providers in making more accurate diagnostics. This includes providing differential diagnostics, measurements, and suggested treatment, etc. Several companies such as Butterfly network and Brain scan have applied these methods with the use of artificial intelligence-based programs for the use of ultrasonography and CT imaging. The present embodiments provide a multi-functional portable imaging devices to be part of the system (see diagnostic equipment). Medical devices inside or outside of the system have the ability to connect automatically to the present embodiments to be sorted for a number of uses. The pertinent information which is required in documentation for the respected health care professional, paramedic, nurse, doctor etc. are inserted automatically, and information is sorted and automated for multiple use cases, including—to the appropriate slot in the medical report, back to the machine learning algorithms of the present embodiments in real-time to make more accurate predictions, and offer real-time data for scientists, epidemiologists, and other users to use in the data visualization library.

The following examples provide specific use-cases as an example which is applied in a multitude number of medical emergency cases and uses. For simplicity's sake in each example, one specific medical emergency is applied. The term system or the present embodiments, is used to describe a software-based system which utilizes NLP Natural Language Processing and ML Machine Learning to extract, sort, prioritize, and send data within a system to interconnect multiple systems. This process includes automatic packaging and sorting patient data for and between EMS and Hospitals, automating and synchronizing the documentation process, automatic and simplified API connection between different software and hardware providers, and the like.

Example 1. Use case of the present embodiments with ECG-Monitoring. ECG monitor shows inferior wall STEMI and patients vitals. The EKG, 12-lead EKG and vitals automatically upload into the pre-hospital care report. In the chief complaint section, the appropriate term follows “suspected STEMI inferior wall MI” the vitals and respected vital section is updated automatically.

The 12-lead and real time mirror image of the ECG monitor is sent to the ER, for proper triage, the Cath Lab for the cardiologist to see live, and confirm if it is a STEMI, and start planning ahead for where the blockage is located and proper treatment, and imports the appropriate “data” into the triage nurses computer system as well, instead of needing to manually enter in the entire verbal report from EMS. After the PCR is completed HIPPA compliant demographics, 65 year old male Inferior Wall MI, with attached hx,medications, final treatment, type of stent, medication coating, etc. is compared with future admissions, MI's, etc. and is sorted and sent to a library to be part of the Epidemiology section (which includes the data visualization library) and used for statistics, and research.

Patient name address, health insurance information and other HIPAA non-compliant data is not collectedly the present embodiments or the System Plug but is ‘read’ by the system. The present embodiments remain complaint with the US Health Insurance Portability and Accountability Act (HIPPA), EU General Data Protection Regulation (GDPR), and Swiss Federal Act on Data Protection (FADP).

Current methods. Currently ECG uploads are manually or via Bluetooth, Wi-Fi or via cable. This is a separate system to be able to transmit the 12-lead to a hospital. Limitations to 12-lead transmission are limited to cost, of purchasing certain hardware making the monitor compatible to be transmitted to the hospitals current system. Often Paramedics need to use their phone or other non HIPAA compliant methods of communication to send pictures of the 12-lead to the Emergency Physicians phone for consult, or early STEMI recognition and activation in the ER.

Improvements. The present embodiments connect to hardware and software in and out of the “part of the system family due to its ability to read the API, learn its syntax and automatically connect it to the “System Plugs API”. Then the System Plug takes and sorts the information into the other modules and components as needed for their specific use case. The System Plug also sorts and stores appropriate data to be used to optimize the system in real time—ranging from decreasing response times and location in EMS, hospital preparation, and cuts down provider time by decreasing onset-to-balloon time by streamlining communication and early onset notification to the Cath lab team.

Example 2. Use-Case of the System Plug Ultra sonography in the pre-hospital environment. Consider the case of identifying a Deep Vein Thrombosis, the USG is automatically transmitted in mirror image via appropriate telemedicine physician for observation and attached to the report automatically including thrombus size, location, and differential. The same concept of automating the “data” is applied to the appropriate user's documentation report. The chief complaint is automated as the diagnosis, “DVT”, in assessment, the assessment findings auto populate.

This also helps expand the role of the paramedic, if used in a community paramedic program, this patient can be treated on scene with treatment of blood thinners, either IV, subQ or PO as prescribed by the physician. If in the case of a community paramedic program, the assessment findings are also auto populated in the Paramedics PCR as well. System Plug sorts and package the information to its appropriate location, including a library to be part of the Epidemiology section and used for statistics, research, and tracking.

Current methods. Currently if a patient has leg pain, and their physician is concerned for the possibility of DVT. The physician most of the time tells the patient they need to go to the hospital. Concerned for the risk of a DVT means the patient is at risk of a pulmonary embolism, which can lead to death. The second common scenario is the patient calls 911 scared they have leg pain, and they don't know what it is, so they call 911. The use of USG is not a regular practice in paramedicine but has other uses-cases in telemedicine, and home visiting services which utilize USG technicians. A USG technician reports his assessment findings to the physician and then a decision for patient treatment is made—not in real-time. With the advancements of butterfly networks technology to simplify the ability to make a differential diagnosis, one can develop and introduce many new programs into health care. This technology is used to expand the ability to combine the use of USG in a community paramedic program to diagnosis, and treat the patient at home, therefor cutting down the cost of treatment.

Improvements. The System Plug connects to hardware and software in and out of the “part of the System family as mentioned above. Then the System Plug takes and sorts the information into the other modules and components as needed for their specific use case. The System Plug also sorts and stores appropriate data to be used to optimize the system in real-time. Then the System Plug takes and sorts the information into the other modules and components as needed for their specific use case, either the health care providers report, mirror-imaging diagnostics for the use of telemedicine, and epidemiology section. The System also introduces the ability to introduce much needed programs, and create different use cases from current practice, which cut down the overall cost on health care. The System Plug provides the ability to synch a health care field, while provide the ability for the health care field to work in synchrony.

Example 3. Future Use-Case of the system Plug with PVID. PVID (see diagnostic equipment) has multiple use cases in the identification and treatment of stroke and MI. Presented here is the use of early stroke recognition and early administration of t-PA. In this example, PVID identified that the patient has a thrombus in the middle cerebral artery and is having an ischemic stroke. The diagnosis, size and position of clot is automatically sent to the System Plug to be distributed to the paramedics report pre-hospital care report (PCR), as stated in the previous examples. The System Plug simultaneously mirror images the program to the neurologist at the hospital, or if the neurologist is on-call and not yet at the hospital, they can still see the diagnostic image on his device through the present embodiments interface and can communicate directly with the crew on scene with the patient, and the emergency department of the receiving hospital. The neurologist may order the standard dose of t-PA for the given patient, and the crew on scene can start to administer t-PA if in the safe window of opportunity.

The System Plug sorts the information and places the appropriate data in each providers documentation service, along with the appropriate data in the library to be part of the Epidemiology section and used for statistics, research, and tracking, as in all other use cases. The System Plug makes this a 3 step process: The patient calls 911 4 EMS definitively diagnoses the stroke with PVID 4 neurologist simultaneously sees the image and determines appropriate treatment.

Current methods. Currently all methods of pre-hospital diagnosis of stroke for early recognition of stroke and implementation of t-PA treatment, are implemented in pilot programs in dense populations with the need of a team of specialists to respond in an ambulance with a mobile CT-Machine. Other limitations include cost, and location of implementation which is urban settings and healthcare systems which have the financial freedom and ability to implement such programs. Information is shared via phone and communicated through multiple different platforms and parts. Most common stroke activation to t-PA looks like this.

Currently this is a 7-step process: Patient calls 911 4 EMS recognizes patient is having a stroke 4 EMS notifies the receiving facility 4 the receiving facility activated a stroke response 4 the neurologist and team then goes to the ER to meet the patient 4 the patient is then rushed into CT 4 and then appropriate treatment is determined. Improvements here are that the System Plug makes possible for the automation of synchronizing such a process in one system with the added benefit of automatically entering the diagnostic information, in the documentation for the provider. In this example, the current method is a 7-step process. The System Plug simplifies a 7 step process to a 3 step process along with the other added benefits described in the previous examples.

ER triaging. In the event of COVID-19, it became known to the public eye that hospitals have been becoming overwhelmed with patients. Due to the rise in the number of patients, emergency calls, and decrease of available staff. Emergency departments are often overwhelmed with the sudden arrival of many critical and non-critical patients at once. Already described above is how the System Plug sorts of the information to the appropriate place including mirror imaging the emergency department with the ECG monitor. However, this can go one step further, and the patient condition can be triaged in the appropriate color triage according to the hospitals own internal policy. Parameters can be changed by each individual hospitals policies, and always overridden by the provider.

Currently a system sold under the name TWIAGE, is a mobile app adopted used by some hospitals to simplify the triage process which requires manually entering data into the app and sending it to the hospital so the hospital then triage the patient. In some EMS systems phone reports or radio reports are given to the ER and the user manually triages the patient. In other EMS systems hospitals do not get any report from EMS unless the patient is critical. In the event of an MCI hospitals are notified by the provider or incident command when the incident occurs.

Triage is a human performed skill, either in EMS or in-hospital. The System Plug mirrors patient information and diagnostics in real time to assist with and streamline the triage assessment simultaneously while also providing the EMS provider with the information if the hospital is at full capacity in real time. The present embodiment predictive algorithms also act as a tool for emergency rooms to prepare in advance for mass casualty incidences so first responders and emergency departments may plan and prepare for these unexpected and demanding events.

In-hospital diagnostics. The use of in-hospital vital machines, USG, CT, MRI, etc are not connected. When a patient enters into a hospital and feel sick, they go through triage in the emergency department. The nurse takes vitals, and then enters them into the documentation system, the patient then goes to the appropriate triage area, further diagnostics are performed, and then manually entered the patient's chart, etc. The Present Embodiments connect all these devices to automatically enter the appropriate information in each section of the chart for each patient in the entire hospital. Information is sent from the device to the System Plug then to the chart. Following the concept and examples from devices in pre-hospital devices. Instead of sending CD's with patients of records of diagnostics, information is mirrored in real-time and sent via the System Plug, simplifying the patient sharing process.

Under current practice, when diagnostics are made, they are manually entered into the charting system. In telemedicine physician consult is performed via screen and camera, requiring such device to be present and program in implementation. Medical records are shared via CD's, faxed, in verbal report, etc.

The present embodiments provide the ability to mirror and share the data in real time, while connecting all the diagnostic equipment in and out of a hospital regardless of manufacturer and health care system, but allows it to remain separate as well, keeping the infrastructure secured. Diagnostic information automatically gets updated in the patients' charts simplifying and automating the process of manually entering the data.

Telemedicine is a growing field with multiple benefits, in decreasing the burden on the health care system for providers and decreasing overall costs of health care. The present system expands the use-cases for telemedicine, by simultaneously mirroring diagnostics, and creating a streamline method of communication between provider and physician in one system, while automating documentation.

For example, telemedicine today has a multitude implementations. One example is in the ER in the triage dept. There is a Physician consult in a screen to assist the triage nurse in priority of patient status if there is question. Telemedicine is also used for medical translation in-hospital. There are mobile containers placed as telemedicine consulting areas. However, each use case is compartmentalized and limited by its use to single use cases in single locations.

The present embodiments expand the use of telemedicine. It also centralizes a decentralized system while automating documentation, and taking information for research purposes, to further optimize. The present embodiments' predictions make for more accurate results in real time and for other researchers, scientists, doctors, and epidemiologists to pull the data and conduct their own research from the systems library.

The present embodiments automaticity opens the door for expanding the use for community paramedicine, and other home service programs to provide lifesaving treatment at the patient's side, decrease hospital stays, and lower hospital admittance, therefore decreasing cost. Research supports that even cutting down door-to-balloon time by 1 minute can save 2% heart sufficiency. Therefore, early recognition and diagnosis of acute myocardial infarction as well as and treatment of patients at home become essential in a prehospital environment. The same can be said for patience experiencing a stroke.

The demand for proper documentation increases with more requirements from Medicare/Medicaid and insurances trying to find way out of reimbursing for medical treatment, procedures and interfacility transfers. With the increasing amount of technology used in health care, this also leads to more screen time from health care practitioners instead of patients. With increase demand of patients, and increased need for proper documentation, there becomes an increase of stress with health care practitioners and increase in possible documentation errors. Hospitals have recognized the increased burden on physicians years ago, from implementing a position called a scribe, whom is a supplemental employee who assists the physician in their assessment. Other advancements have been introduced such as speech to text devices stationary at the physician's desk to increase time for documentation completion. In many situations many different healthcare practitioners document the same thing but for their own purposes. Refer to the following example:

For example, a patient calls 911 for a stroke. EMS finds the 71-year-old male patient to have right sided facial droop and left sided arm weakness. The patient is hypertensive, blood glucose is 134 mg/dL. EMS activates a stroke response at the local ER. The RN at the ER needs to document her own report, the Emergency Physician needs to document his own report, and the Neurologist who meets the patient in the ER needs to document his own report. Through this process providers pass on a report, list of medications, hx and the like. Some of which are in their system some of which are not, and have to manually enter the diagnostics which are performed for example, vitals, EKG, CT, etc.

It would be inappropriate to completely link documents, due to individual provider errors, and patient changes. However, there are parts of the story which can be automated. Already discussed are how the diagnostics can be automated into the documentation. Now let's discuss the ‘patient-hand offs’. In each stage, the previous provider gives report to the new provider, then the new provider documents, the report given, this can be auto populated from chart A→chart B. Also, the chief complaint the triage nurse writes down is always given by the ambulance coming in the hospital, this can be automated, along with medications, hx, etc. However sometimes, the hospital, has more up to date accurate information than the ambulance crew, this can be two ways to be updated into the EMS crews PCR. When an EMS crew goes to the hospital, the patient needs to be registered into their system. This is done autonomously prior to arrival by linking the PCR to the hospitals registration system through the System Plug by sending the required information. All components are separated by the System Plug, sorted, and packed by the System Plug and sent to the appropriate location to create a more efficient work environment.

Currently Patient reports are given verbally, and providers manually enter in the information provided from the report, every provider enters their own assessment, medical records, hx, etc. Every provider needs to enter their own diagnostics even if it is for a shared patient.

The present embodiments automates entering patient reports from patient hand offs, cut down time within one's own time spent documenting, by automating the process, automatically enter the diagnostic information without having to manually import it, and fill out the document in the appropriate sections. This cuts down on-screen time, streamlines workflow, and minimizes documentation errors.

AI-assisted scribe. Medical scribes are a newly instituted solution over the past decade to assist physicians with the overwhelming amount of documenting they are required to do, and the increased in the number of patients they are meeting on a daily basis. With the development of speech to text AI technology and recognition, this process is automated. With a tiny microphone attachment pinned to the physicians, paramedics, or nurse's shirt, the entire assessment is recorded and transcribed into the documentation in the appropriate format. For example, the chief complaint, is automatically entered, in the appropriate section, the patient complaints are listed in the narrative, the patient assessment is automated and entered into the narrative. Again, the document is linked to the present system and appropriate information is sorted and sent to streamline information as needed including always the epidemiology section.

Currently hospitals pay for scribes only for physicians and upper levels. This cost money and still always leaves the room for provider error. And all information is entered and typed manually.

The AI-assisted scribe of the present embodiments automatically recognizes the providers voice with the opposing voices, take the answers and sort them as complaints, medical hx, assessment, name, demographics, medications, etc. Automatically entering this information in the appropriate section of the chart including the narrative section. This saves time on a process which takes time away from patients and increases the time a provider has talking to each patient and minimizing documentation errors.

Overall, combining the use of NLP Natural Language Processing and deep learning, the present embodiments integrates with devices outside of the system family, automating the process of API connection. This eliminates the step of someone needing to manually connect the API of two programs and calling the company on program A receiving their API and having coder from program B to connect it. This also eliminates the problem of having two programs not compatible in the case of incompatibility. This streamlines communications with providers in many different roles to increase the ability of providing definitive care into patient's homes, keeping patients out of hospitals, and treating patients at home when appropriate. This can help decrease the amount of nosocomial infections and decrease the overall cost of health care. The present systems simultaneously allows this information to become available to be processed for research purposes for epidemiologist to pull information and play with the data in the epidemiology section to predict future epidemics, supply usage from the documentation is sorted into the supply and resource management section to predict future supply usage and manufacture (referring to OSM) or order the appropriate number of supplies. All the modules, even the ones not mentioned are connected and automatically updated by the information being processed and sorted through the System Plug along with automating documentation simplifying and maximizing a multiple step process with multiple added benefits including a quicker real time way to reprocess data and retrain the algorithms for more accurate predictions.

The Present Systems also opens the door for more advanced in-hospital treatments which are not yet performed in the pre-hospital setting to be performed in the pre-hospital setting, for example heart catheterizations, invasive and non-invasive stroke treatment, etc. Opening the door for more expansive treatment in the pre-hospital environment and forecasting data, opens the door to introduce new policies, programs, and standards in care. For example, forecasting department patient admittance stays, facilities may cross train staff in the event to utilize staff more appropriately and introduce new internal policies and educational tools. Hospitals can specialize certain mid-levels for casting, suturing, intubating, leading cardiac arrests, etc., to relieve the burden on physicians and upper levels to perform these tasks. The number of possibilities of how this tool can be used are endless specific to each region, health care system etc. The System Plug is used as a tool, acting as a Lego board for the Lego set to provide each healthcare system with the information, they need to determine how to use it for what is best for them. The System Plug is the integration center of the present embodiments and allows the system to operate in a way where it becomes centralized, centralizing a decentralized health care system. Eliminating many problems such as limited access to information, and patients not having access to their own records, having troubles and delays in sharing important health care information when not possible and more.

Automated user interface (UI)-based on type of learner. The Present System implements the ability for automated UI design based on the natural eye movement of the specified user. People often don't pay attention to how they read, learn, and absorb information. There has recently been a push for the need for specifically designed textbooks that are adaptive for each type of learner. There is upcoming research trying to pinpoint saccadic eye movement towards automatically generating a UI design based on users' specific saccadic eye movements. The need for a UI design that provides health care professionals to glance at the screen and intuitively use the software becomes crucial in implementing new healthcare software. Also, because technology and screens have become an integral part of the healthcare field, interference is introduced; this requires implementing a UI design that does not interfere with the provider's workflow.

The Present Systems' UI is designed not to interfere with the provider's daily workload, incorporate all the information necessary on one page, which is absorbed in a glance. The automated UI system adapts to the natural way the user absorbs information, developing a series of 3 or 4 images, which are selected based on the user's natural saccadic eye movement. This system automatically generates an intuitive user experience that conforms with the subconscious cues of the healthcare professional.

The technology and understanding of developing an algorithm for automated generated UI are in their infancy. There is not a large enough push for the need within the culture to create an algorithm and large implementation. Attempts to develop such an algorithm have led to the conclusion that the difference between applying basic UI rules—maximizing contrast, minimizing information on a page, large font size, certain font types, and proper spacing is almost as good as an algorithm which creates a unique UI based on the users saccadic eye movement. The algorithm produced UI designs, compared to existing UI designs utilizing the core concepts, did not resemble much of a difference.

Diagnostic equipment.

The development of medical equipment today lacks a strong interworking relationship with field providers and the people designing the equipment. It lacks the understanding of developing certain tools which are beneficial for certain health care systems and environments but not others. The role of the paramedic has grown drastically over the last 50 years and being able to provide tools and instruments to further expand the role that emergency medical services may play has countless benefits. Myocardial infraction is the number one reason of death in the world and fighting it requires the extension advance diagnostic tools and invasive procedures in the pre-hospital environment. Not only does this lead to a tremendous increase of quality of patient care, but also can lead to the development of more efficient methods for in-hospital treatment.

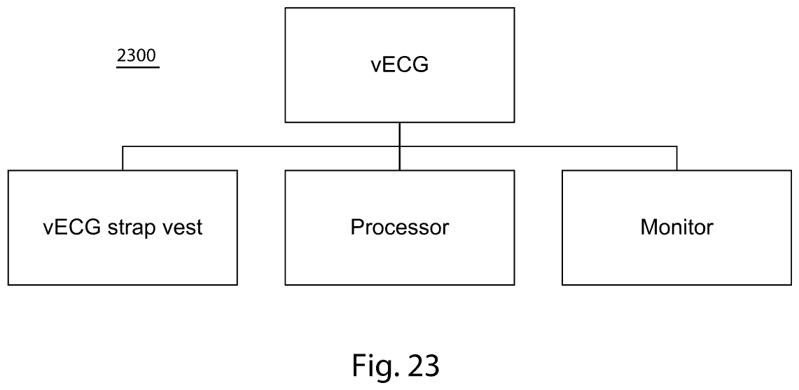

The vECG. See FIG. 23

One solution is an all vector ECG. The all-vector electrocardiograph (vECG) is a diagnostic imaging device which provides a three-dimensional image of the heart based on the electric impulses generated by the heart. Providers use regular 12 lead ECGs to assess function and conductivity of the heart in order to find pathological disturbances with astonishing accuracy. However, learning to interpret a 12 lead ECG takes months of training, and years of experience to become a true expert. The vECG takes a huge part of the learning curve away, by providing a visual representation of the heart in real time. This provides huge benefits across all medical fields, of instance field-providers needing to diagnose patients in emergency situation or cardiologist who benefit from the extended diagnostic capabilities and patient education compared to regular ECG.

The vECG is also an integral component of current systems' PVID which has the capability supporting interventional treatment along with diagnostic capabilities for stroke and myocardial infarction in the pre-hospital environment (see below). It is a system which uses broad-captured ultrasonography and enhanced computerized graphics based on the present systems vECG monitoring capabilities to provide a clear image of affect heart structures and vessels to perform stent catheterization.

vECG strap vest: It has of a set of electrodes which are incorporated in a stretchy fabric similar to the material used inleggings, allowing for the best chest coverage regardless of chest circumference or gender. Unlike a regular vest, the strap vest is unfolded to a sheet so that it may be put on patients in supine position. An indicator line at the front of the vest serves as an orientation mark for midsternal alinement.

Processor: The processor receives the analogue date from the electrodes and converts it to digital information. Depending on the strength and difference of the receiving signal between concordant electrodes vectors are calculated. The origin of the signal is then traced using triangulation of the established vectors.

Software: The software calculates area of heart or projects information received from the signal onto prerendered 3D model to show heart depolarization over its surface. To prevent error the image cleaned and amplified, if necessary.

Monitor: The monitor shows a live three-dimensional rendering of the heart. Anatomical structure such as atria, and ventricles are represented in even colors whereas pathologies (or in other word lack of signals) is highlighted in signaling colors. Each full depolarization-repolarization cycle is viewed individually, like the individual picture of a video, or is fused together to create a full structure. The monitor has a touch screen to allow provider to rotate the image and highlight abnormalities or areas of interest.

Procedure. The vECG strap vest is placed on the patient's chest. Depending on the patients position or mobility the vest can be unfolded to assist the patient with the placement or in case the patient is unconscious the providers are able to wrap the vest around the patient's chest by themselves. The provider proves the alignment of the vest to the midsternal line and tightens the vest to the patient's body. In the next step the monitor is turned on. A window reminds the provider of all the above-mentioned steps. The system starts processing the signals. Just like on a regular ECG the system needs a few seconds before a full image is generated. Providers observe live depolarization-repolarization cycles of the heart in order to start their analysis.

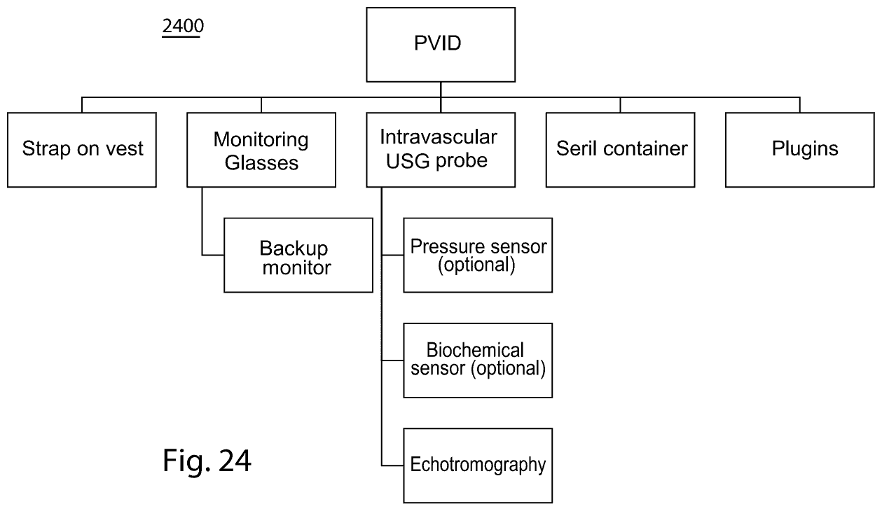

### PVID. See FIG. 24

The Present System's Portable Vascular Imaging Device (PVID) is an ultrasonography based portable imaging device which has the capability of supporting interventional treatment along with diagnostic capabilities for stroke and myocardial infarction in the pre-hospital environment. Ambulance and helicopter crews carry this device and use it as an instrument for diagnosis and treatment at the side of incidence. This device provides the capability of pre-hospital cardiac stent catheterizations as well as neurological stent catheterizations in future iterations, if necessary.

This device not only provides an extension for the treatment of patients suffering from stroke and myocardial infarction, improving door-to-balloon time and survivability rates, but also open the door to other future surgical interventions performed in the pre-hospital environment, increasing quality of acute patient care. Creating a more cost-effective substitution for current treatment provided out-of-hospital could paves the way to augment or even replace current in-hospital treatment methods, for example cardiac catheterization labs and interventional radiology.

Medical innovation is moving towards creating smaller and more portable devices. However, at this stage the innovation limits the idea of extending care outside of the hospital environment and is focused on using classical imaging technologies such as CT and MRI. The Present System uses broad spectrum ultrasonography and enhanced computerized graphics based on the present systems vECG imaging device to provide a clear image of affect heart structures.

In addition, the standard kid contains a carrying matte with legs, sterile gloves, masks and operation coats. The catheters are in a sterile container which is already prepped and hooked up to the main computer. The system is battery powered with four hours running time and it includes a power cord with the standard electrical output for the given nation.

The system is configured for 2 people to perform the intervention.

Strap on vest. Based on the vECG vest, which monitors the vector shifts of the electrical condition system of the heart, the PVID strap on vest acts as a receiver (optional) for the intravascular USG probe. Using the imaging modalities of both USG and vECG a 3D image of the heart is created.

Monitoring Glasses. The present system comes with 2 to 3 mixed reality monitoring glasses which are uses the display the heart. The glasses are activated as soon as they are put on and set up automatically. The heart is displayed as a 3D image/rendering above the patient's chest in the mixed reality space. Various other windows are open upon the mixed reality space, hovering and surrounding the 3D image of the heart. Displayed data includes, but is not limited to:
ECG
Heart rate
O2 saturation
vECG Image (3D model)
Ultrasound (transesophageal, intravascular)
Biochemical data
   The device is setup for personal preferences. The advantage lies in its portability and requires no installation of chunky screens. Moreover, hands are liberated for work and kept clean as the operator of the device does not interact with a physical screen. However, for redundancy purposes a 15-inch flatscreen is included to the system.

Intravascular ultrasonography (USG) probe or Multifunctional catheter. This device has a long tube, which is inserted through the femoral of radial artery to access the stem of the aorta and is used to establish echotomography of the heart. The sensor must have a diameter not greater than 5 mm to be inserted into the ascending aorta via the radial or femoral artery. The Ultrasound sensor is located at the tip, which is angled to point outwards and can be rotated in its Y-axis. Distally form the USG prove a balloon is located onto which the stent is mounted.

Sterile container. Includes roll-out catheters for guidance and stent equipment, to mount the stent onto the Intravascular USG probe.

Plugins. Various other equipment is provided onsite e.g. an oximeter to check the pulse wave and O2 saturation of the patient.

Software. The software adds the images form the ultrasound and vECG together, creating and overlaying image that shows a real-time 3D rendering of the heart activity. To amplify small structures such as vessels the image is processed by tracing the structures via the electrical activity over the surface of the heart. This allows to fill gaps in the image, if necessary, and illuminating structures that are not easily visualized via ultrasound such as the inferior border of the heart.

Procedure. For preparation, the vECG vest is placed on the patient's chest who is then transferred onto the carrying matte which is adjusted to the high of the operator. Both Operator put on the Monitoring glasses. Operator 2 sterilizes the hands and puts on the OR coat whereas Operator 1 assess the heart area of interest via the 3D image and the severity of vessel occlusion. Airways and pulse oximetry are assessed and IV lines are started by operator 2. Morphine is administered to the patient (administration of medication is determined by the current Cath lab guidelines). Operator 1 assesses intravascular access. The areas of insertion are then sterilized and covered with sterile cloths. Operator 1 mounts the intravascular ultrasound and operator 2 prepares the roll out catheter with guiding wire and stenting equipment. Depending on the severity of vessel blockage, the appropriate stent length is added to the probe.

Execution. The operation begins: Operator 1 inserts the intravascular ultrasound via the femoral or radial artery. Ones the ascending aorta is reached, the image is reassessed. Operator one holds the probe in position while operator two inserts the guiding wire via a side opening of the intravascular USG. The wire is push forward into the affected vessel to provide guidance for the USG prove.

Option: The roll out catheter may include a pressure sensor and or a biochemical sensor, if feasible, in future iterations. These are used to measure the pressure difference inside the arteries in the cardiac cycle, which may give further insides in the degree of vessel occlusion as well as be of value in assessing if the stent placement was successful.

As soon as the guide wire reaches its target, the probe is pushed forward to the occlude vessel and the stent is placed via a balloon mechanism, which pushed the stent in place. Operator two pulls the probe back and the vessel is reassessed.

Option: Pressure and/or biochemical measurements, if feasible, are taken to evaluate the stent placement. If successful, the intravascular catheter is pulled out and the wound is closed. After the procedure the patient remains on stretcher and is transferred to the nearest medical facility for monitoring.

Education. Health care currently focuses training to simplify the education process to achieve the largest usable result from a single provider. For example, a physician the highest level of education, and training creates a health care provider who has more understanding of a subject and can react accordingly when met with a difficult cases. A paramedic is uneducated with a minimal level of education, specified in emergency medicine. They are specially trained in advanced adult and pediatric cardiovascular care. They can perform surgical cricothyrotomies, transcutaneous pacing, synchronize cardioversion, run an entire CPR and trained to interpret EKGs, and perform physician-level skills. However, if a Paramedic is asked, what the term histology means, they probably are unaware it is even a subject, yet alone the study of tissue. This is an example of how specialized and focused education becomes extremely important in performing a number of tasks. The more understanding of a subject, the more diverse and complex tasks a provider can perform. This is not a new concept, but one which is adopted in the health care system today separating Nurses, Paramedics, Physicians, Specialists, lab technicians, pharmacists, respiratory therapists, etc.

Isolated skills can be taught to anyone without a deep level of understanding. In fact, many paramedics utilize civilians in extreme situations, and teach them how to assist in ventilations or do chest compressions during a CPR if necessary. With the increase demand of health care professionals, and rise of patient admittances, evaluations and 911 calls. The need for expanding the health care system is growing.

Mass Casualty Incidents are not limited to specific traumatic incidents or terrorism, or chemical warfare, or a tour bus rolling over, but have been observed by sudden spikes in illness such as the COVID-19 pandemic. It is becoming more and more evident for the proper need of resource utilization, and staff utilization.

The importance of switching roles, and cross training is an old concept utilized by the military, and space agencies such as NASA and ESA. Space health and disaster medicine can learn from one another. The training of astronauts to work in challenging conditions has produced relevant knowledge for the training of a sustainable pool of professionals ready to deploy to disaster settings.

Several authors have discussed the skills required of medical teams in response to disaster, or teams deployed on space missions in isolation from one another. Others have discussed space training programs with specific regard to their applicability to remote and disaster medicine, such as the European Space Agency's CAVES training program. Similarly, the need for international cooperation and teamwork, identified in the literature regarding disaster responses, mirrors some key qualities required of astronauts working in international teams to perform highly skilled tasks under significant pressure, as identified from the earliest Mercury mission training. Though difficulties exist in both disaster and space settings, it is possible for each field to learn from the other with regard to the training of mission staff.

This section serves as a supportive section to the present embodiments to demonstrate more how the present embodiments are a tool for all health care settings and environments to operate at maximal capacity with the minimal amount of resources.

The present embodiments are developed to be a tool for every health care system. This allows every individual EMS agency, Hospital, and health care system to use the present embodiments in the way it fits for them so they may optimize their own operations and logistics and help prepare and plan for future events.

With machine learning based forecasting, health care systems can prepare in advance for MCI's and plan proper trainings around suspected MCI's. Other trainings can be focused based on short term suspected caseloads predicted by the present embodiments.

Cross training providers can be chosen more intelligently by determining if one wants to cross train providers from slow departments for the busiest, or cross train providers for solely the busiest departments in closely related fields.

The present embodiments use easy user interface and recognize the importance of minimizing screen time for health care professionals. Clinical studies demonstrate certain focal points fora UI design for learning disabled, or dyslexics. Other advancements are made to create AI-assisted UI design to adjust to the user's saccadic eye movement. The field of data visualization was developed to process and interpret large amounts of information in a simple way, one rule of thumb in data visualization is to minimize the amount of information on one page for the user to not be overloaded. The present embodiments incorporates a research-based UI design and methodology behind all of the system's UI design.

However, the present embodiments would like to take this one step further. The development of educational materials represented in different methods to allow to educate a provider with the most understanding in the least amount of time, either for cross training, or just working in the front line, how the provider is used is up to the health care system, institution or agency.

By developing more expansive educative materials and introducing a tool to help institutions focus their training, planning and preparation, an institution can implement new policies, procedures, and methods in how to respond to day-to-day practice and Mass Casualty Incidents. Combined with present system devices such as the vECG or PVID, the expansion of who can perform such advanced and invasive treatment allows a new style of medicine to enter into the field. Allowing patients to be treated sooner, helps save lives, decreases the burden on the entire health care industry and lowers the overall cost of health care expenditure. An illustrated child textbook, can help a provider understand a story behind a process, and a disease better while allowing them to quickly review as needed to keep the information fresh. 3D animated videos showing concepts instead of the current videos which are computerized lectures with pictures, can allow a provider to better understand a see a physiological process, pathomechansim or biochemical process. This allows the ability to combine, subjects in one sitting. For example, to see a physiological process, with the biochemical process together becomes simpler to understand than having to put it together oneself (which here becomes the separation of IQ and ability in the academic world, who can put information together themselves, or simply wants to, or understands how to) This allows to expand the ability for providers of different capabilities and academic backgrounds to grasp material and expand their ability to impact the health care system they work in.

The self-sustainable hospital. The present embodiments are designed to be the smart infrastructure for the self-sustaining hospital concept and backbone of a sustainable health care ecosystem. The healthcare industry is facing many problems, form inefficiency, and self-destructive behaviors to implementation problems. The root problems in health care can be summarized as improper staff utilization (staff shortages), supply shortages, and broken communication. There are many branching problems from these rooted problems, for example staff shortages, high patient to staff ratios, delays in patient care, increased in hospital staff burnout, More staff quitting, back to staff shortages, delays in care also lead to increased patient admittance stays, total increased health care expenditure.

By driving medical procedures and care into the pre-hospital environment, it forces the medical device to be cheaper, portable, and safer. By treating patients sooner, one can decrease hospital admittance stays, or completely remove the need for hospitalization entirely. This allows for an overall decrease in health care expenditure. By decreasing patient admittance duration, and hospital admittances, one can lower patient to staff ratios, this has added benefits to both allowing hospital staff to spend more time with different patients and decrease provider burnout. Interestingly enough, if one can develop portable medical devices with multiple functionalities, it eventually replaces the procedures and devices used in a hospital.

Society has already driven the advancement of sustainable energy, let's take the concept of sustainability one step further. By removing medical waste, and introducing new methods, and technologies to allow for the manufacturing of analog and non-analog medical devices, and medications on site of a facility—the concept of a self-sustaining hospital becomes possible.

The development of 3D printing foundations, and modular buildings has flooded the industrial market, cutting down the cost and time to build a house or industrial building. 3D printing foundations is a new concept, still trying to find its niche and introduction to the market on a large scale.

The present systems seek to lay the foundation to introduce a fully self-sustainable hospital, equipped with the necessary needs to operate, and produce medical consumables on-site. A fully self-sustained hospital system can be built anywhere in the world and has the ability to function at maximal capacity in any times of any crises, either, economical, ecological, or epidemiological. Experts from a variety of fields which are normally not represented in the health care field, need to be present and part of such a project. Bridging industries, and industry languages becomes vital in making a self-sustaining hospital a reality.

Again, the present embodiments are involved in the constructive process of a new facility, or help administrators restructure existing facilities. By forecasting epidemiological mapping, with the present embodiments' machine learning algorithms determine the most optimal location where hospitals and medical facilities should be established. The most optimal size units based on bed and capabilities for both existing and newly constructed hospitals is determined. As facilities face unexpected new challenges, and currently downsize departments, and other departments seem to be flooded and overwhelmed with patients. By using the present embodiments' predictive algorithms hospitals may continuously determine the need for department size, number of beds, supplies, and capabilities needed in advance. With the continuous flow of new patients, means new data, and in real time makes new more accurate predictions.

The present embodiments are designed to be the smart infrastructure for the self-sustaining hospital concept and backbone of a sustainable health care ecosystem. The present embodiments are designed to be the Lego board of the Lego set in the health care field. With many new advancements in separate fields of health technologies, the present embodiments is created to be the central link of all present and future advancements. Allowing separate industries, and technologies to operate in a harmonic way, just as an orchestra is led by its conductor to combine wind, brass, and string instruments, to create a beautiful symphony, The resent embodiments aim to bridge all aspects in health care to allow the health care system to operate in harmony.

A network data processing system can be a set of networked computers in which the present embodiments may be implemented. A network data processing system can connect to a network, which is the medium used to provide communications links between an instance of the present embodiments and various computer servers and devices. The network may include physical connections, such as wire, wireless communication links, or fiber optic cables and may be an intranet, wide area network or the Internet. Networked servers and devices can collect and process and send processed data acquired from networked devices.

A network data processing system may include additional servers, clients, and other devices not shown. The network of the network data processing system can be an Internet network representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, having thousands of commercial, government, educational and other computer systems that route data and messages. The network data processing system also may be implemented as a number, one or more, of different types of networks, such as for example, an intranet, a local area network (LAN), or a wide area network (WAN).

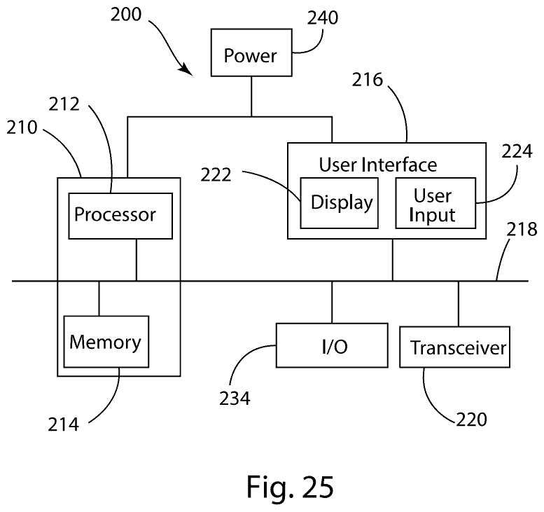

Further, the processes, methods, techniques, circuitry, systems, devices, functionality, services, servers, sources and the like described herein may be utilized, implemented and/or run on many different types of devices and/or systems. Referring to FIG. 25, there is illustrated an exemplary system 200 that may be used for many such implementations, in accordance with some embodiments. One or more components of the system 200 may be used for implementing any circuitry, system, functionality, apparatus or device mentioned above or below, or parts of such circuitry, functionality, systems, apparatuses or devices, such as for example any of the above or below mentioned computing device, the systems and methods of the present embodiments, request processing functionality, monitoring functionality, analysis functionality, additionally evaluation functionality and/or other such circuitry, functionality and/or devices. However, the use of the system 200 or any portion thereof is certainly not required.

By way of example, the system 200 may comprise a controller or processor module, memory 214, and one or more communication links, paths, buses or the like 218. Some embodiments may include a user interface 216, and/or a power source or supply 240. The controller 212 can be implemented through one or more processors, microprocessors, central processing unit, logic, local digital storage, firmware, software, and/or other control hardware and/or software, and may be used to execute or assist in executing the steps of the processes, methods, functionality and techniques described herein, and control various communications, programs, content, listings, services, interfaces, logging, reporting, etc. Further, in some embodiments, the controller 212 can be part of control circuitry and/or a control system 210, which may be implemented through one or more processors with access to one or more memory 214. The user interface 216 can allow a user to interact with the system 200 and receive information through the system. In some instances, the user interface 216 includes a display 222 and/or one or more user inputs 224, such as a button, touch screen, track ball, keyboard, mouse, etc., which can be part of or wired or wirelessly coupled with the system 200.

Typically, the system 200 further includes one or more communication interfaces, ports, transceivers 220 and the like allowing the system 200 to communication over a communication bus, a distributed network, a local network, the Internet, communication link 218, other networks or communication channels with other devices and/or other such communications or combinations thereof. Further the transceiver 220 can be configured for wired, wireless, optical, fiber optical cable or other such communication configurations or combinations of such communications. Some embodiments include one or more input/output (I/O) (Inport/Outport) ports 234 that allow one or more devices to couple with the system 200. The I/O (Inport/Outport) ports can be substantially any relevant port or combinations of ports, such as but not limited to USB (Universal Serial Bus), Ethernet, or other such ports.

The system 200 comprises an example of a control and/or processor-based system with the controller 212. Again, the controller 212 can be implemented through one or more processors, controllers, central processing units, logic, software and the like. Further, in some implementations the controller 212 may provide multiprocessor functionality.

The memory 214, which can be accessed by the controller 212, typically includes one or more processor readable and/or computer readable media accessed by at least the controller 212, and can include volatile and/or nonvolatile media, such as RAM (Random Access Memory), ROM (Read Only Memory), EEPROM (Electrically Erasable Programmable Read-only Memory), flash memory and/or other memory technology. Further, the memory 214 is shown as internal to the system 210; however, the memory 214 can be internal, external or a combination of internal and external memory. Similarly, some or all of the memory 214 can be internal, external or a combination of internal and external memory of the controller 212. The external memory can be substantially any relevant memory such as, but not limited to, one or more of flash memory secure digital (SD) card, universal serial bus (USB) stick or drive, other memory cards, hard drive and other such memory or combinations of such memory. The memory 214 can store code, software, executables, scripts, data, content, lists, programming, programs, log or history data, user information and the like.

Some of the present embodiments may be installed on the computing device that receives data transaction requests from the computing device from an interface. The present embodiments can be configured to process data transaction requests received through the interface. Typically, the present embodiments can be communicatively connected to a communication network (e.g., a WAN, LAN, the Internet, etc.), and has the capability of completing the data transaction requests. The present embodiments can communicationally connect with one or more remote servers that are configured to provide information useful in determining the nature of one or more data transaction requests. The present embodiments can further, in some instances, complete a data transaction request through the interface.

Further, in some applications, the remote server is implemented through and/or includes a server cluster containing multiple servers that cooperatively operate and/or communicate to provide analysis functionality. In other instances, the remote server may be implemented in part or fully on personal computer.

The present embodiments may further block access to the network access activity when the network access activity is considered an objectionable or non-compliant activity.

Third party recipients can access one or more reports in a variety of ways including, but not limited to, the report or reports being communicated by one or more of the remote servers, the third party having access to the remote server to request report, and other such methods. A request for a report can include viewing the report while the third party has access to the remote server.

In some implementations, monitoring software is installed on the computing device, and in some embodiments is part of the present embodiments. Additionally, or alternatively, some or all of the monitoring and/or monitoring program is implemented at a remote server. In some applications, the monitoring software can be voluntarily installed on the computing device by a user. In other instances, the monitoring software can be pre-installed on the computing device.

In some embodiments, network access activity can include, for example, access to one or more of the network activity from a group consisting of http, https, network news transfer protocols, file sharing programs, file transfer protocols, chat room access, peer to peer chats, game protocols, downloads of data, and electronic mail activity. The present embodiments can complete the data transaction request through the interface. In some implementations, the report can be made accessible by a third-party recipient (e.g., via direct access through a server, e-mail, periodic reports, text alerts, etc.).

One or more of the embodiments, methods, processes, approaches, and/or techniques described above or below may be implemented in one or more computer programs executable by a processor-based system. By way of example, such a processor-based system may comprise the processor-based system 200, a computer, a server, a smart phone, a smart watch, a tablet, a laptop, etc. Such a computer program may be used for executing various steps and/or features of the above or below described methods, processes and/or techniques. That is, the computer program may be adapted to cause or configure a processor-based system to execute and achieve the functions and/or functionality described above or below.

As an example, such computer programs may be used for implementing any type of tool or similar utility that uses any one or more of the above or below described embodiments, methods, processes, functionality, approaches, and/or techniques. In some embodiments, program code modules, loops, subroutines, etc., within the computer program may be used for executing various steps and/or features of the above or below described methods, processes and/or techniques. In some embodiments, the computer program may be stored or embodied on a computer readable storage or recording medium or media, such as any of the computer readable storage or recording medium or media described herein. Accordingly, some embodiments provide a processor or computer program product comprising a medium configured to embody a computer program for input to a processor or computer and a computer program embodied in the medium configured to cause the processor or computer to perform or execute steps comprising any one or more of the steps involved in any one or more of the embodiments, methods, processes, functionality, approaches, and/or techniques described herein. For example, some embodiments provide one or more computer-readable storage mediums storing one or more computer programs for use with a computer simulation, the one or more computer programs configured to cause a computer and/or processor based system to execute steps comprising: receiving data through the present embodiments that receives data transaction requests, from a local computing device on which the present embodiments are implemented, through an interface; and processing, through the present embodiments, data transaction requests received through said interface. Some cloud based embodiments further comprise completing said data transaction requests through the present embodiments that is communicatively connected via a wide area network (WAN) to a remote server which is communicatively connected to the present embodiments; wherein said remote server is configured to provide information useful in determining a nature of said data transaction request. Some embodiments additionally or alternatively comprise monitoring network access activity of the local computing device, including network activity of applications installed on said local computing device; recording results of monitoring said Internet access activity within said remote server. Additionally, some embodiments further comprise completing a data transaction request, by the present embodiments, through an interface. Further, in some instances, the Internet access activity can include access to at least one Internet activity from a group consisting of http, https, network news transfer protocols, file sharing programs, file transfer protocols, chat room access, peer to peer chats, game protocols, downloads of data, and electronic mail activity.

In some embodiments, systems, apparatuses and methods are provided herein useful to obtain product information through scanning. In some embodiments, a method performed by a circuit and/or one or more processors comprises receiving, through an interface, data transaction requests from a local computing device on which the present embodiments are implemented; processing, by the present embodiments, the data transaction requests received through said interface; and completing said data transaction requests through a communication connection with a wide area network (WAN).

Some embodiments further comprise providing information to a third-party recipient through processing functionality and/or programming of the present embodiments. Further, some embodiments comprise communicating, through the processing functionality, results of the processing to other portions of the present embodiments. Additionally, or alternatively, some embodiments comprise providing, through the processing functionality, information useful in determining a nature of the data transaction request.

Some embodiments further comprise monitoring network access activity of the local computing device through monitoring circuitry and/or functionality of the present embodiments. In some instances, the network access activity comprises network activity of applications installed on the local computing device. Further, some embodiments comprise recording results of monitoring the network access activity within the processing functionality. The network activity comprises, in some embodiments, network activity from one or more of and/or a group consisting of http, https, network news transfer protocols, file sharing programs, file transfer protocols, chat room access, peer to peer chats, game protocols, downloads of data, and electronic mail activity. Further, some embodiments comprise completing the data transaction, by the present embodiments, through the interface.

In some embodiments, one or more of the circuitry and/or functionality may be implemented external to the present embodiments and/or the present embodiments may be implemented through distinct circuitry, processors and/or functionality. For example, in some implementations, the monitoring functionality may reside on the local computing device independent from the present embodiments and be configured to send and receive data to the present embodiments. Accordingly, the spirit and scope of the present embodiments is not to be limited to the specific embodiments described.

The data processing system depicted in FIG. 12 may be, for example, a server system, running the Windows operating system, Apple OS operating system, Advanced Interactive Executive (AIX) operating system, LINUX operating system, or the like.

An operating system runs on processor and is used to coordinate and provide control of various components within data processing system. The operating system may be a commercially available operating system, such as sold under the name WINDOWS, which is available from Microsoft Corporation. An object-oriented programming system such as Java may run in conjunction with the operating system and provide calls to the operating system from Java programs or applications executing on the data processing system. “Java” is a trademark of Sun Microsystems, Inc. Instructions for the operating system, the object-oriented programming system, and applications or programs, including those of the present embodiments, are located on storage devices, such as hard disk drive, and may be loaded into main memory for execution by processor.

Those of ordinary skill in the art will appreciate that the hardware in the present embodiments may vary depending on the implementation. Other internal hardware or peripheral devices, such as flash read-only memory (ROM), equivalent nonvolatile memory, or optical disk drives and the like, may be used in addition to or in place of the hardware. Also, the processes of the present embodiments may be applied to a multiprocessor data processing system.

As a further example, data processing system may be a personal digital assistant (PDA) device or a smartphone, which is configured with ROM and/or flash ROM in order to provide nonvolatile memory for storing operating system files and/or user-generated data.

The depicted above-described example are not meant to imply architectural limitations. For example, data processing system also may be a notebook computer or hand-held computer in addition to taking the form of a PDA or a smartphone. Data processing system also may be a kiosk or a Web appliance.

Today's healthcare field faces a multitude of problems ranging from operational limitations 5100, lack of resource availably 5200 and poor distribution 5200. Emergency medical service systems in particular can often be strained 5100, which negatively effects healthcare supervision in EMS as well as hospitals 5200.

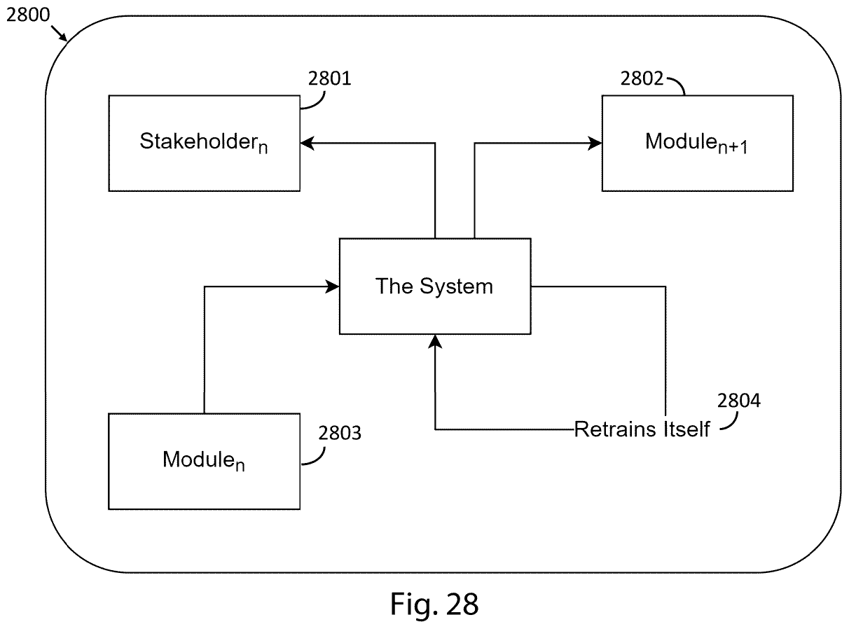

The system of the present embodiments is a cyber-physical system (CPS) aimed to provide the healthcare industry the capability of operating at peak-efficiency and improve patient outcomes. As shown in FIG. 28, the system 2800 connects all industry stakeholders 2801 to real-time data sets in a live environment to redistribute resources and increase operational efficiency. The system connects to its own designated service modules (suite of applications) 2802 along with third-party applications 2803. This enables bi-directional, tri-directional, quad-directional, and nth-directional data sharing for all stakeholders 2801 in the healthcare industry, using the emergency medical services (EMS) as the central fulcrum for operations. This system is designed with the healthcare system in mind, providing a solution to operate at maximal efficiency while under extreme stress. For example, when the availability of resources becomes scarce, supply chains break down, and surges in the number of patients exceed the available amount of personnel, and supplies needed for safe operations.

The system of present embodiments refers to a plurality of modules, and its relationship with the system can be a single software application 2800 on FIG. 28. The System can provides three main functions: (i) to distribute data which is obtained from all the modules 2802 and 2803 to each stakeholder 2801 in real-time, (ii) to redistribute data which is obtained from all the modules to other modules in real-time, and (iii) to retrain its own ML models in real-time 2804. Each module provides two purposes: (i) to provide a specific application (product) for a targeted solution, and (ii) for the application to provide a source of sensors (data gathering) for the system to train itself in real-time within the specific healthcare systems environment in which it is implemented and redistribute its data accordingly, either to a (i) stakeholder 2801, (ii) back to the system itself 2804, or (iii) another module 2802 (see FIG. 28).

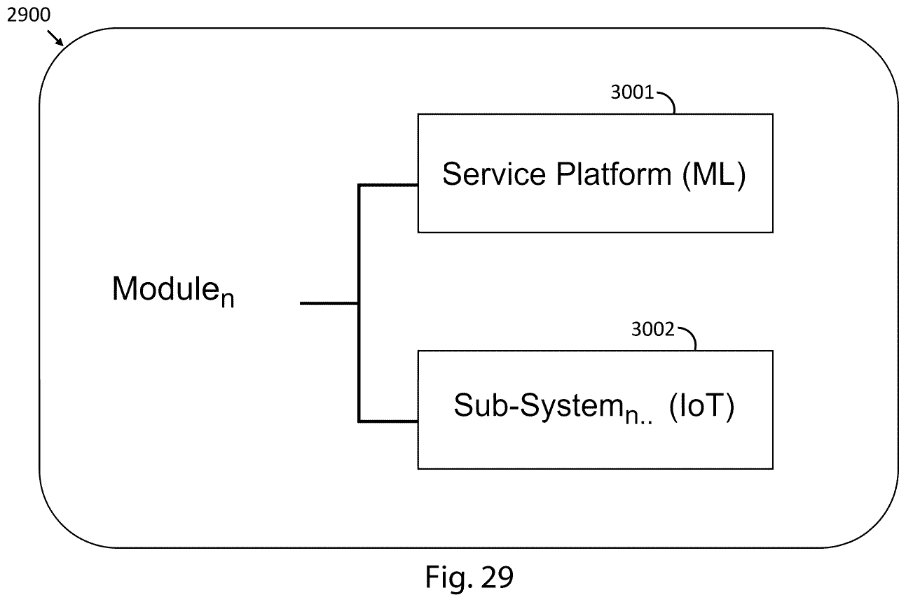

Each module 2900 has an IoT platform 3002 and/or a single ML model or group of ML models 3001 to achieve its designed purpose for the user (see reference 2900 on FIG. 29). The IoT platform 3002 is described as sub-system ‘n’ (IoT platform) and the service platform 3001 describes the ML model of group of ML models. Each module serves as an application to provide the system with a real-time data set of the healthcare environment in which it is implemented in (see FIG. 29).

To understand the information flow and analysis of this system one must understand the metrics that determine patient outcome in the EMS environment. What is observable about a patient outcome includes things checked or measured on the patient, such as temperature and oxygen blood levels, and the environment where the patient finds him- or herself. This is the state space of the patient 5500 in that environment and the patient trajectory 3100 is nothing more than the aggregate of all these factors' current state. This status is the only thing measurable and make decisions based on, in the hope that those decisions push that status toward the best possible outcome.

Based on some of the learning in EMS, EMS agencies and hospitals around the country are recognizing the value of bidirectional data sharing among healthcare organizations to optimize patient outcomes. Access to critical information in real time is paramount in yielding better patient care and improved performance for all involved entities, both medically and business-wise. This allows stakeholders and entities in the medical field to make informed decisions, allowing for an extended and integrated treatment window, i.e., after the onset of symptoms and incl. all components of the care. Thereby, shared data can provide information on success stories regarding positive patient outcomes (e.g., through the use of metrics and ML). Public access to information on the other hand, could potentially allow patients to make better decisions about such things as where to live to have better healthcare services. Lastly, appropriateness of destination should go beyond ‘nearest destination’ to include destination capabilities. Based on these learnings the system can develop metrics that approximate the factors called out. These include metrics such as information transfer times, entities' KPIs 3301 and 3302, number of entities involved that have existing access and capabilities (and use the data), condition-based metrics against treatment KPIs, improved treatment KPIs over time (x-day/month running calculation) etc.

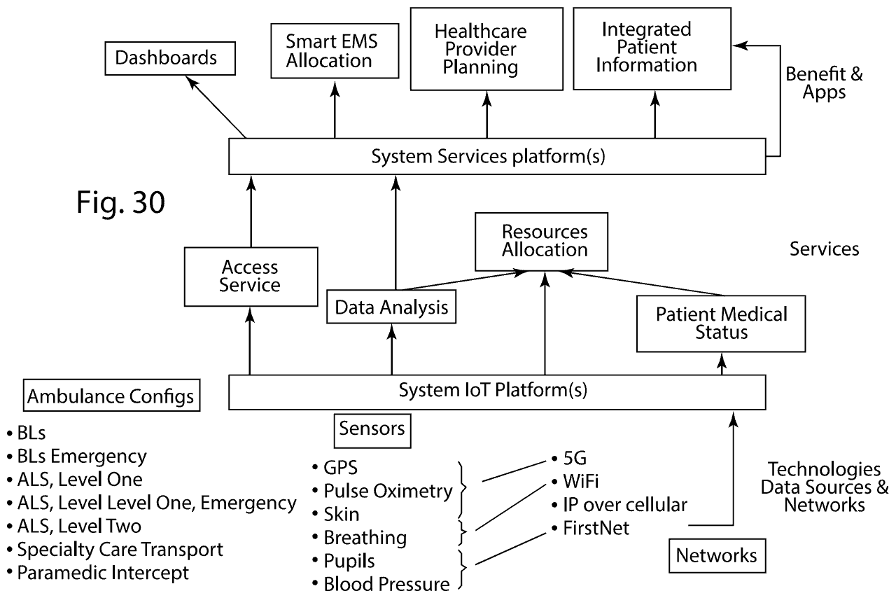

To address the need to make good decisions, data sources can be represented, calculations needed to understand the data and the desired benefits of tools that integrate the healthcare components as a set of information flows between an IoT platform 3002 and a service platform 3001 (see FIG. 30). This is intended to inform system development, including requirements specification, and helps in determining an assurance strategy, e.g., compliance related requirements. As indicated the IoT platform 3002 receives data from various sources including sensors 3003, databases 3004 and networks 3005 (see FIG. 30). Thereby, the mode of transmission may vary depending on the source or device data is received from. Sensor 3003 and data sources obtaining and measuring patient related data such as heart rates, breathing rates, pulse oximetry, O2 saturation, blood glucose levels, skin color, pupil size, mental status etc. as well as non-patient data including GPS and location data, are transferred via suitable networks 3005 including Wi-Fi, 5G, FirstNet or in person over cellular contact. On the other hand, ambulance configurations designed for different call types such as BLS, ALS, special care transport, paramedical intercept etc. are obtained from the EMS agency database 3004 and are accessed via the IoT platforms API.

As information exchange occurs the IoT platform 3002 reuses data for different services depending on the given service module (see FIG. 30). This ranges from simple access service modules 3006, where information is displayed 3007 to Hospital staff, EMS responders or EMS dispatcher for patient or device monitoring, to more complex services modules such as for condition-based resource allocation 3008, which requires integration of several data sources 3009 and extended computing and data analysis 3010. Other examples of information reuse include historical data analysis of correlated conditions (e.g., between heart disease and respiratory diseases) and data visualization such as an incident map based on patient condition categories e.g., trauma and other incident clusters or incident location and distribution.

The service platform receives 3001 information output from the service modules and directs it where services are needed (see FIG. 30). Based on the prioritized goals and outcomes the resulting benefits aid in a number of applications such as smart EMS allocation 3011 based on patient history and needs, hospital or clinic investment strategy or adaptive planning 3013 such as in the advent of national emergencies like the COVID pandemic, integration of hospital-EMS patient information 3012 to create integrated data naming conventions or for life data feeds from patient during transport to name a few.

In general, the system enables a number of different solutions to take shape in the medical field. Data-driven resource allocation based on need profiles and/or need predictions is accomplished using comprehensive patient history and ML algorithms to predict demands in each service area and scores of patient outcomes. Endured resilience to surges and variation in need can be buffered when demand deviates. As information about patient condition and resource/type of skill needed to perform certain tasks in patient management becomes available, coordination of hospital resource availability allows for efficient and adaptive resource management and greater utilization of human resources, including reuse of skills and cross-task allocation of personnel. Moreover, measured results can be integrated into holistic KPI metrics which stakeholders use to evaluate for instance efficient use of EMS as communications network, EMS density function aligned with predicted demand, smartness metrics etc. Another service application made possible by the system is, EMS as a Healthcare Network Management System (EHNMS). EHNMS allows EMS services to operate as a federated entity, to adapt to nationwide changes in need (volume and type) and provide the link between unconnected healthcare system components. The systems interoperable connectivity between EMS and hospitals, allows EMS and hospitals to operate as a single federated entity under Healthcare as a Network Management System (HNMS). HNMS enables EMS and local hospitals to work together in adapting to nationwide changes as well.

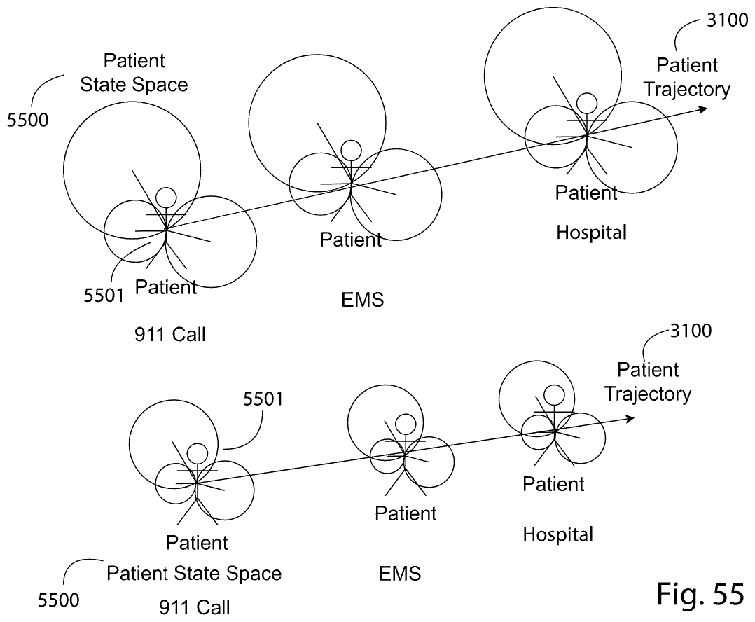

The patient state space (see 5500 on FIG. 55) is defined by the point in time at which the patient will seek medical attention, and the interaction between the patient and all stakeholders, care givers, supplies, tasks, and personnel which will be involved directly or indirectly to accomplish the full delivery of care. The delivery of care is generally described as the coordination between healthcare providers and administrators who work to provide quality care to patients. For this purpose, this definition is expanded to include all members involved in coordination directly or indirectly, such as medical manufacturers, logistics companies, billing personnel, etc.

When the Patient Condition Profile is generated 5806, the system will continuously optimize the delivery of care within the patient state space 5500 along the patient trajectory 3100. This includes provisioning of resources and communication between all members involved in the care of the patient directly and indirectly. The system will shorten the patient trajectory, while optimizing the delivery of care and coordinate with all members of the patient state space 5500 to provide better patient care and outcome. The system will optimize tasks involved around the delivery of care, inc. supply provisioning, care giver provisioning, processing of billing claims, communication between caregivers, and the like.

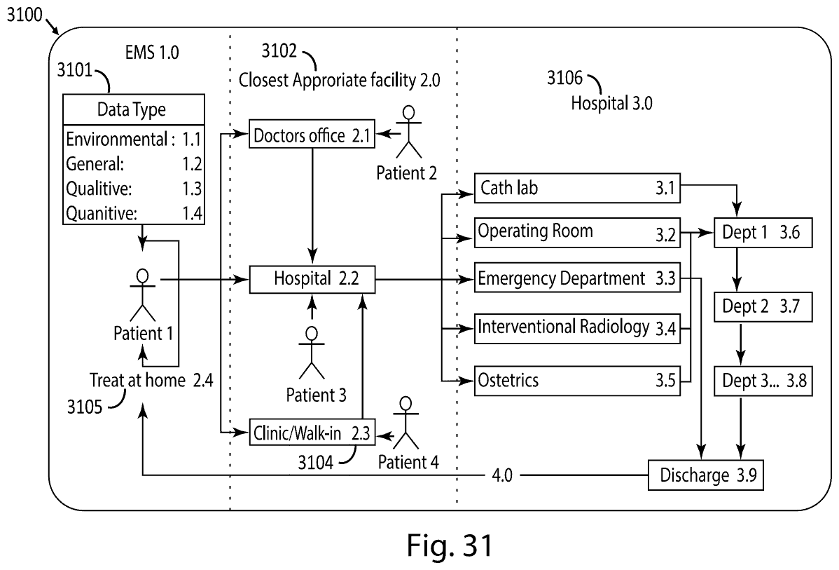

The patient trajectory (see 3100 on FIG. 31) is determined by the system to find and suggest the shortest path with greatest patient outcome. The system calculates the most appropriate path by determining the patient condition profile (PCP) 3200 in order to identify what the optimal treatment method and destination is. The data Type 3101 which are obtained during the EMS call include Environmental Data (weather, altitude, latitude, longitude etc.), general data (dispatch call type, time, date, day of the week, holiday, special events, etc.), patient related qualitative data (primary assessment, secondary assessment, focused physical exam etc.) and quantitative data (Lab values, EKG, USG, PVID, x-ray, CT-scan, MRI, etc.). Then the system analyzes the surrounding closest appropriate facility 3102 and compares them to the facilities real-time KPI's 3302, including bed availability, staff availability, available resources, time-to-treatment and the like. In order to determine the shortest patient trajectory. Thereby, destination decisions are determined by the location of the closest appropriate destination 3804 and by regional protocols/policies and other criteria. For instance, the patient may be transported to a doctor's office 3102, primary care physician, specialty physician, a clinic or walk-in 3104 a hospital 3103 or (depending on local capabilities) may stay and be treated at home 3105. Depending on the severity of the patient condition a special resource center 3106 may be required including obstetric center (patient is suffering from OB related complaint, greater than 24 weeks pregnant), stroke center (patient is suffering from suspected CVA/TIA), trauma center, (patient is suffering from trauma), STEMI center (patient is suffering from suspected/confirmed STEMI) or pediatric ER (depending on the region where specialized pediatric ER's are available, local protocols will dictate that all pediatric patients are transported to an ER, however each respective hospital has different requirements for the cut off age for patients who are to be treated in the pediatric ER). The transport decision is furthermore enforced by the patient medical records. For example, if a patient is suffering from a chronic issue and is already receiving specialty care, the decision will be made to transport to facility under the umbrella of the specialty care physician.

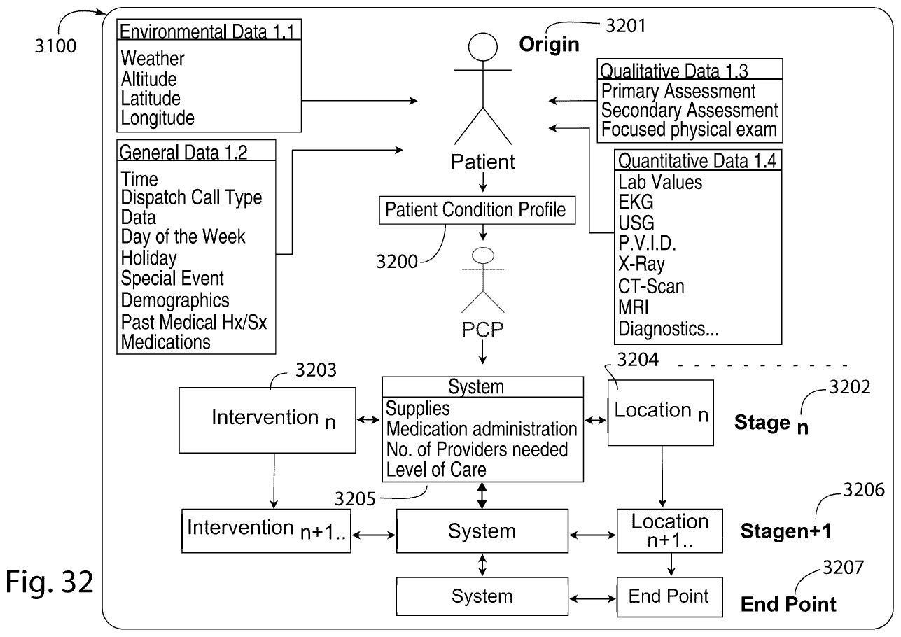

The Patient Condition Profile (PCP) (see 3200 on FIG. 32) is determined in real time by collecting assorted data from various sources, such as video data, audio data, diagnostic devices, and the like. From the origin 3201 (see FIG. 32/Origin) the system calculates the patient condition profile and determines the appropriate trajectory for the patient. The patient is either treated at its origin or transported to another location, for example a hospital, doctors office or walk-in clinic (see FIG. 31). At the decided location of treatment 3202 (see FIG. 32/Stage n) the system is comparing in real time the patient treatment, supplies used for treatment, Interventions used for the patient, the number of providers needed, the level of care, and the like. At each stage of patient movement in its trajectory 3206 (see FIG. 32/Stage n+1) this process is repeated until the patient is discharged form the last facility of care or dies 3207 (see FIG. 32/End point).

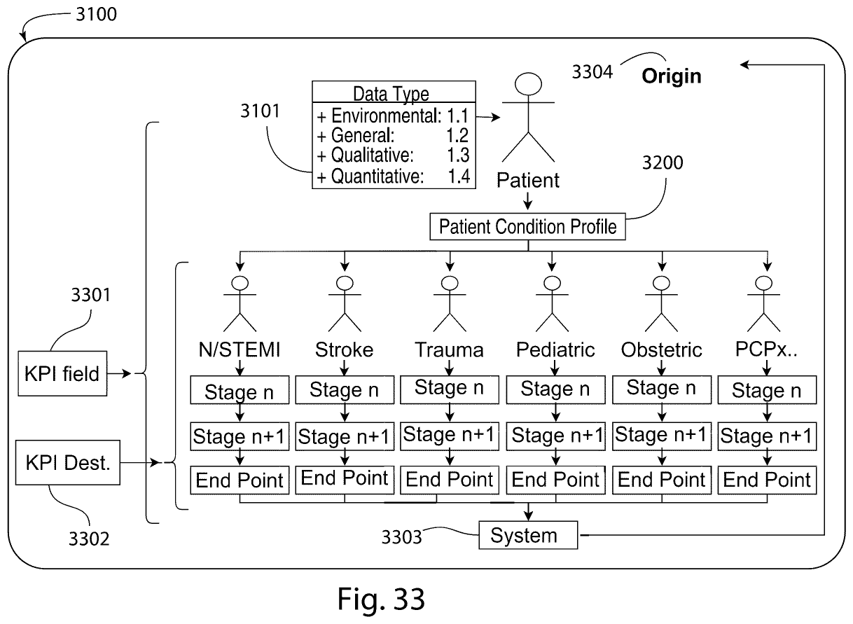

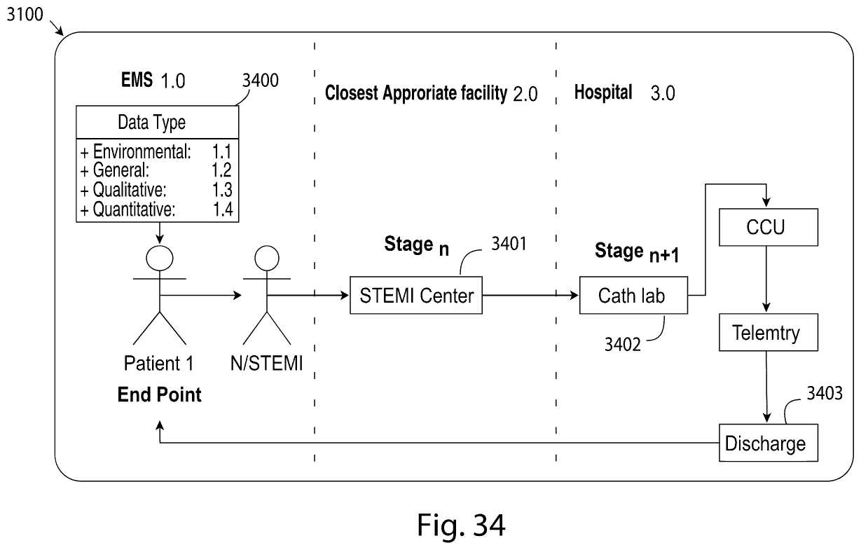

The patient trajectory with the correlating patient condition profile is compared against the collective field KPI of all surrounding facilities 3301 (see FIG. 33/KPI field). Once the location of treatment is determined, the system compares the destinations KPI 3302 (see FIG. 33/KPI Dest.) to optimize in real time, patient staff ratios, supply usage, bed occupancy, decrease waiting times and the like. The system organizes each PCP in real time for the most optimal outcomes. The system 3303 then compares the endpoints to learn the most likely origination point 3304, for the next incident (see FIG. 33). To give an example for a specific patient condition profile FIG. 34 shows the patient trajectory 3100 of a patient who meets STEMI criteria or a suspected NSTEMI. As illustrated earlier, the PCP is determined using data obtained from the EMS environment (see 3400 on FIG. 34) and the appropriate destination is decided which in this care is a special care facility for STEMI patients 3401 (see FIG. 34/2.0). Protocol demands the patients to be transported immediately to the Cath Lab 3402 for angioplasty and stent placement (see FIG. 34/3.0). After initial treatment the patient is transferred within the facility for further observation for observation until discharge 3403 (see FIG. 34/3.0).

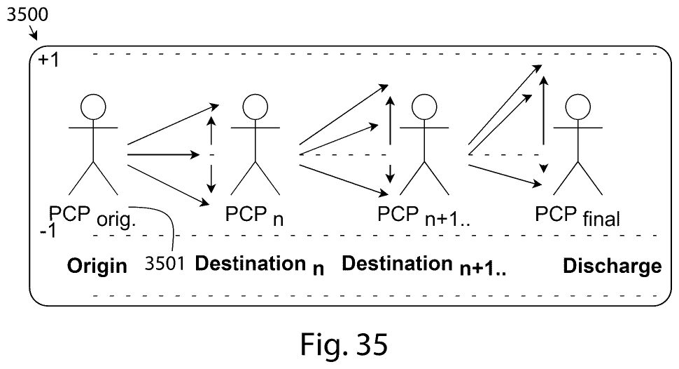

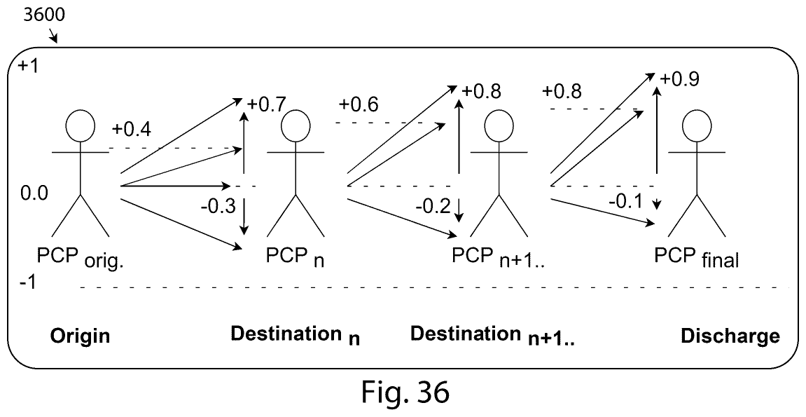

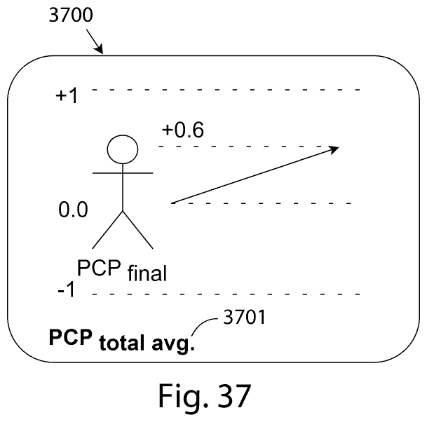

The system at each stage is able to obtain a new KPI (see 3500 on FIG. 35) which is a value between +1 and −1. +1 is the most improved and −1 is the least improved. The PCP 3501 is compared to itself from each previous stage. Improvement is measured by visual and audio data, diagnostics, and the like. Just as a provider forms a general impression of patient improvement by taking in patients' condition, vitals, appearance, and what the patient says, so does the system take in all the surrounding information and form a value between +1 and −1. Just as when a provider sees negative results with positive results simultaneously and makes an overall judgment whether the patient is improving or not, so does the system in adding the two values to determine a PCP value for each stage (see 3600 on FIG. 36). The average of all PCP values 3701 at the end of the patient trajectory is calculated and used to measure the systems effectiveness in patient outcome (see 3700 on FIG. 37).

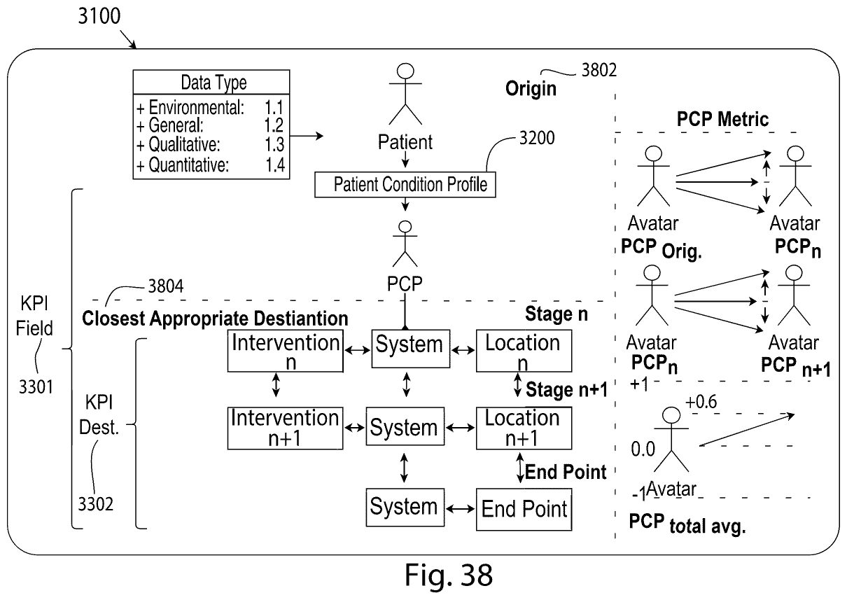

FIG. 38 demonstrates the optimal patient trajectory against the field 3301 and destinations KPIs 3302, with measured patient improvement each stage. As previously stated, the system calculates the PCP 3200 at the origin 3802 and compares it against the collective field KPI 3301 to determines the appropriate trajectory for the patient. The patient is then transported to the closest appropriate destination 3804 of treatment while the PCP continues to be monitored and compared to the destination KPI 3302 to optimize the patient's trajectory and direct it to the most optimal outcome.

The system of the present embodiments includes a number of programming technologies such as Python (possible frameworks: PyTorch, Tensorflow, SciKit) for machine learning, Pandas and Spark for data analysis, and Python and JavaScript (e.g., data visualization libraries: Plotly, D3.JS) for data visualization. The reason the above mentioned represent the right choice for the application is to easily identify hidden patterns and trends in datasets. By identifying these patterns and trends in datasets demand-response problems to optimize running costs can be solved, eliminate possible bottlenecks to improve response time. Also, it also helps medical personnel in decision-making tasks to improve the decision-making process for making faster and more accurate decisions.

The system of the present embodiments uses regression algorithms which are a subset of supervised learning algorithms to do call volume forecasting, prediction, and supply usage modelling. These algorithms predict the output values based on the input features (e.g., date, time, location, call type, . . . ) from the data given into a trained model. The trained model does a generalization on the training dataset (labelled) to model dependencies and relationships between input features and target output (label). Furthermore, the system uses regression trees and lasso regression algorithms for discrete dataset and neural network for continuous and more complex dataset. These algorithms are combined with the ensemble learning methods to improve the predictive performance.

Contextual multi-armed bandit algorithms are used for resource allocation and decision-making problems. Moreover, Bandit algorithms are mostly used for recommender systems such as personalized ads. RRBandit algorithms can be seen as a special case of Reinforcement Learning (RL) algorithms. Their goal is to maximize their overall reward by trial and error. These algorithms deal with the exploration/exploitation trade-off where a RL agent explores new actions to try to find the optimum action or exploits its knowledge to pick the best action that yielded the best results so far.

For continuous state (time series) and action resource allocation problems Deep Reinforcement Learning algorithm are used specifically Soft Actor-Critic (SAC) algorithm. The SAC algorithm ensures a sample-efficient and stable learning where state and action spaces are continuous (e.g., state space: patient condition during transportation, and action space: medical treatment). Furthermore, SAC algorithm is a model-free RL algorithm which means it does not use a model of the environment. This allows the trained RL model to adopt different environments.

The reason why discrete datasets are used for regression algorithms (supervised learning) is because they are faster to train, and the training requires less amount of data compared to deep learning algorithms. In addition, it can be challenging to tune the hyper-parameters of the deep learning algorithms. However, care is needed with some of the supervised learning algorithms since they are used to do generalization based on the training dataset, and these algorithms when they encounter bias in the input data (unseen, noisy or outlier data point), they tend to give an inaccurate output.

For more complex problems and continuous datasets, neural networks are used since they can model non-linear and complex patterns. Also, they can find hidden patterns where data volatility is very high and non-constant variance.

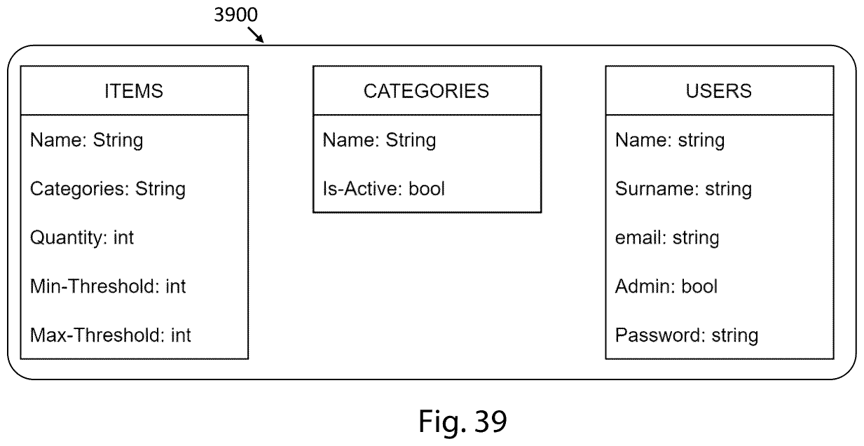

For the Backend (see 3900 on FIG. 39), the system uses the following programming technologies: GoLang for the backend Programming Language, BoltDB for the database and Kubernetes as the orchestration tool. Currently, there is a restriction for the Kubernetes version of cloud server providers. For that reason, the program utilized Google Cloud Platform.

The system of the present embodiments uses a schema that describes the attributes and analytics used in the pre-and in-hospital environment. The schema encompasses attributes of a record in either XML and/or JSON format. The analytics which are represented by the names for specific queries and reasoning over those names i.e., inferences and logic used to reason about the patient records) include the raw data, names such as conditions defined by a range of values for selected attributes/physical exam data) and associations of human elements such as patient, EMT, doctor, paramedic, etc. The schema also includes decision trees for each of the possible conversations, e.g., disease/illness script and resulting trajectory for the patient.

The following attribute and analytics for EMS are included in the schema, but are not limited to: the simulation (collecting and storing the entire simulation (see Module 1)), connections to smart city data (traffic light patterns, quickest route from destination, GPS location of dispatch unit, GPS location of proper destination), EMS call data (time, date, location, latitude, longitude, time of year, weather, altitude, holidays), traffic data (distance to destination, travel time, expected time of arrival, actual time of arrival), EMS dispatch call type (e.g. cardiopulmonary case, trauma case, pediatric case etc.), dispatch call priority (basic life support, advanced life support), time of first contact, time of intervention/treatment/medication, time of transport, chief complaint (such as chest pain, difficulty breathing, pain from fall etc.), patient complaints (verbal description of patients such as ‘my leg hurts’, ‘my chest hurts’, ‘I feel like a can't breathe’ etc.), primary provider impression (patient unresponsive, STEMI, COPD etc.), secondary provider impression (head injury, syncope etc.), vitals of the patient (heart rate, blood pressure, respiratory rate, SPO2 level, blood glucose, EtCO2, CO level, temperature), EKG rhythms (sinus rhythm, junctional rhythm etc.), 12-lead reading (left bundle branch block, left ventricular hypertrophy etc.), cerebral perfusion pressure, vent settings (including modes such as pressure regulated volume control, assist control and pressure support as well as tidal volume, pip (peak inspiratory pressure), PEEP etc.), EMS provider differential diagnosis (COPD, STEMI, stroke, fracture etc.), transport destinations such as to closest hospital or to specialty resource center (cardiac center, trauma center, pediatric ER, Obstetric ER, Burn center, stroke center), disposition report (RMA_refusal medical assistance may also be seen as AMA, transferred care to ALS, transferred care to BLS, transferred care to helicopter, patient ran away etc.), oxygen delivery method (nasal cannula, simple, air cushioned mask, partial rebreather, nonrebreather, venturi including F102% settings, CPAP including value in mmHg, BiPAP with IPAP (inspiratory positive airway pressure) and EPAP (expiratory airway pressure) and F102%), Medication given to the patient (e.g. Epinephrine with dose and rout of administration like intra venous IV, intramuscular IM etc.), airway & breathing equipment (OPA—oropharyngeal airway, NPA—nasopharyngeal airway, laryngeal mask, laryngeal tube with size, king airway with size, I-gel, endotracheal tube with size, tracheotomy, CPAP, BiPAP etc.), circulation management (normal saline in either 0.9% or 0.45%, lactate ringers, blood type including Type A/B/AB/0/Rh+ or −, plasma), cardiopulmonary resuscitation time and intervals, electrical therapy, and cardioversion procedures (with manufacturer specification like Philips, Lifepack 15 and Zoll and method such as synchronized electric cardioversion with dose (joules), unsynchronized_or defibrillation with dose (joules), transcutaneous pacing with dose in ma (milliampere) etc.).

The following attribute and analytics for Hospitals are included in the schema, but are not limited to: admitting diagnosis, logistics (time at ED, balloon time, CT time), diagnostics (such as 12-lead, stress test etc.), imaging (MRI, CT, x-ray, ultrasound), lab values (blood draw such as CBC, blood culture, urine analysis, spinal tap etc.), treatments (including medication administered, interventions, discharge location etc.), patient trajectory in the hospital (e.g. emergency department, catheterization laboratory, telemetry unit, discharge from hospital), admission date, admission length, length of stay in each unit, specialty care given (Cath lab, surgery, organ transplant etc.), discharge diagnosis, sensor values (such as O2 level, blood glucose, heart rate, EKG rhythm, vent settings etc.), patient demographics (including age, gender, weight, height, past medical history, genetic mutations, diseases, family history, cancer history, past surgeries, etc.), statements (patient interview, emergency medical dispatch script), assessment ID, subjective assessment (dizziness “I feel dizzy”, weakness, difficulty breathing “I am having trouble breathing”, “I can't breathe”, nausea & vomiting, etc.), pain (with description of location, radiation, character, duration, frequency, reliving factors), patient orientation (to person, to place, to time), Glasgow coma scale, airway assessment (patient, self-maintained, obstructed), breathing assessment (lung sounds like wheezing or crackles, intercostal movement, labored respirations, stridor (check sometimes in airway, sometimes in breathing), tripod position, difficulty breathing, etc.), circulation assessment (heart tones, heart rhythms, pulse, etc.), disability (neurological status), exposure (visual appearance), skin color (pink, pale, cyanotic, mottled, lividity), skin temperature, skin condition (diaphoretic, clammy, dry), skin turgor, physical exam (deformities, contusions, abrasions, punctures, burns, lacerations, tenderness, instability, crepitus, head examination, neck examination, chest examination, abdomen examination, pelvis examination, and extremity assessment), diagnosis, parent group, signs and symptoms, supplies (call type, name, number, reusable, expiration date, criticality, cost, restock, amount) and discharge diagnosis.

The system of the present embodiments provides a method to coordinate an intelligent EMS response to improve quality of care, resource provisioning and preparedness to variation in patient demand. Starting with the emergency medical services, the system of the present embodiments allows healthcare systems (as well as supply and pharmaceutical manufacturers) to operate as a federated entity which allows decentralized healthcare units to work together in close cooperation in a centralized manner. Thereby, the system improves the quality patient care and healthcare services by accomplishing three main tasks: i) allocate resources at the patient side to meet patient demand, ii) optimal patient trajectory relative to duration of care or quantity of steps in the trajectory, incl. steps involving institutionalization iii) provide decision making support for care providers to improve quality of care for the patient.

The system of the present embodiments uses its various modules and connected applications as extra sensors to obtain large datasets in real-time, simulating a real-life environment. The system reads the data given to train multiple ML models. The output of the ML models will be configured in multiple ways to run the simulation specified by the user. The simulation ran by the ML models is then stored in the database of a given module, to save space and store unlinked data to adhere to data privacy laws such as HIPAA and GDPR.

The system unites EMS dispatch centers, care providers and supply distributers to work together to better meet patient demand and improve quality of patient care. The system provides continuous analysis of three profiles to provide the optimal patient trajectory and quality of care throughout the care track. The three profiles are the EMS Unit Profile, the Patient Condition Profile (or Patient Care Profile), and the Receiving Entities Profile. The analysis of these three profiles together results in improved distribution of resources, decreased burden on the healthcare system, and improved quality of care.

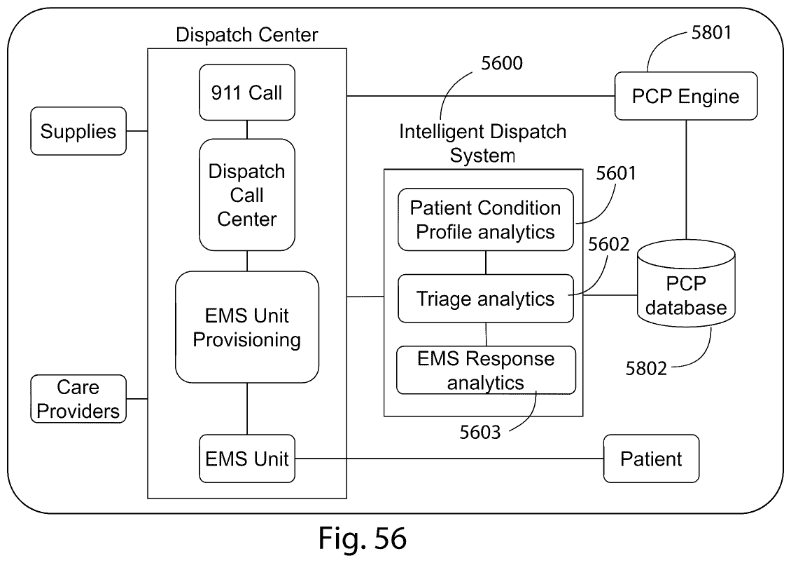

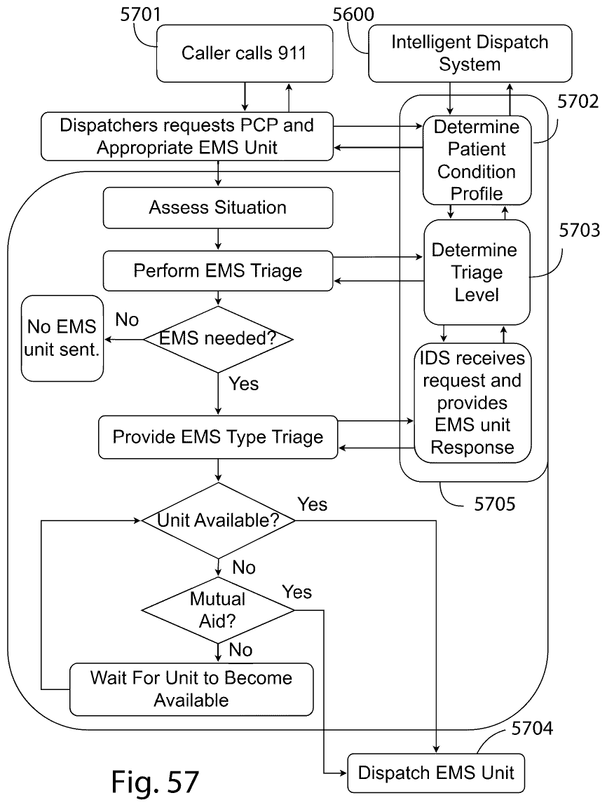

One method of the system (Intelligent Dispatch System) 5600 (see FIG. 56 and FIG. 57) performs analytics of Patient Condition Profile 5601, Triage 5602 (incl. patient criticality assessment), and Response 5603 to coordinate the outcome of the response. The patient care needs are based on the Patient Condition Profile and requirements of quality care, optimal outcome and supply and care provisioning around the changing patient condition. Triage inputs include Patient Condition Profile, to determine the optimal and appropriate EMS unit to respond and is performed by analyzing all the factors already discussed to determine optimal outcome, and provisioning of resources. The system applies its analysis of these components for the EMS response to design and coordinate EMS and Hospital daily operations and to prepare and optimize patient care treatment and outcome.

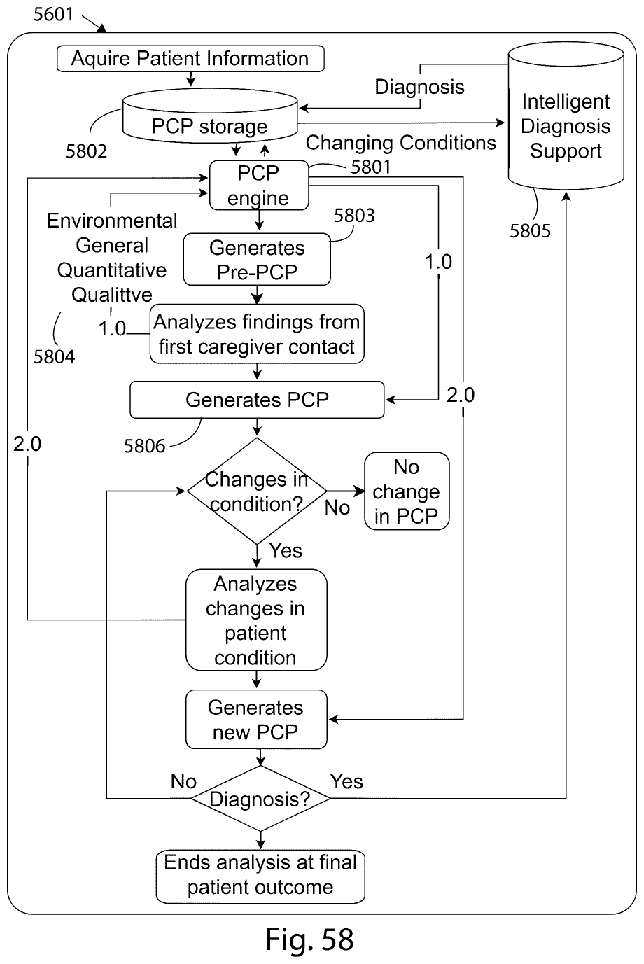

One method of the system (Patient Condition Profile Analysis) 5601 has two components: i) the Patient Condition Profile (PCP) engine 5801 and ii) the PCP Database 5802, which receives the information gathered from the 911-caller by the dispatcher and generates, stores and updates the Patient Condition Profile. The Patient Condition Profile is generated at the time of the 911 caller call and dispatch center (see FIG. 58). The content of the Patient Condition Profile is derived from the caller's answers to the 911 call taker's questions according to the conventional scripts (see FIG. 57). The situation is then assessed, and triage is performed by the dispatcher. In parallel the patient condition profile is continuously updated during triage and displayed to the dispatcher to aid in determining the right response based on characteristics of the patient condition profile. If it is determined that an EMS unit is needed, the closest available unit is then searched and once a unit become available, it is dispatched to the location of the 911 call. This method performs continuous intelligent analytics to adapt to changes in the EMS unit and receiving entity profile with relation to changing patient condition throughout the patient trajectory. Some inputs to this method may include patient assessment findings, complaints, point of care diagnostics, and the like.

The Patient Condition Profile (PCP) 3200 (see FIG. 32, 33, 38) is a set of attributes or characteristics which define a patient's health state at any given moment in the patient's trajectory. Throughout the course of the patient trajectory, form first contact in EMS to discharge from the place of treatment such as hospitals, PCP is continuously updated and analyzed. Results of this analysis serve to inform receiving entities and healthcare providers downstream of the patient's trajectory, aiding facilities in preparation of patient arrival as well as offering decision-making support for healthcare providers. Moreover, the change in the PCP informs supply chain logistics. The system of the present embodiments learns and analyzes the patient condition profile, to determine the trajectory based on patient outcome and time to treatment.

The patient condition profile is created by the Patient Condition Profile (PCP) engine 5801 and stored and accessed via the PCP Database 5802 (see FIG. 58). The Patient Condition Profile is generated at the time patient information is acquired. Depending on the environment the content of the Patient Condition Profile is derived from patients' assessment, patient report, 911 call taker's questions according to the conventional scripts, etc. An initial pre-PCP 5803 is generated by the PCP engine and compared to the findings of the first caregivers contact using environmental, general, quantitative and qualitative data. Once a PCP is established it is evaluated for changes and if the changes have been found in the patient state the PCP is again updated. Once the PCP offers clues to a diagnosis it is related to the PCP database where diagnostic support is given. PCP analytics is smart because it is adapting to condition change.

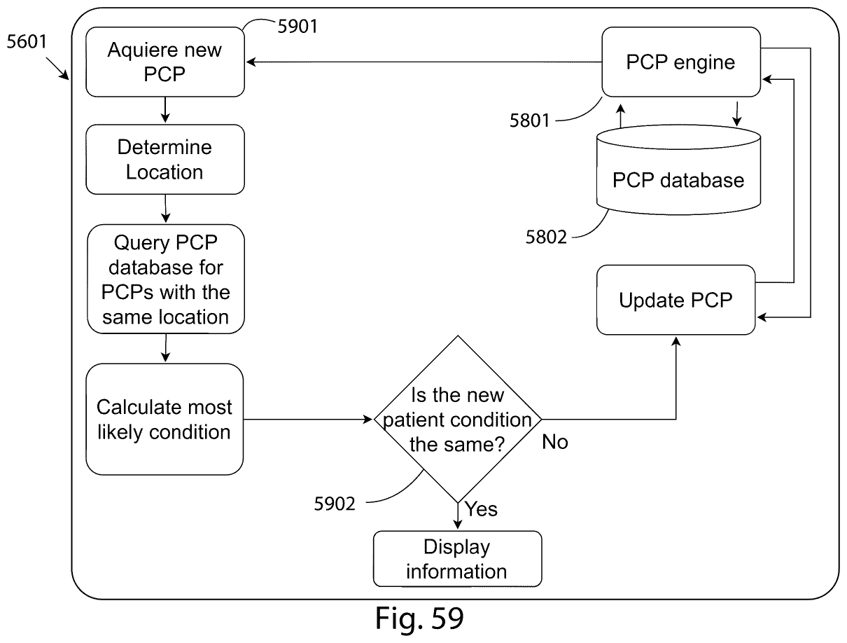

When a new location specific Patient condition profiles are acquired 4700, the system performs a query in the PCP database to search for PCPs with the same location (see FIG. 59). The system then calculates the most likely condition related to the PCP. If a matching condition is found, the information is displayed to the interface device, otherwise the acquired PCP is updated and added to the database.

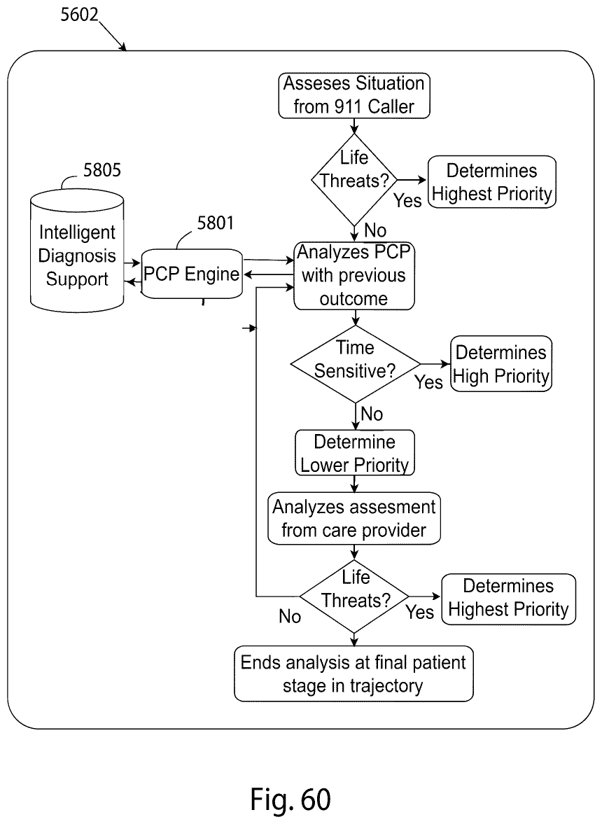

One method of the system performs triage analytics 5602 (see FIG. 60). First the situation of the 911 call is assessed, and priority is determined. If priority is low the PCP is analyzed to determine time sensitive of the condition. If low priority is determined the assessment is analyzed by the care provider for life threads. This process is continuing until the either high priority or final patient outcome is determined. Triage analytics is smart because it is optimizing outcome as a function of triage decisions in the past.

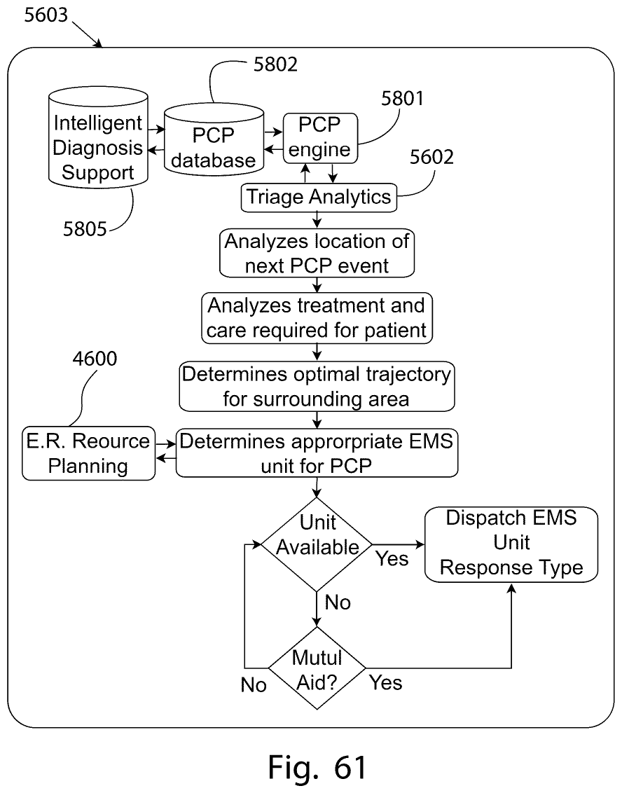

One method of the system performs EMS response analysis 5603 (see FIG. 61) which involves the interpretation of the patient condition profile analytics method and ongoing triage analytics method. The analysis between PCP analytics and triage analytics are some examples of inputs to this method. Some other inputs include, location of the call, time of the call, and the like. The output of the system is matching the proper EMS unit level (ALS or BLS) to be near the vicinity of the location of the call. Providing that the proper care can be delivered rapidly. This method provides Emergency Room coordination with the Emergency Room Resource Planning Method, providing Emergency Rooms to predict, prepare and plan for incoming patients for optimal patient outcome. Coordination with E.R. Resource Planning enables smooth patient delivery, care and supply provisioning to shorten the patient trajectory.

This method allows for the recognition of different EMS unit levels on top of ALS and BLS, which provide specialty care in the advancement of telemedicine. By recognizing different PCPs and locations, the difficulty of disbursing matching proper unit availability to every call opens discussions for specific cross training of providers, allowing EMS units to treat the patients at home via telehealth therefor decreasing ED overcrowding.

The system analyzes the EMS unit profile, ensuring the adequate level of care is available near the location of the emergency call. The EMS Unit Profiles may include Advanced Life Support (ALS) or Basic Life Support (BLS) unit types. The system analyzes the receiving entities profile for characteristics such as bed availability, staff availability and supply availability, along with predicted patient load by patient condition for that facility.

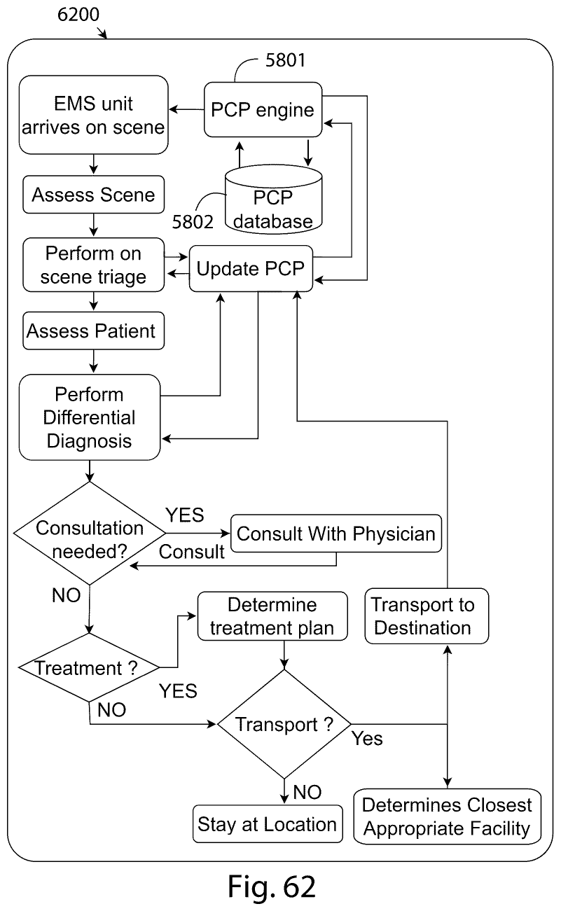

The PCP 5601 is continuously generated and updated 5805 throughout the entire patient trajectory. First patient contact is made when EMS first meets the patient during the EMS unit on-scene flow (see reference 6200 on FIG. 62). Once the EMS unit arrives on scene, triage of the patient is performed, and differential diagnosis are determined. At each of these steps the patient condition profile is updated 5801 and forwarded to the PCP database 5802. Once a treatment plan is determined for the patient and the closest appropriate destination is found the patient is transferred to the destination. Destination transfer of the patient is updated to the existing patient condition profile.

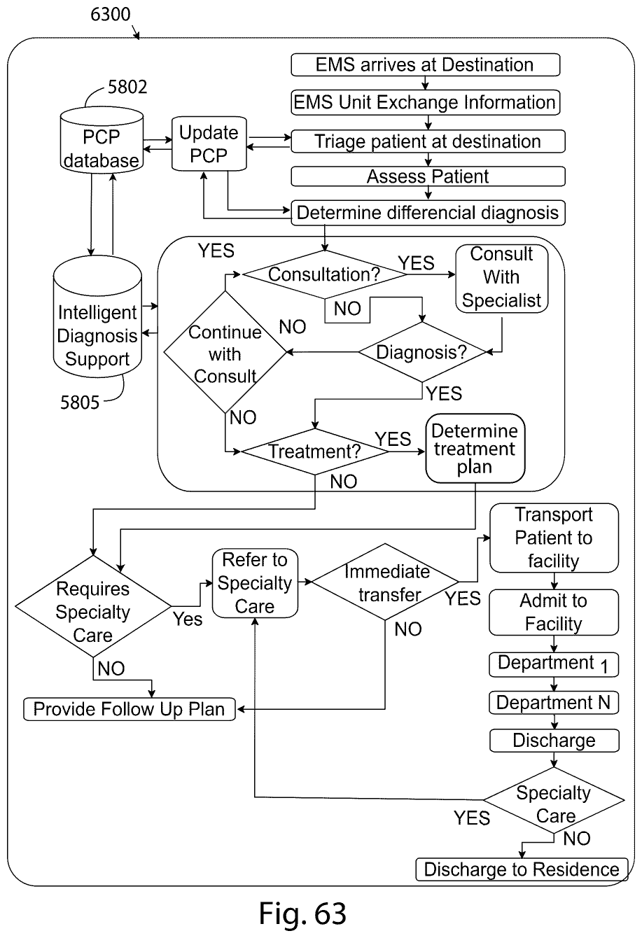

At the destination of the EMS unit (see reference 6300 on FIG. 63), triage and differential diagnosis are again performed, and the PCP 5802 is updated accordingly. The patient is then handed over to the consulting caretaker and a treatment plan is established with the help of diagnostics support 5805 with coordination with the PCP database 5802. If specialty care is required, the patient is transferred to the unit and follows the treatment plan until discharged. If no special consult is required a follow up plan is determined, and patient is discharged to proper destination.

The performance of the system will be assessed with an index or score. The lower the score the better the system performed. The score of a specific EMS trip is calculated by summing its scores in the individual factors and then dividing by the sum of the highest possible scores for each factor. If there is an order of importance between factors, then coefficients for the factor scores can be introduced to reflect that importance. Factors include PCP analytics (see reference 5601 on FIG. 56), triage analytics (see reference 5602 on FIG. 56), EMS unit response analytics (see reference 5603 on FIG. 56), stages in trajectory 3100, supply provisioning in trajectory, care provisioning in ED and ER preparedness (see reference 4600 on FIG. 61) (note that this list may be subject to change as factors may be added or subtracted). One example is the scoring system for the initial PCP vs actual diagnosis: if the diagnosis establish through the PCP is correct, it will receive a score of zero. If the diagnosis was incorrect but was able to narrow down on the organ system involved, it will receive a score of one. If the diagnosis was incorrect and did not narrow down on the organ system involved, it will receive a score of two.

The system and its components make possible different combinations of application services. Below is a list of some examples.

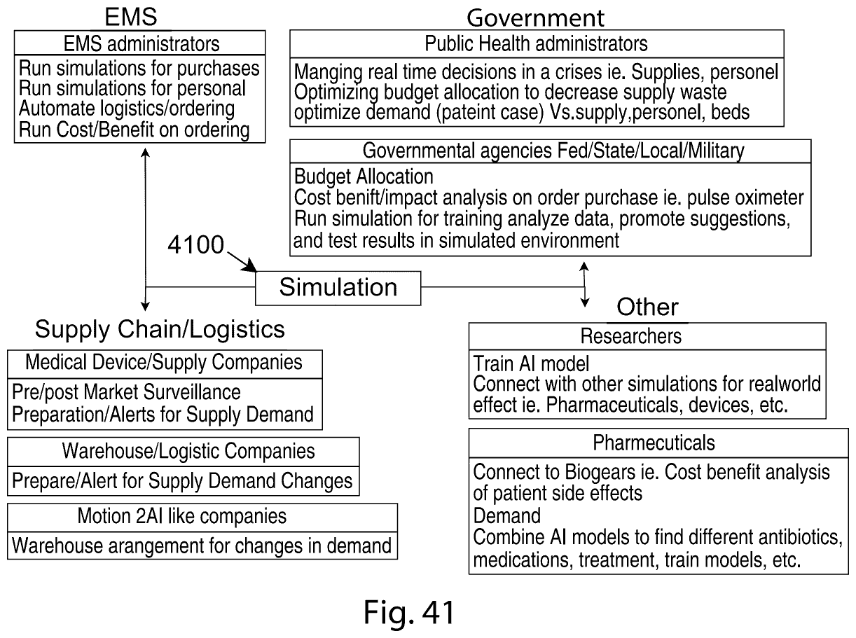

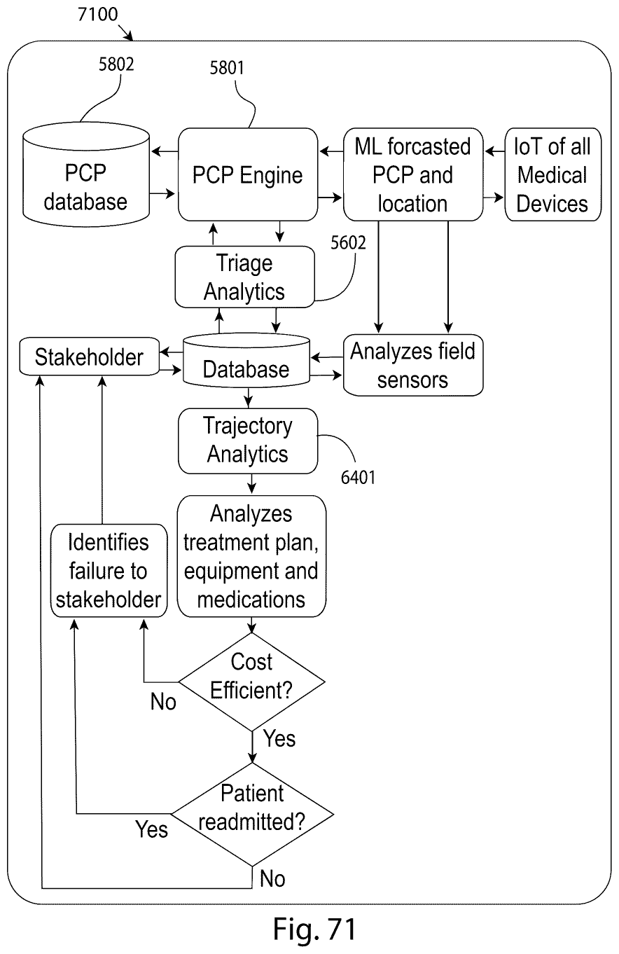

Module 1. Creates a simulated environment for the user from live sensors in the field. Sensors are collected from other modules, medical devices in operations, GPS monitoring, and manually inserted data as well from the user, if they wish (see reference 7100 on FIG. 71). The simulation will represent the environment specified by the user, either the hospital, EMS agency, or entire healthcare system in operations with multiple EMS agencies and Hospitals acting together. The module supports decision making for all users, ‘what if?’ scenarios, policy making, identifies inefficiencies in operations, highlights specialized training which make possible to distribute staff more appropriately, cost/benefit analysis, market surveillance studies, product demonstration, risk analysis, pharmaceutical study market analysis, field research and identifiers, and the like. (see reference 4100 on FIG. 41). The inputs will be the data gathered from all the sensors in the field, including medical devices, diagnostic materials, diagnosis, patient condition profile (PCP), the patient movement through various stages in the health care field, supply usage related to patient condition etc. The outputs are the answers to the questions posed by the stakeholder, for example an EMS manager may like to know, how long will my CPAP masks last, or a federal aid organization may like to know with a 5 million dollar budget which appropriate allocation of supplies should be sent (see reference 4100 on FIG. 41).

The healthcare ecosystem is made up of several entities, such as EMS agencies, hospitals, and clinics. Each operates independently and together towards the common goal for best patient outcomes, and to work together as a unified healthcare field. The method of the system connects to multiple field sensors it defines a real-life simulation, of each field, allowing each entity to operate independently or as a single entity.

The simulation environment provides the several functionalities: One functionality allows the stakeholder to simulate if in the institution or area of question, for example an EMS agency, the right number of supplies for a given period of time, for example until next month, are provided. Here, stakeholders have the option to select a specific time period (10 day, 20 days etc.) or to ask the program to run freely and simulate how long given supplies will last (results provided in days). To do that, the history of EMS call records of the institution or area in question is uploaded as well as the current inventory (number of supplies). Then, a time period is selected, and the simulation is run. Another functionality allows stakeholders to determine what inventory (supplies) at the end of a given time period (x days, x months, etc.) is left and which supplies are needed or should be ordered. Here, the only upload needed are the EMS call records of the institution or area in question before running the simulation environment. Moreover, extended simulation runs may include other parameters such as EMS transport routes and transport times, employee numbers, wages, purchase cost of new item etc. Stakeholders may use the results to adjust orders of scheduled transports, standby locations as well as number of ambulances used in the area, optimizing budget allocation to each entity to maximize efficiency. Afterwards the user has the option to run a cost/benefit analysis for new purchases in supplies or ambulances units, employee numbers and wages and shift distribution. In this way, the user has also the option to determine the priority of budget allocation and which purchases are most economical. Note that the EMS call records can be dynamic, and in an evolving state (patient condition state improves or worsens over time). Therefore, for the above-mentioned functionalities the simulation initiates a basic statistical analysis to find an average number of EMS calls per day and the distribution of each EMS call over a day.

Data points (Items/supplies) in the datasets are categorized using a schema with different attributes including interventions (such as IV access, airway intervention, suction etc.), names, numbers, expiration dates, usability of items (meaning single use items vs stationary items such as sterile dressings vs ECG monitors), durability of items (meaning the probability of items breaking or being rendered useless for further use), criticality of items (meaning necessities of infield usage (call dependent e.g. administration of crystalloid fluids in a cardiogenic shock patient), and cost.

There are several advantages over building a simulation platform into the systems infrastructure. As to lack of field-testing possibilities, testing ideas beforehand offer practical benefits in planning and execution before the real-world implementation.

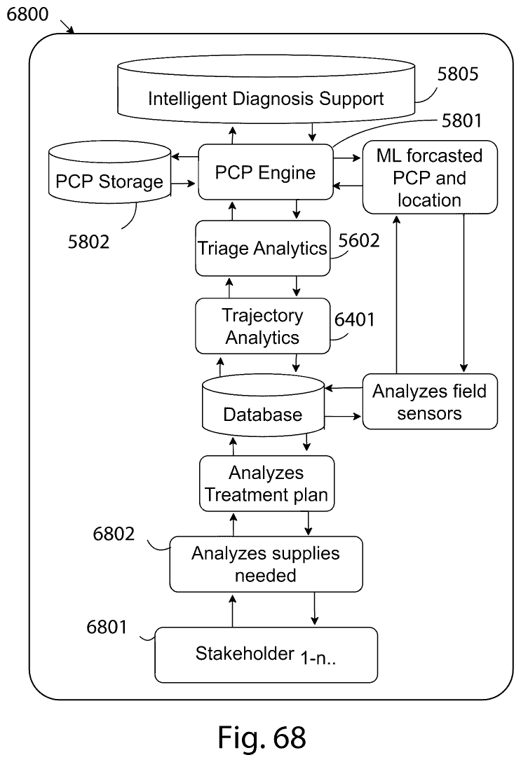

Module 2. Applies ML forecasting of emergencies (see FIG. 10) and events to automate schedule services (see FIG. 13), logistics, supply (see reference 6800 on FIG. 68), and supply ordering (see FIG. 14) across the entire patient state space (see reference 5500 on FIG. 55). This method of the system automates scheduling services based on optimal and safe personnel coverage and provides optimal number of emergency response units and locations based on need predicted by ML forecasting. The method of the system does autonomous ordering of supplies based on demand from ML forecasting of emergencies and events (see reference 6801 on FIG. 68). This method of the system runs cost benefit analysis on purchase items based on future use from ML forecasting. The method of the system focuses training based on ML emergency forecasting. The method of the system automates financial analysis based on future running costs and revenue based on ML forecasting which is a combination of supply provisioning and care provisioning. Inputs include the combination of the PCP and the outputs of the intelligent dispatch system, call location, weather, supplies used for the patient, etc. Outputs include call location, type, and the like.

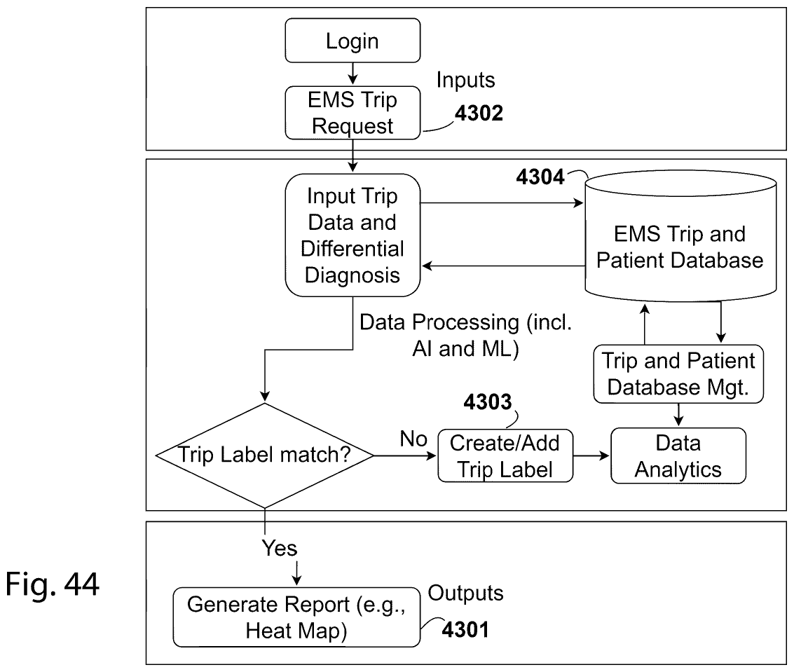

This method of the system (Module 2) (see FIG. 44) illustrates how EMS trip requests 4302 are processed and how new data is labeled 4303 within the EMS Manager assistant. Once an EMS trip request is sent to the system, the input data including differential diagnosis are searched within the EMS trip and patient database 4304. Once a matching EMS trip label is found a report is generated and the data is visualized in form of a head map 4301. If no matching label is found a new trip label is created and added to the data analytics.

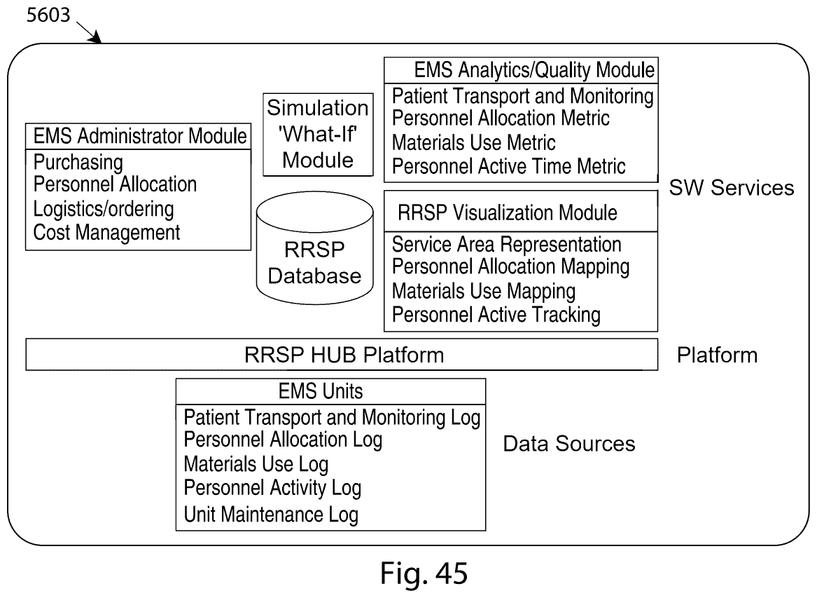

This method of the system (Module 2) (see reference 5603 on FIG. 45) illustrates the component of the EMS management services that are built on top of the systems platform and its related data sources. Provided services for EMS administration allow management of logistics and personnel allocation as well as management of purchases and cost calculation. Services for quality assurance and analytics management provide metrics for patient transport and monitoring, personnel allocation, and activity time as well as material use. Services for simulation allow to run what if scenarios to optimize resource allocation and test policy changes. Visualization allows for simple mapping and tracking of those services. The method of the system of the present embodiments includes a central database. Data sources from EMS units include information about patient transport and monitoring, personnel allocation, material use, personnel activity and unit maintenances.

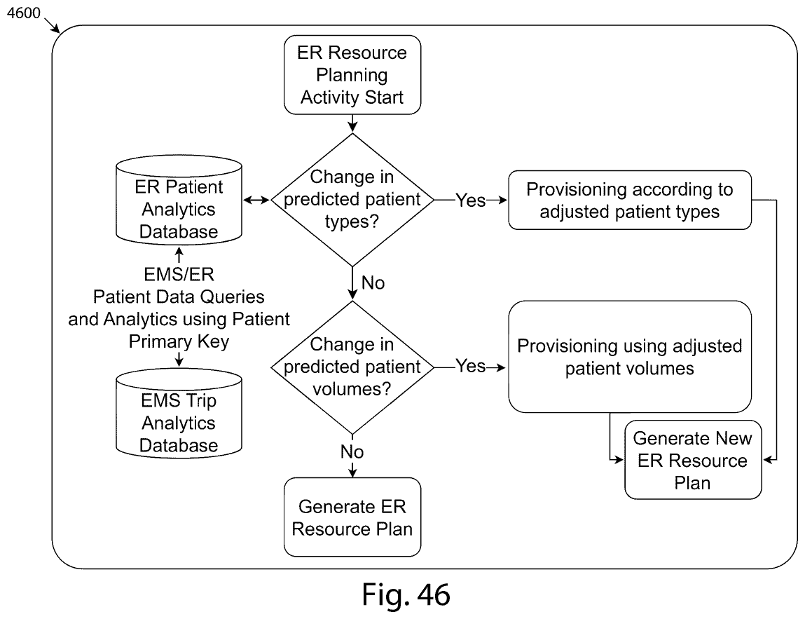

This method of the system (Module 2) (see reference 4600 on FIG. 46) illustrates how patient data analytics aids in the management of resource planning in Emergency rooms (ER). The method of the system analyzes patient data form EMS and ER queries and predicts changes in patient type (or patient cases) and patient volumes and adjusts provisioning including material and human resources to generate adequate ER resource plans. This method allows to increase preparedness and for efficient use of available resources in ERs.

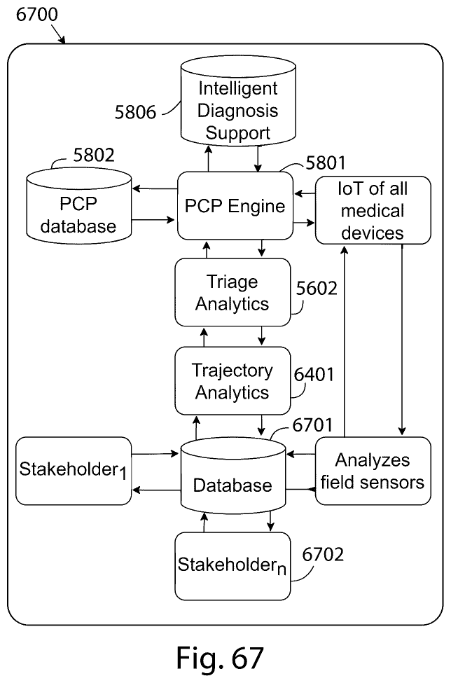

Module 3. Is a telemedicine platform which connects via internet of Things (IoT) all medical devices, across the entire patient state space and trajectory (see reference 6700 on FIG. 67). This method of the system provides suggestions for treatment, transport destination, and diagnosis. The method of the system sorts and packages mirror imaging diagnostics 6701 to the proper end-user 6702 for remote telemedicine services in the pre-hospital and remote care setting. The end user or stakeholder 6702 can be a support decision physician, other healthcare professional, or a documentation system to automate the documentation process.

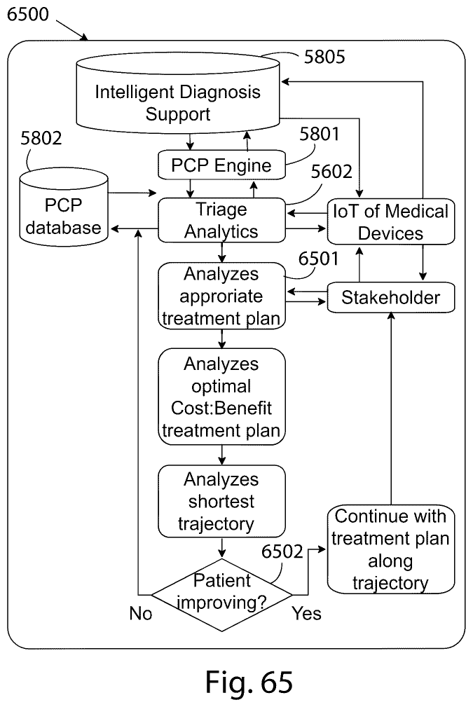

The method of the system (see reference 6500 on FIG. 65) suggests the optimal treatment plan 6501 and trajectory for the patient to the care provider. The method of the system learns the current patient condition profile 5801, and projected changes in condition from previous outcomes learned in the intelligent diagnosis support database. The method of the system continuously updates the treatment plan 6501 based on the current patient condition profile 5801, and critical level 5602 and predicted patient changes in condition (see reference 5806 FIG. 58).

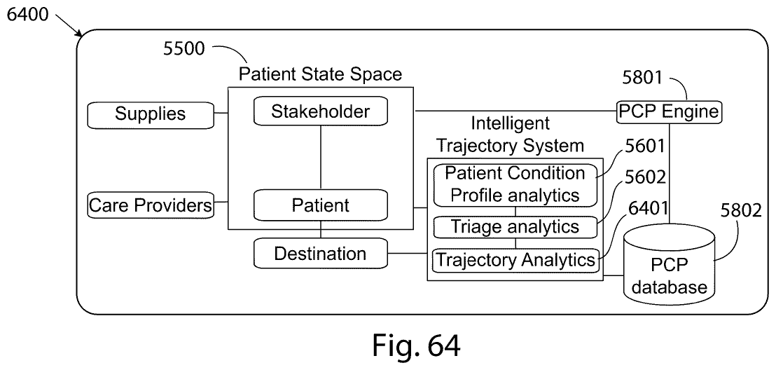

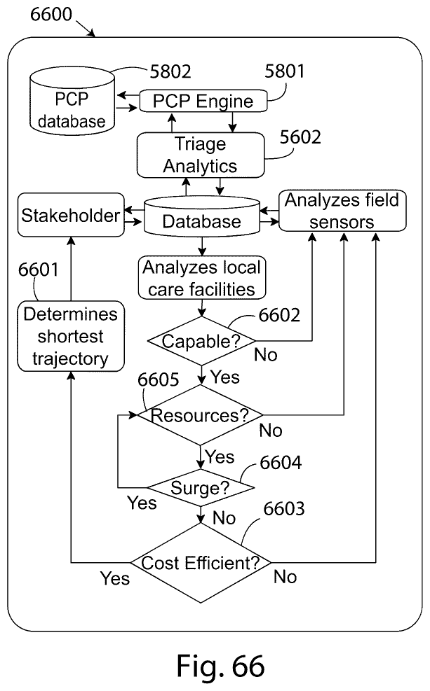

The method of the system (see reference 6600 on FIG. 66) analyzes the shortest trajectory 6601, and suggests transport destination, based on the patient condition profile 5801 and capabilities of the surrounding destinations 6602. The method of the system determines the optimal treatment plan based on patient outcome (see reference 6502 on FIG. 65). The method of the system then analyzes the projected changes in condition, criticality level, and analyzes the local destination capabilities, their resource availability, capability to handle current surges and other predicted patients entering the facility, and cost. The method of the system uses the input from all the surrounding field sensors, obtaining patient complaint, patient assessment, and other diagnostic findings from the IoT of medical devices to learn the predicted patient condition profile and optimize the shortest patient trajectory around the patient state space (see reference 6400 on FIG. 64).

The method of the system (see reference 6700 on FIG. 67) autonomously sorts and filters 6701 the necessary information to each stakeholder 6702. For example, the EMS care provider at the patient side is communicating with a telehealth physician. The necessary patient information and diagnostic information are filtered and sent to the telehealth physician. Each care provider will see the suggested treatment plan, diagnosis, and trajectory for the patient. This method allows for the sharing of all necessary information to each respected shareholder, not just in a telehealth setting. For example, if a medical manufacturer wants to run a field analysis of their device and impact on patient outcome, the system will filter and share the information necessary to the medical manufacturer.

Module 4. Intelligently coordinates operations between EMS and the emergency department. This method of the system receives diagnostic information and imaging (IoT) to present live to stakeholders, sort and properly triage, assign beds, and make suggestions for ER movement, increasing preparedness. If patient requires specialty resource care team, such as cardiologist, neurologist, trauma surgeon, diagnostic imaging will be shared and seen by team for early response care and preparedness. This output is possible based on the functionality of the method of the system (see reference 6700 FIG. 67) which analyses the PCP, treatment plan, trajectory, provisioning of resources, and criticality of all patients simultaneously to each stakeholder.

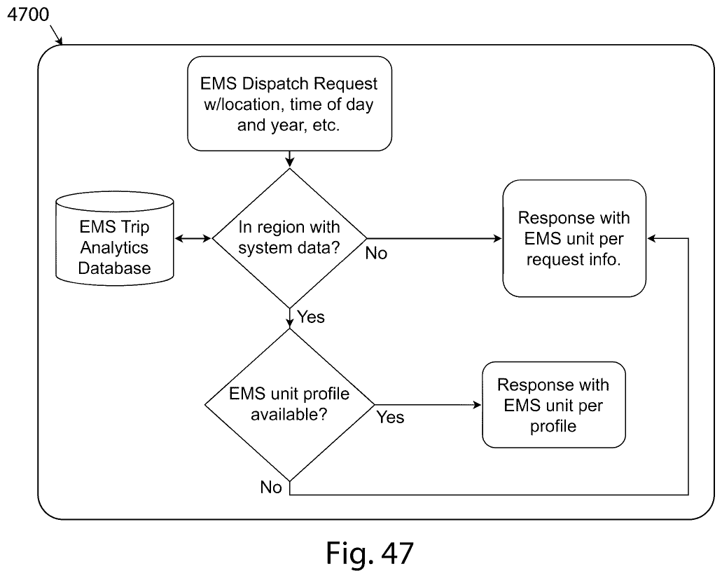

Module 5. Intelligent automated dispatch service, (see FIG. 20) providing ML forecasting for future emergencies and non-emergencies to automate an EMS dispatch service. The method of the system (see reference 4700 FIGS. 47 and 29) allows for intelligent EMS dispatch management in accordance with the EMS unit profile. An EMS dispatch request is sent for a specific location or specific time. The method of the system analyzes the request and compares it with available EMS trip data form the database. The method of the system then indicates if a matching EMS Unit profile is found and is available to respond.

Module 6. Connects the system to third party devices which can automatically determine if a patient falls, is injured or sick, to dispatch a 911 Ambulance. This method of the system uses these sensors as inputs to the ML model for call prediction (see FIG. 10) to become more intelligent. The method of the system is interoperable between all smart systems and enables continuous learning between interoperable ‘smart systems’, such as smart cities (Module 11) and autonomous dispatching (Module 12), providing optimal traffic routes, and preparing traffic for future 911 calls, and EMS transports. Smart engineering systems (Module 13), smart logistics systems (Module 14), and smart grids (Module 15) provide interoperable support and inputs to allow each other to become more intelligent and efficient.

Module 7. Provides intelligent Mass Casualty Incident (MCI) coordination for operations commanders. This method of the system (see reference 6600 on FIG. 66) suggests proper destinations based on real-time data and ML forecasting, factoring bed availability 6605, supplies and staff availability in hospital 6604, and future surges 6603. The method of the system predicts surges in demand, preparing receiving facilities to handle increased patient loads, by having the proper number of staff, resources, and beds available. The method of the system suggests the shortest trajectory 6601 and destination to provide the most, optimal, and efficient care track for the patient to receive definitive care.

Module 8. Is a platform of communication to decrease radio chatter and share real-time information. Receiving emergency department can continuously monitor inbound patients, via mirror imaging diagnostic tools such as EKG, vitals, SPO2, point of care labs, point of care ultrasounds and the like (see reference 6700 on FIG. 67). The module will offer suggestions for bed utilization and placing patients to accommodate the surge of inbound patients, factoring future surges of inbound patients to smooth emergency department operations.

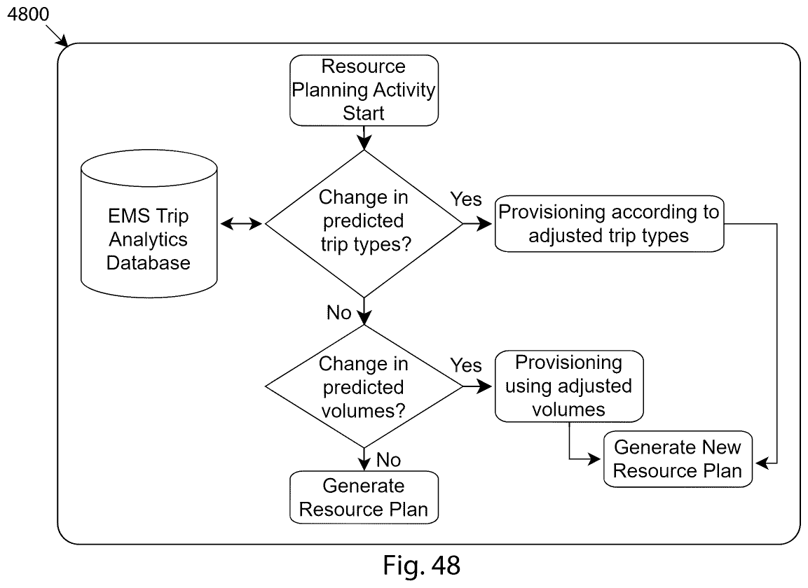

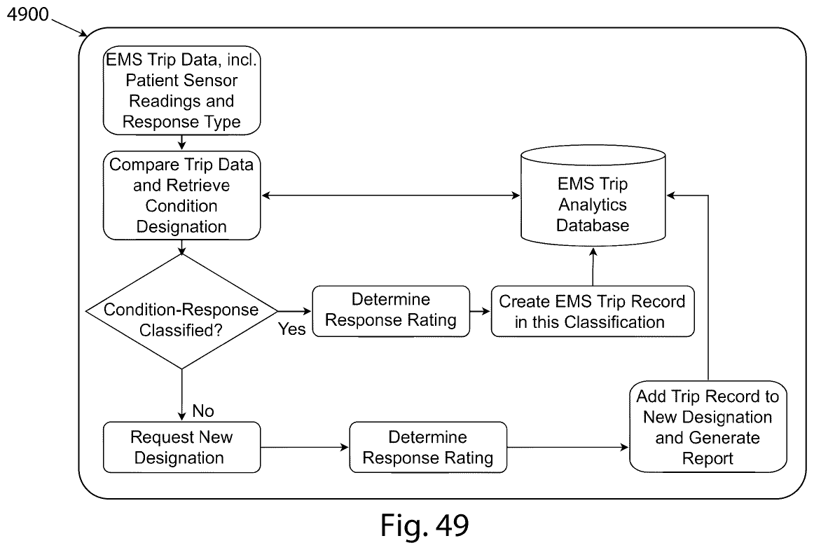

Module 9. Optimizes physician appointment times, inter facility transport times, EMS day-to-day operations, transport routes, and surrounding operations. The output of the trajectory analytics (see reference 6600 FIG. 66) compared to forecasted patient condition profiles 5801 and trajectories 6601, provides the ability to coordinate day-to-day operations. Other inputs include, ML forecasting of patient condition profiles 5601, location of emergency incident, their patient state space and optimal trajectory analyzed by the system, with traffic patterns. Some outputs of the method of the system include reports to optimize, appointment times, pick up and drop off times. The method of the system tracks EMS trip data, including time, pick-up location, and patient diagnosis in order to acquisition resources for the given day. This method of the system (Module 9) (see reference 4800 on FIG. 48) illustrates how patient data analytics aids in the management of resource planning in EMS. The system analyzes patient data form EMS and Trip queries and predicts changes in trip type (or call cases) and trip volumes (call volumes) and adjusts provisioning including material and human resources to generate adequate EMS resource plans. This method allows to increase preparedness and for efficient use of available resources in EMS. This method of the system (Module 9) (see reference 4900 on FIG. 49) illustrates how EMS information is managed and how new trip records and designation in trip cases are added. EMS trip data including response type such as advance life support or basic live support, and patient sensors reading such as EKG and SPO2 readings are added to the system and compared differentiated with other retrieved conditions form the EMS Trip database. The system verifies if a given condition or response has been classified already. In this case the response is rated and added to existing classification. If not, a report is generated, and a new designation is created and added to the EMS trip record database.

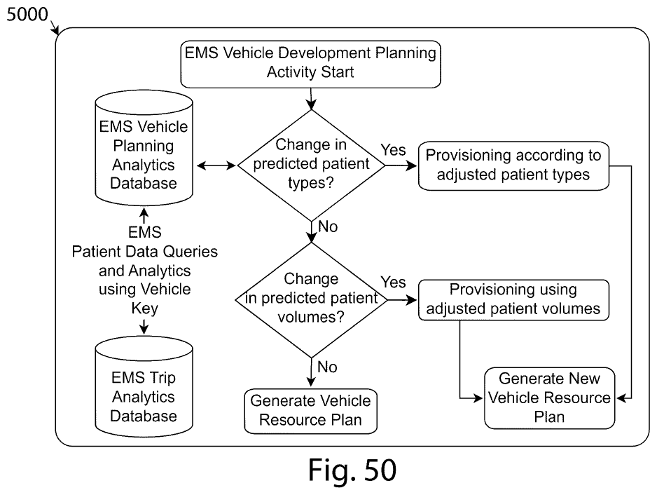

Module 10. Uses ML to predict equipment and personnel needs based on the time of day and location of new EMS trip request. This method of the system uses learning to predict the type and time of day/year trip volumes for acquisition and provisioning of resources, including personnel, units and equipment to meet operational efficiency for the given day, week, month, preference determined by the user. This method of the system (Module 10) (see reference 5000 on FIG. 50) shows how the system aids in the planning of EMS vehicle development. The method of the system assesses changes in expected patient types and volumes by analyzing data form EMS patient and vehicle queries and adjusts provisioning including material and supplies to generate adequate vehicle development plans, to be used by industry manufacturers to determine EMS unit configurations and volumes.

Module 11. Connects to smart city to apply ML forecasting of emergency events, to decrease response time, by aide light changes, routes, update traffic, positive feedback to optimize routes of public transit, inform of delays, etc. The method of the system connects to smart city to use forecasted emergency transports routes and destinations to collaborate efficient, traffic patterns, transport routes for EMS units to get to destination. The method of the system provides bi-directional data to assist in Smart city calculations for energy consumptions, policy making, risk management, financial modeling from EMS and healthcare setting in municipal district.

Module 12. Automatically dispatched emergency response vehicles, and connects to a smart city, and autonomous driving vehicles, to coordinate optimal routes and traffic patterns. This method of the system can automatically pre-position, and dispatch EMS units based on ML forecasted PCP, and location (see reference 5700 on FIG. 57).

Module 13. Connects to smart engineering systems to automate the design method, and manufacturing process to provide volumes and dimensions needed for the environment against forecasted patient cases and loads (see reference 5000 on FIG. 50). This method of the system takes the outputs from the supply provision methods (see FIG. 14 and FIG. 68), to use them as inputs to the smart engineering system to provide the optimal number of supplies needed for projected demand.

Module 14. (see reference 6800 on FIG. 68) Connects with smart logistics systems 6801 to provide optimal coordination of transportation of products, and manufacturing of products. This method of the system takes the outputs from the supply provision methods 6802, to use them as inputs to the smart logistics platform (see FIG. 14 and reference 6801 on FIG. 68). Some outputs may include, coordination of cargo vessels, coordination of transport trucks, industry manufactures preparing for rise in needs of certain materials to produce certain supplies, and the like.

Module 15. Connects to smart grids, to measure carbon emissions, and other energy consumption from the real-world environment, from the start of the manufacturing process to use of product in the real world. This method of the system can provide the most energy efficient route of transport for products, and transportation routes for vehicles. The method of the system can connect to other intelligent carbon emissions models, and provide inputs based on the outputs of the system for supply provisioning methods (see FIG. 14 and reference 6801 on FIG. 68) and EMS transportation methods from call predictions (see FIG. 10).

Module 16. Is a secure platform which provides connectivity between smart system applications in other industries to coordinate together, autonomously recognize and share ML projections to provide more accuracy. This method of the system uses learning to provide correlations and bi-tri-nth-directional flow of analysis to incorporate all real data and forecasted data to provide more accuracy in each industry domain and application. The method of the system provides each smart system with more accurate models by combining and providing a multi-directional flow of information, allowing each systems output to be the other systems input in a real-life environment between all smart systems (see reference 2800 on FIG. 28).

Module 17. Applies natural language processing (NLP) and ML to import data entry into the appropriate documentation platform from speech assessment to text, and visual assessment to text. Data entry automatically goes to proper care team for patient, either specialty care team, ER nurse for triage, and pre-hospital provider to decrease documentation time for providers increasing at patient side time (see reference 6700 FIG. 67).

Module 18. Uses ML to forecast supply provision and acquisition based on predicted surges in demand, call prediction (see FIG. 10, FIG. 11 and FIG. 12) patient cases, patient condition profiles, and resource distribution. The method of the system supplies manufacturers with acquisition forecasting to regulate production and prepare for surges in demand (see reference 6800 on FIG. 68).

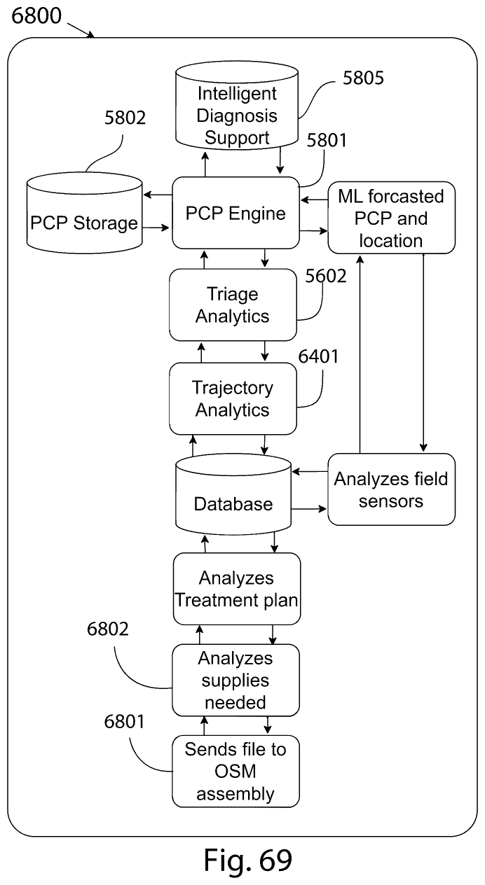

Module 19. Uses ML forecasting to regulate supply production by sending product file for production to On-Site Manufacturing located at facility. This method of the system regulates production to meet demand based on predicted surges, patient cases, patient care profile, and resource distribution (see reference 6900 FIG. 69). The inputs of this method include the outputs from methods in FIG. 14 and FIG. 68. The outputs of the method are the supplies and medical device needed to meet patient demand. The method of the system coordinates the assembly of supplies by regulating the production of the on-site manufacturing assembly line.

Module 20. Coordinates with 3rd party services, warehouses, and other CPS systems to allow industry manufacturers and warehouses to prepare for surges in demand. This method of the system assists in warehouse organization for healthcare sector products, breakdown of supply parts. The method of the system determines where and when supplies will need to be disbursed, and coordinates with each stakeholder in the supply chain process to streamline communication, coordination, and operations to get supply load at destination. The inputs of this method are the outputs of the methods in FIG. 14 and FIG. 68. Some outputs of the method include materials needed to produce supplies, coordination of warehouse storage to fit supplies, and the like.

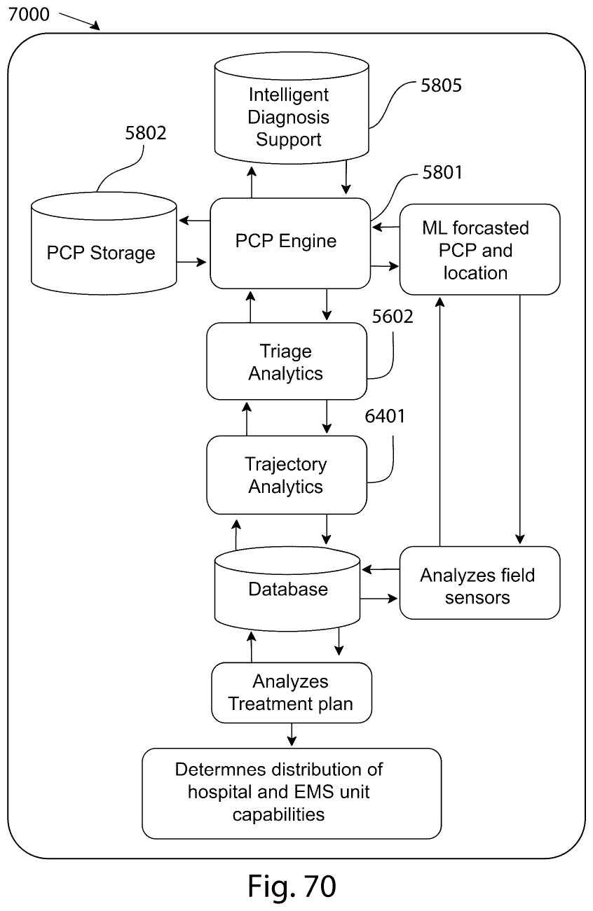

Module 21. Enables industry stakeholders to determine hospital location, department volumes and capabilities and proper EMS unit capabilities to meet localities patient demand. This method of the system (see reference 7000 on FIG. 70) uses ML to forecast future surges in demand and specific patient condition profiles and offers suggestions for optimal distribution of EMS units, hospitals, clinics, bed availability, specialty resource availability, and resources available based on forecasted patient case load. The method of the system uses the inputs of the learned outcomes, some examples include the intelligent dispatch system (see FIGS. 56, and 57), Patient Condition Profile generator (see reference 5800 on FIG. 58), and trajectory analytics (see reference 6600 on FIG. 66). Some outputs include the best location of hospitals, the optimal capacity and capabilities of different hospital units, and the like.

Module 22. Provides industry stakeholders with feedback to determine quality of patient outcome from medical devices, pharmaceutical agents and cost efficiency. Information is sorted and shared with all industry stakeholders, medical device companies, pharmaceutical companies, policy makers, administrators, government officials. This method of the system enables insurance companies, policy makers, and regulatory bodies to oversee patient benefit, and cost benefit to products entering the healthcare field. The method of the system (see reference 7100 on FIG. 71) uses ML forecasting to compare product effectiveness with future surges in demand to provide projections of patient benefit, cost benefit and impact on healthcare field. This provides accountability to pharmaceutical industries and medical device companies on product effectiveness, and real-time monitoring for government officials for return on investment, support for intelligent budget allocation, and industry administrators for more intelligent purchasing.

Module 23. Provides stakeholders such as insurance companies, federal governments, medical device companies, etc. with more accurate models, connecting their financial software with real time feedback, and learning from forecasted events from the customers real world environment from the outputs of the method (see reference 7100 on FIG. 71). The method of the system uses learning to provide bi-directional flow of analysis for more accurate risk management models based on forecasted patient volume, cases, associated supplies and usage around patient trajectory.

Module 24. Provides real-time and ML forecasted financial calculations based on ML forecasting outputs previously described, related to patient condition profiles, trajectory, cost of care and treatment (see reference 7100 on FIG. 71) and the like. The end user are the industry stakeholders, such as policy makers, regulatory bodies, insurance companies and the like. This method of the system provides discrepancies in cost, contradictions in policies, and current real-world ROI and projected ROI to real world conditions. The method of the system provides suggestions to make improvements, simplify processes, etc. insurance adjustments to be made, against forecasted health events in an environment, age group, etc. The method of the system provides suggestions to make improvements, simplify processes, etc.

Module 25. Automatically process insurance claims for billing companies and MACs (Medicare Administrative Contractor). This method of the system uses learning to pull necessary requirements to file insurance claims and automatically submits them.

Module 26. Identifies redundancies in policies, multiple and fraudulent insurance claims, and other infractions for example, the ratio between reimbursement rate:cost of care, multiple claims, and fraudulent claims.

Module 27. Utilizes natural language processing (NLP) to maintain up to date with regulatory bodies, policies, in order to automate documentation for providers to be compliant with insurance codes, and regulations. This method of the system automatically submits insurance claims to private and public insurances. The method of the system ensures managers and administrators to continuously remain compliant.

Module 28. Generates automated reports, analytical studies, market surveillance surveys, epidemiological studies (see FIG. 15) and reporting, with real-time and ML forecasted data, ‘weather map’ or ‘heat map’ style (see reference 4301 on FIG. 44) or other data visualization technique for user preference (see FIG. 1 and FIG. 2).

It is important to note that while the present embodiments has been described in the context of a fully functioning data processing system, those of ordinary skill in the art will appreciate that the processes of the present embodiments are capable of being distributed in the form of a computer readable medium of instructions and a variety of forms and that the present embodiments applies equally regardless of the particular type of signal bearing media actually used to carry out the distribution. Examples of computer readable media include recordable-type media, such as a floppy disk, a hard disk drive, a RAM, CD-ROMs, DVD-ROMs, and transmission-type media, such as digital and analog communications links, wired or wireless communications links using transmission forms, such as, for example, radio frequency and light wave transmissions. The computer readable media may take the form of coded formats that are decoded for actual use in a particular data processing system.

While the embodiments have been described in conjunction with specific embodiments, it is evident that many alternatives, modifications, and variations will be apparent to those skilled in the art in light of the foregoing description. Accordingly, the present embodiments attempt to embrace all such alternatives, modifications, and variations that fall within the spirit and scope of the appended claims. Accordingly, the specification and drawings are to be regarded in an illustrative rather than a restrictive sense. Throughout this specification and the drawings and Figures associated with this specification, numerical labels of previously shown or discussed features may be reused in another drawing Figure to indicate similar features. It is also understood that the phraseology or terminology employed herein is for the purpose of description and not of limitation. It is to be understood that the description above contains many specifications, these should not be construed as limiting the scope of the embodiments but as merely providing illustrations of some of the personally preferred embodiments.

### REFERENCES—WHICH ARE INCORPORATED HEREIN BY REFERENCE

1. Winter, V., Schreyögg, J. & Thiel, A. Hospital staff shortages: Environmental and organizational determinants and implications for patient satisfaction. Health Policy (New. York). 124, 380-388(2020).
2. Medical, E. & Council, S. EMS WORKFORCE SHORTAGE IN NYS: (2019).
3. Long, M., Care, T., Payer, S. & Commission, H. Network United New York.
4. Usher, A. D. Medical oxygen crisis: a belated COVID-19 response. Lancet (London, England) 397, 868-869 (2021).
5. Fischer, R. et al. Comparison of portable oxygen concentrators in a simulated airplane environment. Respir. Med. 107, 147-149 (2013).
6. Sacco, E. & Moon, S. K. Additive manufacturing for space: status and promises. Int. J. Adv. Manuf. Technol. 105, 4123-4146 (2019).
7. Zhou Y, Chen C, Zhu S, et al. A printed, recyclable, ultra-strong, and ultra-tough graphite structural material. Materials Today. 2019; 30:17-25. doi:10.1016/J.MATTOD.2019.03.016
8. Magnetic Liquid Metal (Fe-EGaIn) Based Multifunctional Electronics for Remote Self-Healing Materials, Degradable Electronics, and Thermal Transfer Printing _ Enh.
9. Sustainable production of highly conductive multilayer graphene ink for wireless connectivity and IoT applications _ Enhanced Reader.
10. Flandin L, Bidan G, Brechet Y, Cavaille J Y. New Nanocomposite Materials Made of an Insulating Matrix and Conducting Fillers: Processing and Properties.
11. Trivedi, M. et al. Additive manufacturing of pharmaceuticals for precision medicine applications: A review of the promises and perils in implementation. Addit. Manuf. 23, 319-328 (2018).

We claim:

1. A method to optimize human resource management within emergency medical services (EMS), comprising the steps of: receiving inputs of one or more of the group selected from the list of traffic conditions, weather, incident location of emergency or non emergency call, call type, dispatch type, latitude and longitude of incident location, age, sex, chief complaint, incident date and time, holiday, day of the week, call classification, emergency department population status, incoming EMS service requests, available medical consumables, available medical non consumables, available staff, Cellular triangulation of staff, Cellular triangulation of ambulance or other mobile EMS equipment, Cellular triangulation of service base sites, GPS location of staff, GPS location of the service base sites, GPS location of ambulance or other mobile EMS equipment, identified location of needed services, dispatch requests, time of dispatch requests, latitude and longitude of dispatch requests, hospital census counts, duration of patient admittance in hospital, admitting diagnosis in hospital, discharge diagnosis in hospital, unit transition patterns of patients, unit transition date and time, unit admitting date and time, admitting unit in hospital, on-site manufacturing capabilities,
providing a scheduling module for building one or more predictive assessment values; the scheduling module outputting automatically a suggested scheduling template which matches EMS call volume appropriately to provide the maximum ambulance per call ratio per shift and is configured to ensure maximum revenue for an EMS agency; and employing one or more machine learning models to generate the one or more predictive assessment values that relate to the comparison of the inputs of the evolution of data over a time interval using machine learning; employing a global positioning systems (GPS) device to provide geo-location information of EMS equipment and personal communication devices in order to assess availability; and displaying to a user the results of the comparison, including the one or more comparison outcomes, and one or more predicted assessment values that relate to the comparison of inputs or to the predicted evolution of data over a time interval using machine learning.

2. The method of claim 1, wherein scheduling module uses incident time, location, and type to predict and forecast future call volume type and location in real-time.

3. The method of claim 1, wherein the scheduling model outputs one or more of automatic scheduling, tracking of epidemiological data for research, and resource/supply management.

4. The method of claim 1, wherein the scheduling model outputs predicted medical consumable needs by agency.

5. The method of claim 1, wherein the machine learning models utilize Ensemble learning+neural networks, Decision tree and deep reinforcement learning via call simulations to create Model-free algorithms.

6. The method of claim 1, wherein the system machine learning models learns and analyzes a patient condition profile to determine a patient trajectory based on patient outcome and time to treatment.

7. The method of claim 6, wherein the system analyzes a receiving entities profile for characteristics including at least one of bed availability, staff availability, supply availability and predicted patient load by patient condition for that facility; wherein the profiles are encoded to enable storage and processing using a digital computing device; wherein the system analyzes the encoded profiles to determine optimal outcome and coordinates EMS unit response and patient trajectory based on current and forecasted conditions.

8. The method of claim 1, wherein the system has components for a Patient Condition Profile engine and a Patient Condition Profile Database, which receives information gathered from an emergency dispatch caller and generates, stores and updates the Patient Condition Profile in real time; wherein data of the Patient Condition Profile is derived from a caller's answers to a 911 call taker's questions according to predetermined scripts; wherein the Patient Condition Profile output assists the system in determining a response based on predetermined characteristics, including optimal outcome.

9. The method of claim 1, wherein the system comprises an EMS Response Module that continuously analyzes and updates a Patient Condition Profile to provide intelligent EMS response to the patient's condition; wherein the system adapts to input of a changing patient condition; wherein an optimal choice of EMS unit response is determined by analyzing the patient, EMS unit, and receiving entity profiles for the most optimal patient outcome.

10. The method of claim 1, wherein the system comprises an EMS Unit Provisioning Module that aligns healthcare resources, including supply and personnel distribution;
wherein the system aligns acquisition and provisioning to ensure the supplies needed to respond to the patient condition are present on the responding EMS unit; and
wherein supplies may comprise at least one of medications, equipment, and consumable and non-consumable supplies.

11. The method of claim 1, wherein the system comprises coordinated and Intelligent Predictive Analytics with visual outputs from analytics of a Visualization Function Map which includes the step of displaying forecasting of events for patient demand and supports quantitative reasoning;
wherein the events can be filtered by at least one of date, time, type of call, disease, and diagnosis.

12. The method of claim 11 wherein the system comprises the step of simulating emergency responses to support decision making at all levels, including municipalities.

13. A system to optimize human resource management within emergency medical services comprising: a plurality of modules which provide functions to distribute data which is obtained from the plurality of modules to each stakeholder in real-time, to redistribute data which is obtained from the plurality of modules in real-time, and to retrain its own ML models in real-time; wherein each module provides a specific application for a targeted solution, provides the system a source of sensors for data gathering for the system to train itself in real-time within the specific healthcare systems environment in which it is implemented and redistribute its data accordingly, either to the stakeholder, back to the system itself, or another module.
